(19) United States
(12) Patent Application Publication
(10) Pub. No.: US 2017/0119874 A1
(43) Pub. Date: May 4, 2017

(54) HUMAN CYTOMEGALOVIRUS VACCINE COMPOSITIONS AND METHOD OF PRODUCING THE SAME

(71) Applicants: INSTITUTE FOR RESEARCH IN BIOMEDICINE, Bellinzona (CH); FONDAZIONE CARIPLO, Milano (IT)

(72) Inventors: Antonio Lanzavecchia, Porza (CH); Laurent Perez, Minusio (CH)

(21) Appl. No.: 15/305,494

(22) PCT Filed: Apr. 23, 2015
(86) PCT No.: PCT/EP2015/000843

§ 371 Date: Oct. 20, 2016

(30) Foreign Application Priority Data
Apr. 23, 2014 (EP) PCT/EP2014/001086
Apr. 30, 2014 (EP) PCT/EP2014/001168

(51) Int. Cl. A61K 39/245; C07K 14/005

(52) U.S. Cl. CPC A61K 39/245; C07K 14/005; C12N 2710/16122; C12N 2710/16134; C12N 2710/16151; A61K 2039/645

(57) ABSTRACT

The present invention provides for a vector and a gene expression system for producing a soluble pentameric protein complex comprising the HCMV glycoproteins UL128, UL130, UL131, gH and gL or sequence variants thereof, as well as vaccine compositions comprising the same. The present invention further provides for a vaccine composition for use in prophylactically or therapeutically vaccinating against HCMV infections. Also disclosed are methods of producing the inventive vaccine. Furthermore, the present invention pertains to methods of vaccination of humans with the inventive vaccine composition.

### FIELD OF THE INVENTION

The present invention relates to the field of HCMV vaccination, in particular to vaccine compositions for use in the vaccination against human cytomegalovirus, methods of producing the same as well as to methods of vaccination.

### BACKGROUND OF THE INVENTION

Human Cytomegalovirus (HCMV) is a ubiquitously distributed β-herpesvirus member of the family of the Herpesviridae family. The virus spreads via excretion in nearly all body fluids, such as urine, saliva, vaginal secretions, semen or breast milk. Especially infants and toddlers shed high amounts of virus for months or even years and represent a substantial risk for transmitting the virus to pregnant women by saliva or urine. Sexual transmission of the virus is a common way of infection in adults.

HCMV represents a major threat for the developing fetus and immunocompromised patients. For the latter group, in particular solid organ transplants (SOT) or hematopoietic stem cell transplant (HSCT) recipients are at great risk of HCMV infection. Despite active monitoring in these patients and management with antiviral drugs, the incidence of HCMV infection is high, ranging from 20% to 70% in the first year post transplantation (Kotton, Conn., Nat Rev Nephrol 2010; 6:711-721; Beam et al., Curr Infect Dis Rep 2012; 14:633-41; Ariza-Heredia et al., Cancer Lett 2014; 342:1-8). Infections can occur as newly acquired infection, which is frequently observed in HCMV seronegative recipients receiving SOT from seropositive donors, or as re-current infection due to reactivation of latent virus in seropositive recipients.

For the developing fetus HCMV is the most common cause of in utero viral infections in North America and Europe, affecting 0.5-2% of newborns annually. Congenital HCMV infection can lead to symptomatic diseases at birth and also cause developmental disabilities in children. Approximately 10% of congenitally infected infants have signs and symptoms of disease at birth, and these symptomatic infants have a high risk for demonstration of subsequent neurologic sequelae. CMV infection and CMV-induced damage in the fetus may also cause spontaneous abortion or prematurity.

Typically, cases of congenital HCMV syndrome present with an involvement of multiple organs including splenomegaly, hepatomegaly, prolonged neonatal jaundice, pneumonitis, thrombocytopenia, growth retardation, microcephaly and cerebral calcifications. Organ damage is thought to be caused by HCMV replication in target organs like the central nervous system of the foetus and indirectly by HCMV-induced placental dysfunction. Permanent impairments mostly affect the central nervous system and include progressive hearing loss, spastic tetraplegia, mental retardation and visual impairments (Watemberg et al. Clin Pediatr (Phila). 2002; 41(7):519-22). Nearly 14% of children with congenital HCMV infection suffer from sensorineural hearing loss (SNHL), and 3-5% of children with congenital CMV infection suffer from bilateral moderate to profound SNHL. About 15-20% of children with moderate to profound permanent bilateral hearing loss were associated with HCMV infection (cf. Grosse et al.) Clin Virol. 2008; 41(2):57-62).

Maternal seropositivity prior to conception protects against congenital transmission, and both maternal humoral and cellular immunity are likely to contribute to the protection. Antibodies in particular are important for preventing congenital infection, serving as the first line of defense against maternal infection. According to the results of a small, non-randomized study in pregnant women with primary HCMV infection it may also play a role in preventing transmission to the fetus, in which a passive immunity of monthly infusions of HCMV hyperimmune human IgG (HCMVHIG) (200 mg/kg maternal weight) was effective in about 60% of the cases in protecting against congenital HCMV infection, suggesting that developing a HCMV vaccine may be feasible for preventing congenital HCMV infection and its sequelae.

However, in a more recent phase II, randomized, placebo-controlled, double-blind study on 123 pregnant women, the rate of congenital infection was 30% in the hyperimmune human IgG group and 44% in the placebo group, with no significant difference between the two groups. The finding that hyperimmune globulin did not significantly modify the course of primary CMV infection may be due to the low amounts of neutralizing antibodies in the IgG preparation and suggests that high amounts of antibodies may be required to block virus spread.

Wild-type HCMV as a prototype-member of the β-herpesvirus family possess a double-stranded DNA (dsDNA) genome of around 235 kb, which is longer than all other human herpesviruses and one of the longest genomes of all human viruses in general. It is estimated that the HCMV genome codes for more than 165 open reading frames (ORFs). The mature virions are about 200 nm in diameter and are comprised of more than 50 viral proteins, including viral capsid proteins, tegument proteins and envelope glycoproteins.

The HCMV genome has the characteristic herpesvirus class E genome architecture, consisting of two unique regions (unique long UL and unique short US), both flanked by a pair of inverted repeats (terminal/internal repeat long TRL/IRL and internal/terminal repeat short IRS/MS). Both sets of repeats share a region of a few hundred baise pairs (bps), the so-called “a” sequence; the other regions of the repeats are sometimes referred to as “b” sequence and “c” sequence. The genome exists as an equimolar mixture of four genomic isomers by inversion of UL and US regions (Murphy et al. Curr. Top. Microbiol. Immunol. 2008, 325, 1-19). The first complete HCMV genome of the CMV strain AD169 was published in 1990 and was the largest contiguous sequence generated by M13 shotgun cloning and Sanger sequencing at the time. Of the more than 165 genes encoded by HCMV, less than one-fourth are essential for viral replication and are conserved across herpesvirus families. The gene products ORFs 37-60 are e.g. detected following in vitro infection of CD34+ primary hematopoietic progenitor cells (HPCs) or myeloid lineage cells and cell line models.

Although clinical isolates of HCMV replicate in a variety of cell lines, laboratory strains, such as e.g. AD169 or Towne, replicate almost exclusively in fibroblasts. This restriction in viral tropism results from the reiterated, serial passage of the virus in fibroblasts and is a marker for viral attenuation. Mutation, which cause the loss of epithelial cell, endothelial cell, polymorphonuclear leukocyte and dendritic cell tropism have been mapped to three ORFs of HCMV, namely UL128, UL130 and UL131. Mutations in any one of these ORFs in the “FIX” clinical isolate of HCMV blocked endothelial cell tropism. Subsequent experiments have shown that the repair of a single nucleotide insertion in the UL131 ORF restored the ability of the AD169 HCMV strain to infect endothelial as well as epithelial cells.

Some viral glycoproteins such as gM, gN and gB are used by HCMV to infect different cell types, while glycoprotein complexes containing gH and gL mediate cell type-specific virus entry. A pentameric complex comprising gH, gL, pUL128, pUL130 and pUL131 (also referred to as gHgLpUL128L) was shown to be required for infection of endothelial, epithelial and myeloid cells by clinical HCMV isolates. In vitro cultured viruses with mutations in the UL128-131 locus have lost tropism for endothelial and epithelial cells, but have retained the expression of the gHgL dimer, which is sufficient to infect fibroblasts.

Because of the high incidence rate of HCMV infections and its impact on public health, considerable efforts have been made in the last decade to develop vaccines capable of preventing HCMV infection. Two general approaches have been taken for vaccine design: One strategy in vaccine design utilizes modified virus vaccines (MMVs), the second one employed individual antigen vaccines (IAVs).

A typical strategy chosen for MMVs is to modify HCMV in a way that the virus would be attenuated or replication-defective with the advantage of presenting all relevant antigens to the immune system that correspond to wild-type HCMV during infection. MMV approaches taken include live attenuated Towne and AD169 viruses, Towne/Toledo chimeric viruses and dense body (DB) vaccines (cf. Fu, T M et al., Vaccine 2014, May 7; 32(22):2525-2533).

The use of live, attenuated HCMV vaccines induce both, antibody responses as well as broad-based cellular responses, including cytotoxic CD+ T-cell responses (Heinemann et al., The J. of infectious disease (2006), 193(10): 1350-1360). However, safety considerations regarding the long term risks of a HCMV live-virus approach, including atherosclerosis, immune senescence, reactivation from latency and potentially even Alzheimer's disease have rendered this approach unattractive for the development of a HCMV vaccine (Schleiss, Future Virol. 2013, 8(12):1161-1182).

The IAV approach is designed to present defined one or more viral antigens, which may be delivered in form of recombinant protein, a DNA vaccine or viral vector. Antigens which are typically considered for the IAV approach comprise antigens that are recognized by the dominant humoral or T-cell response, or both, in naturally infected humans. For example, attempts have been made in developing a subunit vaccine based on glycoprotein B (gB), which is an abundant surface glycoprotein of HCMV involved in virus fusion and a target of neutralizing antibodies (nAbs): gB has been shown to elicit T cell and antibody response and it represents the basis of most vaccines developed so far. In recent phase II trials, a MF59-adjuvanted gB vaccine showed modest efficacy in preventing infection of seronegative women and only reduced duration of viremia in transplant recipients. The gB vaccine used was produced recombinantly, differing from the natural gB glycoprotein, which is membrane-anchored and composed of two subunits linked by disulfide bonds, in that the recombinant molecule was designed as a single molecule with its furin cleavage site mutated and its transmembrane domain deleted. Thus, the soluble gB vaccine is not designed to assemble into a trimeric complex as has been described for the gB of herpes simplex virus-1 (Heldwein et al., Science 2006; 313:217-20). It is therefore unlikely that the recombinant gB vaccine presents with the antigenic structure of that of the wild-type gB glycoprotein in the viral envelope. This altered antigenic structure may explain the finding that most of the antibodies induced by the vaccines lacked virus neutralizing activity, while those neutralizing did not block efficiently infection of epithelial cells. Based on this observation, IAV vaccines have raised the question whether vaccine-induced antibody responses raised against a single viral glycoprotein would be sufficient to induce an antibody response resembling that of natural HCMV infection, in particular with regard to the number of neutralizing antibodies.

Thus, from vaccine design perspectives, regardless of MVV or IAV approaches, the immunological goal is to identify the best target of neutralizing antibodies in natural HCMV infection or a crucial component of such immunity in order to produce a vaccine that induce a neutralizing antibody response equal or even better than that induced by HCMV infection. However, limitations are imposed by the extent of how accurately or faithfully human immune responses can be characterized by in vitro or animal models: Many variables in the immune assays including HCMV strains used can lead to contradictory or even misleading results, such as the recent findings of epithelial tropism-deficiency of the AD169 HCMV laboratory strains, which have been widely used for vaccine development. In the AD169 HCM strain mutations as the result of fibroblast adaptation have accumulated which result in a deficiency of the AD129 strain to produce the pentameric gH protein complex due to a frame-shift mutation in the UL128-131 locus (Wang et al., Proc Natl Acad Sci USA 2005; 102:18153-8).

Also, given the striking species-specificity of CMVs, the precise molecular/cellular basis of which is unknown, preclinical studies of HCMV vaccination are generally not feasible in animal models of HCMV infection. HCMV-specific immunogens, including recombinant proteins, virions, dense bodies have all been evaluated for immunogenicity in a number of animals, including mice, rabbits, hamsters, guinea pigs and rhesus macaques, however, these studies do not allow to evaluate efficacy of the different immunogens as vaccines, as HCMV will not replicate in these model organism or cause disease.

It is thus an objective of the present invention to provide a HCMV vaccine composition, which is capable of eliciting an immune response resulting in the formation of a repertoire of neutralizing antibodies that are protective against infection of all cellular targets while minimizing production of non-neutralizing antibodies, i.e. capable of inducing an antibody response of high “specific activity”.

### SUMMARY OF THE INVENTION

The present inventors have identified that vaccine compositions, which comprise the pentameric glycoprotein complex of the HCMV proteins gH, gL, UL128, UL130 and UL131 (also referred to herein as “HCMV pentamer”) as immunogenic components (or subunits), result in the formation of a high number of neutralizing antibodies against HCMV and thus may provide an efficient vaccine against HCMV infection.

More specifically, the inventors have surprisingly found that a vector comprising nucleotide sequences encoding each of the five subunits of this HCMV pentameric glycoprotein complex, i.e. gH, gL, UL128, UL130 and UL131 (also referred to as “immunogenic components” in the following), enables the preparation of a vaccine, which elicits the formation of high numbers of predominantly neutralizing antibodies against HCMV infection of fibroblasts, epithelial, endothelial and myeloid cells. As an underlying mechanism it is assumed that a vector comprising nucleotide sequences encoding each of the five subunits of the HCMV pentameric glycoprotein complex, i.e. gH, gL, UL128, UL130 and UL131, enables a predominantly equimolar expression of these subunits, in particular predominantly 1:1:1:1:1 stoichiometry, thereby resulting in a properly folded HCMV pentameric glycoprotein complex, i.e. a HCMV pentameric glycoprotein complex with the proper protein structure, whereas the formation of single subunits, other subunit assemblies, and/or protein complexes which are not properly folded, which would all result in a less specific antibody response, is largely avoided. In addition, the present invention enables high product yields, since equimolar expression of the subunit is ensured in stably transfected cells. Stable transfection is based on integration into the host genome, whereby the one or more open reading frames comprised by a single vector are typically integrated into the same genomic site having the same transcriptional activity. Accordingly, the nucleotide sequences encoding the five subunits comprised by a single vector according to the present invention are typically integrated into the same genomic site upon stable transfection resulting in a balanced expression, in particular equimolar expression. In contrast, if more than one vector is used, different open reading frames located on the different vectors are typically integrated into different genomic sites. However, in different genomic sites the level of chromatin accessibility for transcription may be different, typically resulting in expression differences of the different ORFs derived from the different vectors. Moreover, differences in the numbers of copies of the vector, which are integrated into the host genome, may occur. In case of the vector according to the present invention, such differences in the vector copy numbers do not impair the balanced expression of the five subunits, since every vector copy comprises a nucleotide sequence encoding gH, a nucleotide sequence encoding gL, a nucleotide sequence encoding UL128, a nucleotide sequence encoding UL130, and a nucleotide sequence encoding UL131. However, if the five subunits are encoded by more than one vector, genome integration of different copy numbers of the different vectors encoding the five subunits typically results in additional expression differences. Thus, a vaccine according to the present invention, which is obtainable by the inventive vector, shows a higher specific activity compared to conventional vaccines against HCMV infection.

In a first aspect, the present invention thus provides for a vector which comprises a transcription system, comprising one or more promoter(s), preferably one or two promoter(s) (which are typically operable in the mammalian cell), which is/are operably linked to one or more open reading frames coding for the above mentioned immunogenic components gH, gL, UL128, UL130 and UL131. Thus, according to the invention in general a single vector encodes all five immunogenic components gH, gL, UL128, UL130 and UL131, preferably each of them in a single copy. In particular, the transcription system of the inventive vector comprises the five immunogenic components gH, gL, UL128, UL130 and UL131 arranged in one or more open reading frames (ORF) whereby usually a promoter is operably linked to each of the at least one open reading frames.

Since the inventive vector is usually used for the preparation of a vaccine for use in mammals, in particular in humans, the vector is in general designed for this use. To this end the vector is preferably suitable for expressing HCMV glycoproteins in a mammalian cell and used in this context, since vaccine preparations are advantageously based on a mammalian expression system for safety aspects including e.g. the provision of an appropriate glycosylation pattern.

Moreover, according to the invention it is preferred that the HCMV pentameric glycoprotein complex, which is obtainable by the inventive vector, is secreted, i.e. released from the cells expressing it into the supernatant. This significantly simplifies the preparation of the protein complex and in particular of the vaccine and is thus very useful in particular for large scale production. To this end the transmembrane domain of the gH subunit is preferably mutated, in particular deleted, e.g. SEQ ID NOs: 21 and 35 or sequence variants thereof. Thus, throughout this description it is understood that a “sequence encoding gH” (or an amino acid sequence for gH) relates preferably to such gH sequences, wherein the transmembrane domain is mutated, preferably deleted.

In a preferred embodiment, the at least one promoter of the inventive vector of the inventive gene expression system may be chosen from any appropriate promoter, in particular any viral promoter and, further, any promoter of herpes virus origin. If more than one promoter is present in the inventive vector, the further promoter of the inventive vector of the inventive gene expression system may be the same as or different from the first promoter. More preferably, the first promoter may be selected from the group consisting of a MCMV, a HCMV, a SV40, a HSV-TK, an EF1-1a and PGK promoter. Accordingly, any further promoter may be selected from the group consisting of a MCMV, a HCMV, a SV40, a HSV-TK, an EF1-1α and PGK promoter as well. Preferably, the first and/or any further promoter is a hCMV major immediate-early promoter (hCMV-MIE promoter), which is also known as hCMV major immediate-early enhancer (hCMV-MIE enhancer). It is also preferred that the first and/or any further promoter is a MCMV promoter (murine CMV promoter).

Accordingly, the inventive vector of the inventive gene expression system comprises by its transcription system nucleotide sequences coding for all above mentioned immunogenic components, preferably as defined by SEQ ID Nos: 3 (UL128), 7 (UL130), 11 (UL131), 21 (gH) and 25 (gL) or sequence variants thereof, which are arranged in at least one open reading frame and whereby a promoter is operably linked to preferably each open reading frame.

More specifically, the at least one open reading frame comprises at least one nucleotide sequence selected from the group consisting of nucleotide sequences encoding an amino acid sequence for gH, gL, UL128, UL130, and UL131, in particular according to SEQ ID NO:21, SEQ ID NO:25, SEQ ID NO:3, SEQ ID NO:7 and SEQ ID NO:11 or sequence variants thereof, whereby the vector comprises each of the nucleotide sequences selected from the group consisting of nucleotide sequences encoding an amino acid sequence for gH, gL, UL128, UL130, and UL131, in particular according to SEQ ID NO:21, SEQ ID NO:25, SEQ ID NO:3, SEQ ID NO:7 and SEQ ID NO:11 in at least one open reading frame linked to at least one promoter.

Accordingly, the nucleotide sequences encoding gH, gL, UL128, UL130 and UL131 are preferably the nucleotide sequences encoding the amino acid sequences according to SEQ ID NO:21, SEQ ID NO:25, SEQ ID NO:3, SEQ ID NO:7 and SEQ ID NO:11 or sequence variants thereof. Even more preferably the nucleotide sequences encoding the amino acid sequences according to SEQ ID NO:21, SEQ ID NO:25, SEQ ID NO:3, SEQ ID NO:7 and SEQ ID NO:11 are the nucleotide sequences according to SEQ ID NO:22, SEQ ID NO:26, SEQ ID NO:4, SEQ ID NO:8 and SEQ ID NO:12 or sequence variants thereof.

Examples for sequence variants of gL and gH are e.g. SEQ ID NO:35, SEQ ID NO:37, while sequence variants of pUL130, pUL131 are e.g. SEQ ID NO:31 and SEQ ID NO:33. Any order for an arrangement of the nucleotide sequences coding for the above defined immunogenic components may be chosen as long as nucleotide sequences encoding each of the immunogenic components gH, gL, UL128, UL130 and UL131 are contained, preferably as a single copy, in a single vector. Preferably, the arrangement is such that the nucleotide sequences coding for gH and gL are located adjacent to each other and/or the nucleotide sequences coding for UL128, UL130, and UL131 are located adjacent to each other. More preferably, the arrangement of the nucleotide sequences coding for UL128, UL130 and UL131 within the open reading frame is chosen such that they are located in the above order in 5′-3′ direction.

According to one embodiment, the inventive vector comprises by its transcription system one single open reading frame comprising nucleotide sequences which code for all of the immunogenic components gH, gL, UL128, UL130 and UL131, preferably each in a single copy. Preferably the nucleotide sequences encode amino acid sequences according to SEQ ID No: 3, 7, 11, 21 and 25 or sequence variants thereof. Preferably, one promoter is operably linked to this one open reading frame.

By another embodiment, the inventive vector comprises by its transcription system more than one promoter operably linked to more than one open reading frame comprising nucleotide sequences which code for the immunogenic components gH, gL, UL128, UL130 and UL131, preferably according to SEQ ID No: 3, 7, 11, 21 and 25 or sequence variants thereof. The immunogenic components encoded by the underlying nucleotide sequences, e.g. SEQ ID NOs: 4 (UL128), 8 (UL130), 12 (UL131), 22 (gH), and 26 (gL) or other nucleotide sequences coding for gH, gL, UL128, UL130 and UL131 (whereby such other nucleotide sequences also encoding SEQ ID NO:21, SEQ ID NO:25, SEQ ID NO:3, SEQ ID NO:7 and SEQ ID NO:11 due to the degeneracy of the genetic code are preferred), may be allocated in any possible arrangement (and any order) to 2 to 5 open reading frames (each open reading frames operatively linked to a promoter), i.e. (a1) two open reading frames comprising two and three of the nucleotide sequences coding for the above immunogenic components, respectively, or (a2) two open reading frames comprising one and four of the nucleotide sequences coding for the above immunogenic components, respectively (b) three open reading frames (two of which comprise two of the above nucleotide sequences, while a third open reading frame comprises the remaining nucleotide sequence such that all five immunogenic components are encoded by the inventive vector. Less preferred are vectors comprising four or five open reading frames for encoding all of the above five immunogenic components.

Thus, the vector according to the present invention preferably comprises no more than two promoters operably linked to at least one open reading frame comprising at least one nucleotide sequence selected from the group consisting of a nucleotide sequence encoding gH, a nucleotide sequence encoding gL, a nucleotide sequence encoding UL128, a nucleotide sequence encoding UL130 and a nucleotide sequence encoding UL131; or sequence variants thereof. In other words, it is preferred that the vector according to the present invention comprises either (i) one single promoter operably linked to one single open reading frame comprising a nucleotide sequence encoding gH, a nucleotide sequence encoding gL, a nucleotide sequence encoding UL128, a nucleotide sequence encoding UL130, and a nucleotide sequence encoding UL131, or sequence variants thereof; or (ii) exactly two promoters, each of them operably linked to one open reading frame, whereby the first open reading frame comprises 1-4 nucleotide sequence(s) selected from the group consisting of a nucleotide sequence encoding gH, a nucleotide sequence encoding gL, a nucleotide sequence encoding UL128, a nucleotide sequence encoding UL130 and a nucleotide sequence encoding UL131, or sequence variants thereof and the second open reading frame comprises the nucleotide sequences encoding those of gH, gL, UL128, UL130 and UL131 or sequence variants thereof, which are not comprised by the first open reading frame.

It is understood that by an open reading frame comprising more than one, e.g. two, three, four, or five, of the nucleotide sequences encoding gH, gL, UL128, UL130 and UL131 or sequence variants thereof, it is meant herein that each of said more than one, e.g. two, three, four, or five, of the nucleotide sequences encodes a different immunogenic component. Thus, an open reading frame comprising more than one, e.g. two, three, four, or five, of the nucleotide sequences encoding gH, gL, UL128, UL130 and UL131 or sequence variants thereof, as used herein does not refer to an open reading frame comprising multiple copies of the same nucleotide sequence or multiple nucleotide sequences each encoding the same immunogenic component. More preferably, two open reading frames are provided by the inventive vector of the inventive gene expression system, one of them comprising the nucleotide sequences encoding two of the above five immunogenic components according to SEQ ID NOs 3, 7, 11, 21, and 25, while the other open reading frame encodes for the other three immunogenic components. If two open reading frames are provided by the transcription system of the inventive vector of the inventive gene expression system, the transcription system particularly preferably comprises (a)(i) a first promoter operable in a mammalian cell operably linked to (a)(ii) a first open reading frame (ORF), which comprises a nucleotide sequence, which preferably encodes gH and gL, more preferably the nucleotide sequence encodes SEQ ID NO:21 and SEQ ID NO:25, or sequence variants thereof, and (b)(i) a second promoter operable in said mammalian cell and operably linked to b(ii) a second open reading frame (ORF), which comprises a nucleotide sequence preferably encoding UL128, UL130, and UL131, more preferably the nucleotide sequence encodes SEQ ID NO:3, SEQ ID NO:7 and SEQ ID NO:11 or sequence variants thereof. The present inventors have surprisingly found that such a configuration of the vector enables an equimolar expression of the subunits gH, gL, UL128, UL130 and UL131 of the HCMV pentameric glycoprotein complex, i.e. a 1:1:1:1:1 stoichiometry of the subunits gH, gL, UL128, UL130 and UL131. This is not only superior to the stoichiometry achieved by cotransfection of different vectors comprising the five subunits gH, gL, UL128, UL130 and UL131, but it is also superior to the stoichiometry achieved by a single vector comprising all five subunits, but each in a different open reading frame. Surprisingly, the above described preferred design of the vector according to the present invention with the two ORFs as described above results in a particularly balanced expression of the subunits gH, gL, UL128, UL130 and UL131, without excess of the gH/gL dimer and without multimers, i.e. in the assembled complex every subunit is present exactly once. The pentameric complex showing such a 1:1:1:1:1 stoichiometry of the subunits gH, gL, UL128, UL130 and UL131 provides all antigenic sites (cf. FIG. 5), i.e. as many antigenic sites as possible, which is advantageous for the production of antibodies. As an underlying mechanism the present inventors assume—without being bound to any theory—that it may be advantageous if the gH/gL subunits are first associated and then chaperoned by the subunits UL128, UL130 and UL131, whereby the preferred vector as described above appears to result in such an assembly.

Alternatively, the first open reading frame may encode one of SEQ ID NOs 3, 7 or 11 or sequence variants thereof and, both, SEQ ID NOs 21 and 25 or sequence variants thereof, while the second open reading frame encodes SEQ ID NOs 3 and 7 or sequence variants thereof or 3 and 11 or sequence variants thereof or 7 and 11 or sequence variants thereof, respectively. However, the nucleotide sequences encoding the five immunogenic components gH, gL, UL128, UL130 and UL131 may be also arranged in any other way in the two open reading frames, e.g. with a first ORF comprising a nucleotide sequence encoding gH, gL and one of UL128, UL130 and UL131 and a second ORF comprising a nucleotide sequence encoding the other two of UL128, UL130 and UL131.

Accordingly, the inventive vector preferably comprises at least two transcription units, each of which comprises an ORF, operably linked to a promoter. Each of the ORFs may further comprise e.g. a 5′ start codon and may encode two or more HCMV viral glycoproteins, such as e.g. gH (e.g. SEQ ID NO: 21), gL (e.g. SEQ ID NO:25), pUL128 (e.g. SEQ ID NO:3), pUL130 (e.g. SEQ ID NO:7), or pUL131 (e.g. SEQ ID NO:11), or sequence variants thereof. However, it is preferred that the vector according to the present invention does not encode any CMV peptide or protein other than the five subunits of the hCMV pentameric complex, namely gH, gL, UL128, UL130 and UL131.

While the immunogenic components as defined above are encoded by the inventive vector, the inventive vector may also encode one or more immunogenic component(s) other than those mentioned above. Moreover, the inventive vector, preferably in an inventive gene expression system, may also comprise nucleotide sequences encoding one or more of the following further functional components: signal peptide sequence(s), linking sequence(s), tag sequence(s), sequences comprising a cleavage site and sequences comprising sites for ribosomal skipping.

According to a preferred embodiment, the at least one ORF of the inventive expression system, in particular the first and/or the second ORF of the vector of the inventive expression system, may further comprise one or more a nucleotide sequences encoding amino acid sequences, which reflect ribosomal skipping sites. Preferably, a nucleotide sequence encoding a ribosomal skipping site is a nucleotide sequence encoding the amino acid sequence Asp-Val/Ile-Glu-X-Asn-Pro-Gly-Pro (SEQ ID NO: 56), wherein X may be any amino acid. Typically, such ribosomal skipping sites are located in between nucleotide sequences encoding for the immunogenic components such that the immunogenic components are provided as separate entities in the course of mRNA translation. The underlying mechanism is based on non-formation of a covalent linkage between two amino acids, i.e. G (Gly) and P (Pro) during mRNA translation. Accordingly, the mRNA translation is not interrupted by the non-formation of a covalent bond between the Gly/Pro, but rather proceeds without stopping the ribosomal activity on the mRNA. In particular, the ribosomes do not form a peptide bond between these amino acids, if a sequence pattern Asp-Val/Ile-Glu-X-Asn-Pro-Gly≠Pro occurs in a peptide sequence. Non-formation of a covalent bond occurs between the C-terminal Gly-Pro position of the above amino acid stretch. The vector of the present invention preferably provides for such a self-processing sequence by preferably locating a nucleotide sequence encoding for the above sequence motif between at least two of the nucleotide sequences encoding for an immunogenic component as defined above, preferably the underlying nucleotide sequence of the first and/or second open reading frame encodes for such a self-processing peptide between all of the immunogenic components as defined above. By such a self-processing sequence motif, it becomes possible to provide one open reading frame containing two or more nucleotide sequences encoding for an immunogenic component as defined above, allowing, however, to still produce separate entities of the immunogenic components as the result of mRNA-translation. Thereby, the invention allows to ensure strict compliance with a 1:1:1:1:1 stoichiometry and is not dependent on the less precise (in terms of the intracellular ratio of the immunogenic components) production of immunogenic components resulting from polycistronic gene products, which dependent on the activity of the ribosomes on ribosomal entry site (IRES).

More preferably, the inventive vector may comprise a nucleotide sequence encoding SEQ ID NO:5 (T2A) and SEQ ID NO:9 (F2A), SEQ ID No: 23 (P2A) (or e.g. its variants SEQ ID No: 27 or 29) or sequence variants thereof. SEQ ID NO: 5 and SEQ ID NO: 9 are encoded by nucleotide sequences SEQ ID Nos 6 and 10, SEQ ID No 23, 27 and 29 are encoded by SEQ ID No. 24, 28 and 30 or sequence variants thereof. They all reflect 2A self-processing peptides, namely T2A, F2A and P2A, respectively, of the Foot-and-Mouth Disease virus. Preferably, the nucleotide sequences encoding the amino acid sequences according to SEQ ID NO: 5 and SEQ ID NO: 9, in particular the nucleotide sequences according to SEQ ID No 6 and 10 (or their sequence variants), are located in between the nucleotide sequences coding for the immunogenic components, in particular in between UL128 and UL130 and/or in between UL130 and UL131. Thereby, it is understood, that the nucleotide sequences encoding UL128, UL130, and UL131 are all located within one single ORF. The nucleotide sequence encoding SEQ ID No 23 (or its sequence variants), in particular the nucleotide sequences according to SEQ ID NO: 24 (or sequence variants thereof), is preferably located between the nucleotide sequences encoding gH and gL, e.g. by another open reading frame, since it is understood that the nucleotide sequences encoding gH and gL are also located within one single ORF. In any case, each of these self-processing nucleotide sequences may be positioned between any of the nucleotide sequences of the immunogenic components. For example, the inventive vector of the inventive gene expression system comprises a first and/or a second ORF, which comprises at least one or more nucleotide sequences encoding a ribosomal skipping site having an amino acid sequence according to SEQ ID NO: 56, in particular the first and/or the second ORF comprises at least one or more nucleotide sequences selected from the group comprising SEQ ID NO:6 and/or SEQ ID NO:10 and/or SEQ ID NO:24 and/or SEQ ID NO:28 and/or SEQ ID NO:30 or sequence variants thereof. According to a preferred embodiment, the inventive vector of the inventive gene expression system comprises a first ORF, which comprises at least one nucleic acid sequence according to SEQ ID NO:24 and/or SEQ ID NO:28 and/or SEQ ID NO:30 or sequence variants thereof and the second ORF comprises at least one nucleotide sequence according to SEQ ID No: 6 and/or 10 or sequence variants thereof.

If the vector according to the present invention comprises at least one ORF, which comprises more than one nucleotide sequences encoding a HCMV pentameric glycoprotein complex subunit—e.g. a first ORF comprising a nucleotide sequence encoding gH and a nucleotide sequence encoding gL or sequence variants thereof and a second ORF comprising a nucleotide sequence encoding UL128, a nucleotide sequence encoding UL130 and a nucleotide sequence encoding UL131 or sequence variants thereof—it is preferred that within each ORF, which comprises more than one nucleotide sequences encoding a HCMV pentameric glycoprotein complex subunit, a nucleotide sequences encoding a ribosomal skipping site, e.g. a nucleotide sequences encoding a ribosomal skipping site having an amino acid sequence according to SEQ ID NO: 56, e.g. a nucleotide sequence encoding SEQ ID NO:5 (T2A), SEQ ID NO:9 (F2A), or SEQ ID No: 23 (P2A) or its variants SEQ ID No: 27 or 29 or sequence variants thereof, is located between each of two nucleotide sequences encoding a HCMV pentameric glycoprotein complex subunit, e.g. a different HCMV pentameric glycoprotein complex subunit. Thus, on such a preferred vector within each ORF each two “adjacent” nucleotide sequences encoding a CMV pentamer subunit are separated by a nucleotide sequences encoding a ribosomal skipping site, e.g. a nucleotide sequences encoding a ribosomal skipping site having an amino acid sequence according to SEQ ID NO: 56, e.g. a nucleotide sequence encoding SEQ ID NO:5 (T2A), SEQ ID NO:9 (F2A), or SEQ ID No: 23 (P2A) or its variants SEQ ID No: 27 or 29 or sequence variants thereof.

According to a preferred embodiment, the inventive vector may comprise one or more additional nucleotide sequence(s), which encode(s) a signal peptide, in particular a signal peptide, which allows the peptides to be produced in the mammalian cell to be secreted to the extracellular environment for a ready-to-go protein complex harvesting process. Among such signal peptides, IgG signal peptide sequences, e.g. a human or murine IgG signal peptide, such as e.g. SEQ ID NO:19 may be used. In this context, it is particularly preferred that the sequence encoding the gH signal peptide is replaced by a sequence encoding the IgG leader sequence, e.g. by SEQ ID NO: 19 or sequence variants thereof. However, also any other replacement of this gH signal peptide by a signal peptide sequence is preferred. Moreover, it is also preferred that—e.g. in addition to a replacement of the gH signal peptide as described above—the sequence encoding the UL128 signal peptide is replaced by a sequence encoding the IgG leader sequence, e.g. by SEQ ID:NO 19 or sequence variants thereof. However, also any other replacement of this UL128 signal peptide by a signal peptide sequence is preferred. Moreover, any other addition of a signal peptide sequence may occur. For example, the underlying nucleotide sequences encoding such signal peptide sequences may be located such that each immunogenic component, if translated as a separate entity, e.g. due to the incorporation of self-processing ribosomal skipping sites in the open reading frame's nucleotide sequence encompasses such a signal peptide sequence. In this case, the signal peptide is preferably identical for each immunogenic component and preferably identically located, e.g. at all at the 5′ terminus of the nucleotide sequence for the immunogenic component. Preferably, such a signal peptide sequence is provided, preferably at the 5′ or the 3′ terminus of the immunogenic components.

The term “identical” as used herein means that each “identical” signal peptide is of the same type, for example each identical signal peptide is a mouse IgG signal peptide or each identical signal peptide is a human IgG signal peptide or each identical signal peptide is any other specified signal peptide of the same type. More preferably, each “identical” signal peptide has the same amino acid sequence, e.g. SEQ ID NO:19; even more preferably, each “identical” signal peptide is encoded by the same nucleotide sequence, e.g. SEQ ID NO:20. In particular, the term “identical” as used herein does imply any number of encoded signal peptides (or number of nucleic acid sequences encoding a signal peptide) contained in the vector. That means in particular that the term “identical” as used herein does not necessarily imply that only one single signal peptide (or only one single nucleotide sequence encoding a signal peptide) exists in a vector according to the present invention wherein all signal peptides (or nucleotide sequences encoding a signal peptide) are identical. Instead, a vector according to the present invention, wherein (all) the encoded signal peptides (or (all) the nucleotide sequences encoding a signal peptide) are identical, may have one or more signal peptides (or nucleotide sequences encoding a signal peptide) of the same type as described above. For example, a vector having a nucleotide sequence encoding a first signal peptide and a nucleotide sequence encoding a second identical signal peptide has preferably (at least) two nucleotide sequences encoding signal peptides of the same type, preferably of the same sequence as described above.

Moreover, the inventive vector may further comprise one or more nucleotide sequences coding for one or more tag peptide(s), cleavage sites and/or linker peptides. Such tag peptide, cleavage site or linker peptide encoding nucleotide sequences may be positioned within the first and/or second ORF. They may be selected from e.g. one or more of a nucleotide sequence encoding a TEV cleavage site, in particular a nucleotide sequence according to SEQ ID NO:14, a nucleotide) sequence encoding a GS linker peptide, in particular a nucleotide sequence according to SEQ ID NO:16, a nucleotide sequence encoding a Strep-tag sequence, in particular a nucleotide sequence according to SEQ ID NO:18 and/or a nucleotide sequence according to SEQ ID NO: 40; and/or a nucleotide sequence encoding a His-tag sequence, in particular a nucleotide sequence according to SEQ ID NO: 42 or sequence variants thereof encoding SEQ ID NO:13, SEQ ID NO:15, SEQ ID NO:17, SEQ ID NO:39 and SEQ ID NO:41 or sequence variants thereof. They exhibit a TEV cleavage site, a GS linker, a STREP-tag and a 6×His-tag, which may e.g. be used for purification of encoded HCMV surface glycoproteins and/or of the inventive soluble protein complex. In particular, one or more nucleotide sequences may be selected from the group consisting of a nucleotide sequence encoding a TEV cleavage site, in particular a nucleotide sequence according to SEQ ID NO:14, a nucleotide sequence encoding a GS linker peptide, in particular a nucleotide sequence according to SEQ ID NO:16, a nucleotide sequence encoding a Strep-tag sequence, in particular a nucleotide sequence according to SEQ ID NO:18 and/or a nucleotide sequence according to SEQ ID NO: 40; and a nucleotide sequence encoding a His-tag sequence, in particular a nucleotide sequence according to SEQ ID NO: 42 or sequence variants thereof encoding SEQ ID NO:13, SEQ ID NO:15, SEQ ID NO:17, SEQ ID NO:39 and SEQ ID NO:41 or sequence variants thereof.

Nucleotide sequences encoding cleavage sites may incorporated into the open reading frame to e.g. avoid the use of self-processing skipping sites. Such cleavage sites allow to—posttranslationally—cleave the protein translated from the one or more open reading frames, in particular a protein, which comprising two or more of the immunogenic components. By such a protein cleavage, e.g. by a peptidase or proteinase, the covalently linked immunogenic components comprised in the translated gene product (one single chain) is processed into fragments, each fragment preferably comprising one immunogenic component. Accordingly, such cleavage sites are positioned within linker sequences between the immunogenic components. Another example for using cleavage sites is based on its use to specifically cleave the peptide products obtained e.g. due to ribosomal skipping such that any N- or C-terminal elongation of the immunogenic component (resulting from mRNA-translation) is cleaved off, e.g. any amino acids elongating the immunogenic component, e.g. at its C-terminus, due to the ribosomal skipping site motif. As a further example the cleavage site is preferably located adjacent to a tag, which is useful for the purification such as a 6×His-tag or a Strep-tag or tandem Strep-tag, so that the tag can be removed after purification and is thus not present in the final product to be used for vaccination. Under such circumstances, the cleavage site is preferably located close to or directly linked to the N- or C-terminal residue of the immunogenic component.

Another embodiment of the present invention provides a vector, which does not contain—between immunogenic components—any skipping or cleavage sites. Under such circumstances the nucleotide sequence of the open reading frame provides one single protein chain comprising more than immunogenic component, e.g. 2 to 5 immunogenic components as defined above, which are covalently connected, preferably via a linker chain. The complex of the invention resulting from an aggregation of each of the immunogenic components may thereby be formed by at least two (or even 5) immunogenic components, which are all covalently linked with each other.

According to a more specific preferred embodiment, the inventive vector comprises a first ORF, which comprises the first promoter and operably linked to it nucleotide sequences encoding the amino acid sequence of SEQ ID NO:19, SEQ ID NO:21, SEQ ID NO:23 and SEQ ID NO:25 or sequence variants thereof, or the nucleotide sequences encoding the amino acid sequences of SEQ ID NO:19, SEQ ID NO:35, SEQ ID NO:27 and SEQ ID NO:37 or sequence variants thereof, or the nucleic acid sequences encoding the amino acid sequences of SEQ ID NO:19, SEQ ID NO:35, SEQ ID NO:29 and SEQ ID NO:37 or sequence variants thereof, and a second ORF, which comprises a second promoter and, operably linked to it, nucleotide sequences encoding amino acid sequences according to SEQ ID NO:1, SEQ ID NO:3, SEQ ID NO:23, SEQ ID NO:7, SEQ ID NO:23, and SEQ ID NO:11 or sequence variants thereof, or operably linked to it, nucleotide sequences encoding amino acid sequences according to SEQ ID NO:19, SEQ ID NO:3, SEQ ID NO:23, SEQ ID NO:7, SEQ ID NO:23, SEQ ID NO:11, SEQ ID NO:13, SEQ ID NO:15, SEQ ID NO:17 and SEQ ID NO:41 or sequence variants thereof or the nucleic acid sequences encoding the amino acid sequences of SEQ ID NO:1, SEQ ID NO:3, SEQ ID NO:5, SEQ ID NO:7, SEQ ID NO:9 and SEQ ID NO:11 or sequence variants thereof, or the nucleic acid sequences encoding the amino acid sequences of SEQ ID NO:1, SEQ ID NO:3, SEQ ID NO:5, SEQ ID NO:7, SEQ ID NO:9, SEQ ID NO:11, SEQ ID NO:13 and SEQ ID NO: 41 or sequence variants thereof. Thereby it is preferred that the positioning of the nucleotide sequences encoding the above described amino acid sequences within the first and/or the second ORF is in the same order as mentioned above, i.e. in N-C-terminal direction of the peptides or in 5′-3′ direction for the encoding nucleotide sequences.

More specifically, the inventive vector may comprise additional sequences such as e.g. the nucleotide sequence encoding SEQ ID NO:1, which reflects the amino acid sequence of a viral signal peptide, or e.g. the vector of the inventive gene expression system may comprise in a second ORF sequence variants of, pUL130, pUL131, such as e.g. the nucleotide sequences encoding SEQ ID NO:31 and/or SEQ ID NO:33, SEQ ID NO:37, which may be present in any order as described below, with the exception of SEQ ID NO:19. According to a more specific embodiment, the vector of the inventive gene expression system comprises a second ORF, which comprises operably linked the nucleic acid sequences encoding SEQ ID NO:1, SEQ ID NO:3, SEQ ID NO:23, SEQ ID NO:7, SEQ ID NO:9, SEQ ID NO:11, SEQ ID NO:13, SEQ ID NO:15 and SEQ ID NO:17, or the nucleic acid sequences encoding SEQ ID NO:19, SEQ ID NO:3, SEQ ID NO:27, SEQ ID NO:31, SEQ ID NO:27 and SEQ ID NO:33, or the nucleic acid sequences encoding SEQ ID NO:19, SEQ ID NO:3, SEQ ID NO:29, SEQ ID NO:31, SEQ ID NO:29 and SEQ ID NO:33, or the nucleic acid sequences encoding SEQ ID NO:19, SEQ ID NO:3, SEQ ID NO:27, SEQ ID NO:31, SEQ ID NO:27, SEQ ID NO:33, SEQ ID NO:13, SEQ ID NO:15, SEQ ID NO:39 and SEQ ID NO:41, or the nucleic acid sequences encoding SEQ ID NO:19, SEQ ID NO:3, SEQ ID NO:29, SEQ ID NO:31, SEQ ID NO:29, SEQ ID NO:33, SEQ ID NO:13, SEQ ID NO:15, SEQ ID NO:39 and SEQ ID NO:41, or the nucleic acid sequences encoding SEQ ID NO:19, SEQ ID NO:3, SEQ ID NO:27, SEQ ID NO:31, SEQ ID NO:27, SEQ ID NO:33, SEQ ID NO:13, SEQ ID NO:15, SEQ ID NO:39, or the nucleic acid sequences encoding SEQ ID NO:19, SEQ ID NO:3, SEQ ID NO:29, SEQ ID NO:31, SEQ ID NO:29, SEQ ID NO:33, SEQ ID NO:13, SEQ ID NO:15, and SEQ ID NO:39.

More specifically, the inventive vector comprises a second ORF, which comprises a nucleotide sequence encoding SEQ ID NO:3, SEQ ID NO:7, and SEQ ID NO:11 or sequence variants thereof.

According to one embodiment, the first ORF and/or second ORF of the inventive vector comprise the nucleotide sequences encoding SEQ ID NO:19, SEQ ID NO:3, SEQ ID NO:27, SEQ ID NO:31, SEQ ID NO:27, SEQ ID NO:33, SEQ ID NO:19, SEQ ID NO:35, SEQ ID NO:27 and SEQ ID NO:37 or sequence variants thereof.

More specifically, the first ORF and/or second ORF of the inventive vector comprises the nucleotide sequences encoding SEQ ID NO:19, SEQ ID NO:3, SEQ ID NO:29, SEQ ID NO:31, SEQ ID NO:29, SEQ ID NO:33, SEQ ID NO:19, SEQ ID NO:35, SEQ ID NO:29 and SEQ ID NO:37 or sequence variants thereof.

In one embodiment, the first ORF and/or second ORF of the inventive vector comprise the nucleotide sequences encoding SEQ ID NO:19, SEQ ID NO:3, SEQ ID NO:27, SEQ ID NO:31, SEQ ID NO:27, SEQ ID NO:33, SEQ ID NO:13, SEQ ID NO:15, SEQ ID NO:39, SEQ ID NO:19, SEQ ID NO:35, SEQ ID NO:27 and SEQ ID NO:37 or sequence variants thereof.

In one embodiment, the first ORF and/or second ORF of the inventive vector comprise the nucleotide sequences encoding SEQ ID NO:19, SEQ ID NO:3, SEQ ID NO:29, SEQ ID NO:31, SEQ ID NO:29, SEQ ID NO:33, SEQ ID NO:13, SEQ ID NO:15, SEQ ID NO:39, SEQ ID NO:19, SEQ ID NO:35, SEQ ID NO:29 and SEQ ID NO:37 or sequence variants thereof.

In a second aspect, the present invention provides for a gene expression system, which comprises at least one mammalian cell and the inventive vector, as described above, for expressing HCVM glycoproteins in said mammalian cell. Such a gene expression system may be provided as a kit comprising the at least one mammalian cell, e.g. a mammalian cell culture of such mammalian cells (e.g. as a suspension of cells in a cell culture medium) and, separately, at least one vector according to the invention. Or, the inventive gene expression system is provided by at least one mammalian cell, preferably as a mammalian cell culture as mentioned above, wherein the cells are transfected by the inventive vector. In this context it is particularly preferred that the mammalian cells are stably transfected by the inventive vector, for example the cells may be nucleofected by the inventive vector. Accordingly, the present invention also provides a stable cell line secreting a HCMV pentamer comprising amino acid sequences according to SEQ ID NO:3, SEQ ID NO:7, SEQ ID NO:11, SEQ ID NO:21 and SEQ ID NO:25 or sequence variants thereof, wherein said stable cell line is obtainable by transfection, preferably nucleofection, of at least one mammalian cell with a vector according to the present invention.

By such an inventive gene expression system a yield, which is several folds higher than that of conventional expression systems using adenoviruses or transfection with multiple plasmids can be achieved. Therefore, a high quantity of the HCMV pentameric protein complex can be provided, which is very useful for example in large scale production of the respective vaccine.

According to a preferred embodiment, the at least one mammalian cell of the inventive gene expression system may be any appropriate mammalian producer cell, but is preferably selected from the group comprising BHK, DUXB11, CHO-DG44, CHO-K1, CHO-K1SV, CHO-S, CHO-DXB11, CHO-K1SV GS knock-out (CHO-K1SV KO), CAP, PER.C6, NS0, Sp2/0, HEK293 T, HEK 293-F, HEK 6E, HEK293 EBNA, CAP-T, HELA, CVI, COS, R1610, BALBC/3T3, HAK, BFA-1c1BPT, RAJI, HT-1080 and HKB-11. In a more preferred embodiment, the at least one mammalian cell of the inventive gene expression system is selected from the group comprising CHO-DG44, CHO-K1, CHO-K1SV, CHO-S, CHO-DXB11 and CHO-K1SV GS knock-out (CHO-K1SV KO). Most preferred are CHO-K1SV and CHO-K1SV GS knock-out (CHO-K1SV KO) cells.

In a third aspect, the present invention provides for a soluble protein complex obtainable by the inventive gene expression system or by the inventive stable cell line, which preferably comprises the subunits gH, gL, UL128, UL130 and UL131, preferably the respective amino acid sequences according to SEQ ID NO:3, SEQ ID NO:7, SEQ ID NO:11, SEQ ID NO:21 and SEQ ID NO:25 or sequence variants thereof. Preferably, the complex comprises one of each of the above 5 amino acid sequences in a 1:1:1:1:1 stoichiometry and, optionally, further components. Preferably, the complex comprises no more than one of each of the above 5 amino acid sequences, while other amino acid sequences (not comprising the immunogenic components as mentioned above) may be comprised in the inventive soluble complex.

Each of the above 5 immunogenic components, preferably the amino acid sequences according to SEQ ID NO:3, SEQ ID NO:7, SEQ ID NO:11, SEQ ID NO:21 and SEQ ID NO:25 or sequence variants thereof, may be provided as separate entity within the complex or may be provided by covalently coupling two or more (2 to 5) of these amino acid sequences with or without e.g. peptide linker sequences (of a length of e.g. 1 to 100 amino acids, preferably, 5 to 50, more preferably 5 to 30, most preferably 5 to 20 amino acids). Accordingly, the 5 amino acid sequences mentioned above may be provided as five, four, three, two or one single separate entity within the soluble protein complex. However, it is preferred that the five hCMV pentamer subunits as described herein are provided as five separate entities within the soluble protein complex, since covalent coupling of two or more (2 to 5) of these amino acid sequences with or without e.g. a peptide linker sequence may result in poorer recognition of the antigenic sites on the coupled subunits by an antibody, in particular by an antibody specifically binding to the relevant antigenic site.

In one embodiment, the present invention provides for a soluble protein complex obtainable by the inventive gene expression system, wherein the protein complex comprises the amino acid sequences of gH, gL, UL128, UL130, and UL131, in particular according to SEQ ID No: 3, SEQ ID No: 7, SEQ ID No: 11, SEQ ID No: 21 and SEQ ID No: 25 or sequence variants thereof. As described above, these amino acid sequences reflecting the immunogenic components may be provided as one single protein chain, e.g. by covalently linking the two to five immunogenic components with each other. Preferably, however, the above immunogenic components are separate entities, which are not covalently linked to each other and aggregate via non-covalent interaction, e.g. hydrogen bonding, van der Waals interaction etc., to form complexes containing one single polypeptide representing and comprising the individual immunogenic component. As disclosed above, the formation of single polypeptides containing the immunogenic components of gH, gL, UL128, UL130, and UL131 may be achieved by RNA skipping due to RNA skipping sites located between two such immunogenic components or by posttranslational protein cleavage. However, the preferably five separate polypeptides (each containing a distinct of the above immunogenic components) forming the complex may contain each additional amino acid sequences, in particular at their N- and/or C-termini. These additional sequences arise from nucleotide sequence elements within the open reading frame(s) of the inventive vector. E.g. signal peptides may be encoded by the nucleotide sequence of the open reading frame thereby elongating the immunogenic components e.g. at their termini. Also linker sequences (or portions thereof) may elongate the immunogenic component. That holds in case of cleavage or self-processing of full length amino acid sequence in the course of translation or posttranslation as well. Accordingly, the 5′ upstream immunogenic component (according to its location in the open reading frame) may contain at its C-terminal end the N-terminal sequence of e.g. a linker element or N-terminal sequence of a self-processing motif, while the downstream immunogenic component may contain at its N-terminal end the C-terminal sequence of e.g. a linker sequence or of the self-processing element. Accordingly, the amino acid sequence according to the nucleotide sequence of the open reading frame is typically reflected by the soluble protein complex. However, e.g. linker sequences connecting the immunogenic components at the nucleotide sequence level may be cleaved at the protein level and may then be allocated by its N-terminal and C-terminal portions to e.g. the terminal sequences of (distinct) polypeptides comprising individually the immunogenic components as elements of the inventive soluble protein.

In general, with regard to embodiments providing for the soluble protein complex it is of note that the amino acid sequence comprising e.g. a ribosomal skipping site, e.g. SEQ ID NOs: 5, 9, 23, 27, and 29 and 56 are separated due to the ribosomal skipping, e.g. between the GLY and the Pro residue. Thus, the respective amino acid sequences are not provided by in the usual continuous structure, but are provided separately as two portions linked to two distinct polypetides, e.g. immunogenic compounds (both of which forming part of the inventive soluble complex).

The soluble protein complex may comprise SEQ ID NO:19, SEQ ID NO:21, SEQ ID NO:23, SEQ ID NO:25, SEQ ID NO:1, SEQ ID NO:3, SEQ ID NO:5, SEQ ID NO:7, SEQ ID NO:9, SEQ ID NO:11, SEQ ID NO:13, SEQ ID NO:15, and SEQ ID NO:17 or sequence variants thereof. More specifically, the soluble protein complex according to the invention may comprise the amino acid sequences according to SEQ ID NO:19, SEQ ID NO:35, SEQ ID NO:27, SEQ ID NO:37, SEQ ID NO:3, SEQ ID NO:27, SEQ ID NO:31, SEQ ID NO:27, and SEQ ID NO:33 or sequence variants thereof. Alternatively, the inventive soluble protein complex may comprise the amino acid sequences according to SEQ ID NO:35, SEQ ID NO:29, SEQ ID NO:37, SEQ ID NO:19, SEQ ID NO:3, SEQ ID NO:29, SEQ ID NO:31, SEQ ID NO:29 and SEQ ID NO:33 or sequence variants thereof.

According to one embodiment, the inventive soluble protein complex may comprise the amino acid sequences according to SEQ ID NO:19, SEQ ID NO:35, SEQ ID NO:27, SEQ ID NO:37, SEQ ID NO:19, SEQ ID NO:3, SEQ ID NO:27, SEQ ID NO:31, SEQ ID NO:27, SEQ ID NO:33, SEQ ID NO:13, SEQ ID NO:15, SEQ ID NO:39 and SEQ ID NO:41 or sequence variants thereof.

More specifically, the inventive soluble protein complex may comprise the amino acid sequences according to SEQ ID NO:19, SEQ ID NO:35, SEQ ID NO:29, SEQ ID NO:37, SEQ ID NO:19, SEQ ID NO:3, SEQ ID NO:29, SEQ ID NO:31, SEQ ID NO:29, SEQ ID NO:33, SEQ ID NO:13, SEQ ID NO:15, SEQ ID NO:39 and SEQ ID NO:41 or sequence variants thereof.

According to one embodiment, the inventive soluble protein complex may comprise the amino acid sequences according to SEQ ID NO:19, SEQ ID NO:35, SEQ ID NO:27, SEQ ID NO:37, SEQ ID NO:19, SEQ ID NO:3, SEQ ID NO:27, SEQ ID NO:31, SEQ ID NO:27, SEQ ID NO:33, SEQ ID NO:13, SEQ ID NO:15 and SEQ ID NO:39 or sequence variants thereof.

More specifically, the inventive soluble protein complex may comprise the amino acid sequences according to SEQ ID NO:19, SEQ ID NO:35, SEQ ID NO:29, SEQ ID NO:37, SEQ ID NO:19, SEQ ID NO:3, SEQ ID NO:29, SEQ ID NO:31, SEQ ID NO:29, SEQ ID NO:33, SEQ ID NO:13, SEQ ID NO:15, SEQ ID NO:39 or sequence variants thereof.

According to a preferred embodiment, the inventive soluble protein complex may comprise the amino acid sequence according to SEQ ID NO:43, or SEQ ID NO:45, or SEQ ID NO:47, or SEQ ID NO:49 or sequence variants thereof.

According to one embodiment, the present invention provides a soluble protein complex according to the invention (or alternatively the vector of the invention) for use as a vaccine.

In a fourth aspect, the present invention provides for a vaccine composition, which comprises the inventive soluble protein complex and, optionally, one or more additional pharmaceutically active components and further, optionally, one or more pharmaceutically inactive components, in particular a vehicle, carrier, preservative etc. In particular, the inventive vaccine composition comprises one or more adjuvants selected from the group comprising mineral salts, surface-active agents, microparticles, cytokines, hormones, detergents, squalene, Alum, polyanions or polyacrylics. Preferably, the adjuvant comprised in inventive vaccine composition is selected from the group consisting of Freud's incomplete or complete adjuvant, Alum, Ribi (Monophosphoryl lipid A, MPL), and MF59.

In particular, the inventive vaccine composition is obtainable by the use of an inventive vector or, more specifically, an inventive gene expression system or an inventive stable cell line. As mentioned above, the vaccine composition according to the invention elicits predominantly neutralizing antibodies and has thus a very high specific activity, which is due to the HCMV pentameric glycoprotein complex having a proper structure due to the design of the inventive vector.

In particular, the vaccine according to the present invention has thus a high proportion of the HCMV pentameric glycoprotein complex having a proper structure, i.e. preferably more than 80%, more preferably more than 90%, even more preferably more than 95% and most preferred more than 99% of each of the HCMV pentameric glycoprotein complex subunits gH, gL, UL128, UL130 and UL131 contained in the vaccine are assembled in a HCMV pentameric glycoprotein complex having the proper structure, which preferably reflects a 1:1:1:1:1 stoichiometry of these subunits and whereby the subunits preferably assume their native structure in the complex so that the HCMV pentameric glycoprotein complex preferably assumes its native structure, which is detectable e.g. by NMR spectroscopy methods. This enables a highly specific antibody response and ensures thus a high specific activity of the vaccine.

Additionally or according to the alternative embodiment, the vector of the invention may be formulated as a vaccine composition and may be injected into the human as well. The protein complex is—under such conditions—produced in vivo and secreted from the in vivo producer cells.

In a preferred embodiment, the inventive vaccine composition may be a liquid formulation, or a solid formulation, e.g. a lyophilized formulation. If provided in a lyophilized form, which is preferred in view of transportation, stability, etc., it is preferably dissolve the lyophilized form prior to its administration.

The inventive vaccine composition, in particular when provided in liquid form, comprises in particular a carrier or vehicle, The carrier or vehicle is typically an aqueous solution, potentially being composed of a mixture of water and another organic solvent being miscible with water, e.g. ethanol, DMSO etc. It may further be a buffered solution comprising a buffer preferably selected from the group of phosphate buffer, Na-acetate buffer, Tris buffer, MOPS buffer. Preferably, the buffer is a phosphate buffer. More specifically, the buffer of the inventive vaccine composition buffers the vaccine composition at a pH range of about pH 7-9, preferably between 7 and 8. Furthermore, the vaccine composition is preferably dissolved in a carrier which is essentially isotonic.

The vaccine composition according to the present invention is disclosed in particular for its use in the vaccination of a human, typically against HCMV infections, for prophylactic and/or therapeutic application, preferably for prophylactic use.

In a fifth aspect, the present invention provides for a process of preparing a vaccine, in particular a vaccine composition, according to any one of the above embodiments. The process for preparing a vaccine composition according to the present invention comprises the following steps:
(a) Preparation of a vector according to any of claims 1 to 38 is prepared;
(b) Transfection of a mammalian producer cell with the vector prepared in step (a);
(c) Harvesting a HCMV pentamer comprising the amino acid sequences according to SEQ ID NO:3, SEQ ID NO:7, SEQ ID NO:11, SEQ ID NO:21 and SEQ ID NO:25 or sequence variants thereof from the mammalian producer cell;
(d) Optionally purification of the HCMV pentamer harvested in step (c); and
(e) Formulation of the harvested and optionally purified HCMV pentamer as a liquid or solid formulation.

According a sixth aspect, the present invention provides for a nucleic acid, which comprises nucleotide sequences encoding SEQ ID NO:1, SEQ ID NO:3, SEQ ID NO:7, SEQ ID NO:11, SEQ ID NO:21, and SEQ ID NO:25 or sequence variants thereof, or nucleotide sequences encoding SEQ ID NO:1, SEQ ID NO:3, SEQ ID NO:7, SEQ ID NO:11, SEQ ID NO:21, SEQ ID NO:25, SEQ ID NO:13, SEQ ID NO:15 and SEQ ID NO:41 or sequence variants thereof.

According to a more preferred embodiment, the nucleic acid according to the invention further comprises nucleotide sequences encoding SEQ ID NO:5 and/or SEQ ID NO:9 and/or SEQ ID NO:23, and/or SEQ ID NO:27, and/or SEQ ID NO:29 or sequence variants thereof, preferably comprising SEQ ID NO:23 and/or SEQ ID NO:27 and/or SEQ ID NO:29 or sequence variants thereof.

More specifically, the inventive nucleic acid further comprises operably linked in 5′ to 3′ direction the nucleic acid sequences encoding SEQ ID NO:1, SEQ ID NO:3, SEQ ID NO:5, SEQ ID NO:7, SEQ ID NO:9 SEQ ID NO:11, SEQ ID NO:13, SEQ ID NO:15, SEQ ID NO:17, SEQ ID NO:19, SEQ ID NO:21, SEQ ID NO:23 and SEQ ID NO:25 or sequence variants thereof.

According to an even more preferred embodiment, the inventive nucleic acid comprises operably linked in 5′ to 3′ direction the nucleic acid sequences encoding SEQ ID NO:19, SEQ ID NO:3, SEQ ID NO:27, SEQ ID NO:31, SEQ ID NO:27, SEQ ID NO:33, SEQ ID NO:19, SEQ ID NO:35, SEQ ID NO:27 and SEQ ID NO:37 or sequence variants thereof.

More specifically, the inventive nucleic acid comprises operably linked in 5′ to 3′ direction the nucleic acid sequences encoding SEQ ID NO:19, SEQ ID NO:3, SEQ ID NO:29, SEQ ID NO:31, SEQ ID NO:29, SEQ ID NO:33, SEQ ID NO:19, SEQ ID NO:35, SEQ ID NO:29 and SEQ ID NO:37 or sequence variants thereof.

According to one embodiment, the nucleic acid according to the invention comprises operably linked in 5′ to 3′ direction the nucleic acids encoding SEQ ID NO:19, SEQ ID NO:3, SEQ ID NO:27, SEQ ID NO:31, SEQ ID NO:27, SEQ ID NO:33, SEQ ID NO:13, SEQ ID NO:15, SEQ ID NO:39, SEQ ID NO:41, SEQ ID NO:19, SEQ ID NO:35, SEQ ID NO:27 and SEQ ID NO:37 or sequence variants thereof.

According a further embodiment, the nucleic acid according to the invention comprises operably linked in 5′ to 3′ direction the nucleic acids encoding SEQ ID NO:19, SEQ ID NO:3, SEQ ID NO:29, SEQ ID NO:31, SEQ ID NO:29, SEQ ID NO:33, SEQ ID NO:13, SEQ ID NO:15, SEQ ID NO:39, SEQ ID NO:41, SEQ ID NO:19, SEQ ID NO:35, SEQ ID NO:29 and SEQ ID NO:37 or sequence variants thereof.

According to a further embodiment, the nucleic acid according to the invention comprises operably linked in 5′ to 3′ direction the nucleic acids encoding SEQ ID NO:19, SEQ ID NO:3, SEQ ID NO:27, SEQ ID NO:31, SEQ ID NO:27, SEQ ID NO:33, SEQ ID NO:13, SEQ ID NO:15, SEQ ID NO:39, SEQ ID NO:19, SEQ ID NO:35, SEQ ID NO:27 and SEQ ID NO:37 or sequence variants thereof.

In one embodiment, the nucleic acid according to the invention comprises operably linked in 5′ to 3′ direction the nucleic acids encoding SEQ ID NO:19, SEQ ID NO:3, SEQ ID NO:29, SEQ ID NO:31, SEQ ID NO:29, SEQ ID NO:33, SEQ ID NO:13, SEQ ID NO:15, SEQ ID NO:39, SEQ ID NO:19, SEQ ID NO:35, SEQ ID NO:29 and SEQ ID NO:37 or sequence variants thereof.

In one embodiment, the inventive nucleic acid comprises the nucleotide sequence encoding SEQ ID NO:43, or SEQ ID NO:45, or SEQ ID NO:47, or SEQ ID NO:49 or sequence variants thereof.

In one embodiment, the present invention pertains to the use of a nucleic acid according to the invention in a process according to any one of the above embodiments.

In a seventh aspect, the present invention provides for a mammalian cell, e.g. a CHO cell, as a mammalian producer cell, for use in a process for the preparation of a vaccine, wherein the mammalian producer cell comprises the inventive vector and/or the inventive nucleic acid according to any one of the above embodiments. The process for preparing a vaccine composition according to the invention is typically composed of the following steps: (a) the vector according to the invention is prepared, (b) a mammalian producer cell, e.g. a CHO cell, is transfected by the vector as provided by to (a) by an in vitro step, (c) the soluble protein complex according to the invention is harvested from the mammalian producer cell, preferably after the protein complex is secreted from the producer cell into the cell environment. The harvesting is carried by appropriate techniques, e.g. be chromatographic methods. The complex harvested according to (c) may optionally be further purified, and (e) the harvested and optionally purified soluble complex may thereafter be formulated as a liquid or solid formulation.

According to an eight aspect, the present invention provides for a kit of parts, which comprises the inventive vector and at least one mammalian cell, which is used as a producer cell for producing the soluble protein complex of the invention upon transfection with the vector of the invention.

In a ninth aspect the present invention provides for a method of vaccination of a human, wherein the method comprises administering to a person the inventive vaccine composition in therapeutically effective amounts. More specifically, the inventive method of vaccination of a human comprises administering 0.2 μg to about 200 μg of the inventive vaccine composition, wherein the vaccine composition is administered at least once, twice or three times over a period of time, e.g. within 2 to 6 weeks, and potentially and/or preferably parenterally, e.g. intramuscularly, intradermally, or subcutaneously. According to a more preferred embodiment, the inventive method of vaccinating a human comprises intramuscular administration of the inventive vaccine composition.

More specifically, the inventive method of vaccinating a human comprises administering the inventive vaccine composition in combination (e.g. by combined (by a single composition), or separately by subsequent or parallel administration) with one or more other HCMV vaccines. Such other HCMV vaccines may be selected from the group consisting of AD169 HCMV strain vaccines, Towne vaccine, UL130, UL131 peptide conjugate vaccines, gB-based vaccines, and/pp65 vaccine.

### BRIEF DESCRIPTION OF THE DRAWINGS

FIG. 1: Schematic representation of particularly preferred versions of the construct pentamer according to the present invention, which can be obtained by a vector according to the present invention as described herein. The scheme illustrates a set of preferred pentamer constructs (tagged or tagless) with some variations in the 2A peptides used (using P2A only with a short GS linker at the N-terminus or with a furin cleavage site), some variations in the genes boundaries (version 2 (v2) of some pentamer genes) and different strategies for the tagging (a new version of the tandem Streptag with or without an His-tag and the C terminus). The SEQ ID NOs for the amino acid sequences for each of the components are given in parentheses.

FIG. 2: Map of the inventive expression construct “pentamer2final” which was used for the nucleofection of CHO cells. “pUL128 2A 130 2A 131” denotes the relative position of the nucleotide sequences encoding for the HCMV glycoprotein UL128, UL130 and UL131. “2A” denotes the self-processing peptide P2A of the Foot-and-Mouth Disease virus.

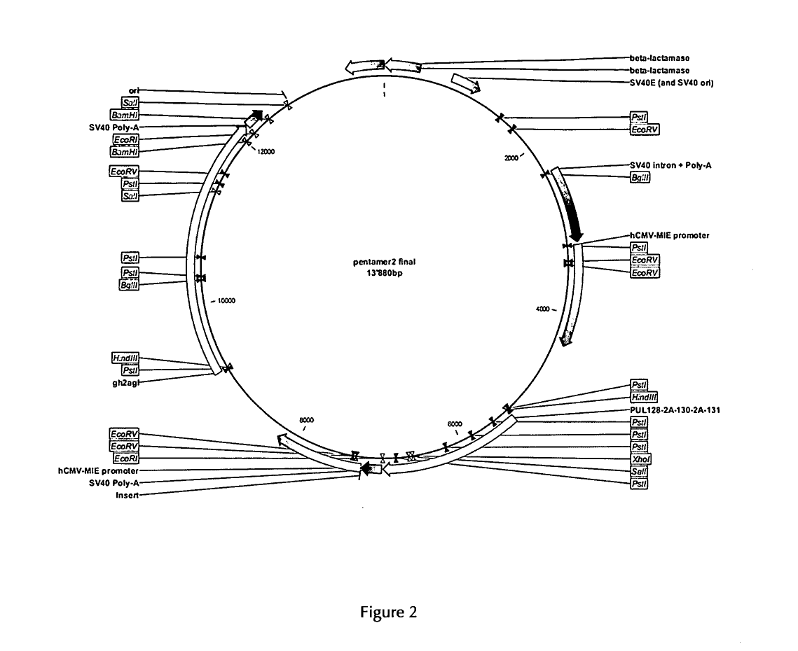

FIG. 3: Map of an inventive expression construct comprising the nucleotide sequences encoding for the HCMV glycoproteins UL128, UL130. UL131 comprises a peptide sequence encompassing a TEV cleavage site and two STREP-Tags®. “P2A”, “T2A” and “F2A” denote self-processing peptides

FIG. 4: Characterization of a soluble HCMV pentameric complex produced in CHO-K1SV cell line nucleofected with the inventive expression construct according to FIG. 2. (A) SDS-PAGE and Western blot of the inventive soluble protein complex, (B) HPLC-SEC analysis of the inventive protein complex. (C) depicts circular dichroism, far-UV spectra recorded over the wavelength range of 190 to 260 nm. The spectra in the far-UV region and secondary structures. Panel (D) depicts CD spectra measurement of thermal denaturation performed with a T-ramp of 1° C./minute.

FIG. 5: shows schematically the multiple antigenic sites on the HCMV pentamer defined by a panel of human neutralizing antibodies, which were e.g. used in a sandwich ELISA) assay as described in Example 3. The Roman numbers in parentheses indicate the different antigenic sites.

FIG. 6: shows the results of the sensitive sandwhich ELISA described in Example 3. in which serial dilutions of purified HCMV pentamer are captured by the coated human antibody 3G16 (anti-gH site I, cf. FIG. 5) followed by detection with the murinized antibodies 13H11 (anti-gH site II, cf. FIG. 5), 5A2 (anti-pUL130/131 site III, cf. FIG. 5) or 15D8 (anti-pUL128 site I, cf. FIG. 5).

FIG. 7: shows the results of nine different coating antibodies vs. the same set of antibodies as detection antibodies in the sensitive sandwich ELISA as described in Example 3 and shown in FIG. 7.

FIG. 8: A neutralization assay of HCMV using the epithelial cell line ARPE 19 as target and either a monoclonal human anti-HCMV antibody (5A2) as control or the soluble HCMV pentameric complex (cf. Example 3).

FIG. 9: Binding and neutralizing antibody titers in sera of mice immunized with different doses of the HCMV pentameric complex vaccine CHO-produced pentamer. Panels a and b show the binding antibody titers to gHgL dimer (a) and gHgLUL128L pentamer (b) measured by ELISA in the sera of mice on day +40 after immunization with different doses of the HCMV pentameric complex produced in CHO cells. Error bars show 95% CI of the geometric mean values. *P<0.05, **P<0.01. Panel c shows HCMV neutralizing serum antibody titers measured on epithelial cells (grey circle) and fibroblasts (white circles) of mice immunized with different doses of the HCMV pentameric complex. Values were normalized to the total IgG content. Panel d shows HCMV neutralizing serum antibody titers measured on epithelial cells (grey circle) and fibroblasts (white circles) of individuals 1 month or 1-2 years after natural HCMV infection or of mice immunized 40 days before with 0.2 μg HCMV pentameric complex. Each dot represents an individual mouse or individual (cf. Example 4).

FIG. 10: Neutralizing and specific antibody response elicited in Balb/c mice immunized with soluble CHO-produced HCMV pentameric complex. Panel a and b show normalized binding antibody titers for gHgL (a) and gHgLpUL128L (b) measured by ELISA in the sera of mice on day +40 after immunization with 2.5 μg of CHO-produced pentamer formulated with different adjuvants (Alum, MF59, or Ribi). Error bars show 95% CI of the geometric mean values. Panel c shows normalized neutralizing antibody titers in the sera of immunized mice measured using epithelial cells (grey dots) or fibroblasts (white dots). Panel d shows data of inhibition of monoclonal antibody binding assay (IMAB). Antibodies in sera from mice immunized with HCMV pentameric complex are superior to antibodies in sera from HCMV-infected donors to inhibit binding to HCMV proteins of monoclonal antibody specific for different epitopes in the gHgLpUL128L complex. The name and specificity of the monoclonal antibodies are shown in the x-axis. Error bars show 95% CI of the geometric mean values. Each dot corresponds to a single mouse. **P<0.01, ***P<0.001 (cf. Example 4).

FIG. 11: Characterization of mouse monoclonal antibodies from gB- and gHgLpUL128L-immunized mice. Panel a shows that the percentage of HCMV neutralizing antibodies (nAbs) among HCMV glycoprotein-binding antibodies (bAbs) is significantly higher in mice immunized with the HCMV pentameric complex compared to mice immunized with the gB vaccine. Panel b shows that a large fraction (67%) of the monoclonal antibodies induced by the HCMV pentameric vaccine bind epitopes present on the gHgL dimer and the gHgLpUL128L pentamer (cf. Example 5 and 6).

### SEQUENCE LISTING

SEQ ID NO:1: Amino acid sequence of signal peptide
SEQ ID NO:2: Nucleotide sequence encoding signal peptide
SEQ ID NO:3: Amino acid sequence of UL128
SEQ ID NO:4: Nucleotide sequence encoding UL128
SEQ ID NO:5: Amino acid sequence T2A
SEQ ID NO:6: Nucleotide sequence encoding T2A
SEQ ID NO:7: Amino acid sequence of UL130v1
SEQ ID NO:8: Nucleotide sequence encoding UL130_v1
SEQ ID NO:9: Amino acid sequence of F2A
SEQ ID NO:10: Nucleotide sequence encoding F2A
SEQ ID NO:11: Amino acid sequence of UL131v1
SEQ ID NO:12: Nucleotide sequence encoding UL131_v1
SEQ ID NO:13: Amino acid sequence of TEV site
SEQ ID NO:14: Nucleotide sequence encoding TEV site
SEQ ID NO:15: Amino acid sequence of GS linker
SEQ ID NO:16: Nucleotide sequence encoding GS linker
SEQ ID NO:17: Amino acid sequence of tandem Strep-tag_v1
SEQ ID NO:18: Nucleotide sequence encoding Strep-tag_v1
SEQ ID NO:19: Amino acid sequence of mouse IgG signal peptide
SEQ ID NO:20: Nucleotide sequence encoding mouse IgG signal peptide
SEQ ID NO:21: Amino acid sequence of gH_v1
SEQ ID NO:22: Nucleotide Sequence encoding gH_v1
SEQ ID NO:23: Amino acid sequence of P2A
SEQ ID NO:24: Nucleotide sequence encoding P2A
SEQ ID NO:25: Amino acid sequence of gL_v1
SEQ ID NO:26: Nucleotide sequence encoding gL_v1
SEQ ID NO:27: Amino acid sequence of P2A_v2
SEQ ID NO:28: Nucleotide sequence encoding P2A_v2
SEQ ID NO:29: Amino acid sequence of P2A_v3
SEQ ID NO:30: Nucleotide sequence encoding P2A_v3
SEQ ID NO:31: Amino acid sequence encoding UL130_v2
SEQ ID NO:32: Nucleotide sequence encoding UL130_v2
SEQ ID NO:33: Amino acid sequence of UL131_v2
SEQ ID NO:34: Nucleotide sequence encoding UL131_v2
SEQ ID NO:35: Amino acid sequence of gHv2
SEQ ID NO:36: Nucleotide sequence encoding gHv2
SEQ ID NO:37: Amino acid sequence of gLv2
SEQ ID NO:38: Nucleotide sequence encoding gLv2
SEQ ID NO:39: Amino acid sequence of tandem Strep-tag_v2
SEQ ID NO:40: Nucleotide sequence encoding Strep-tag_v2
SEQ ID NO:41: Amino acid sequence of 6×His tag
SEQ ID NO:42: Nucleotide sequence encoding 6×His tag
SEQ ID NO:43: Amino acid sequence of pentamer_UL128-130-131A_v1
SEQ ID NO:44: Nucleotide sequence encoding pentamer_UL128-130-131A_v1
SEQ ID NO:45: Amino acid sequence of pentamer_gH-gL_v1
SEQ ID NO:46: Nucleotide sequence encoding pentamer_gH-gL_v1
SEQ ID NO:47: Amino acid sequence of Pentamer_UL128-130-131A_v3
SEQ ID NO:48: Nucleotide sequence encoding Pentamer_UL128-130-131A_v3
SEQ ID NO:49: Amino acid sequence of pentamer_gH-gL_v3
SEQ ID NO:50: Nucleotide sequence encoding pentamer_gH-gL_v3
SEQ ID NO:51: Peptide linker sequence
SEQ ID NO:52: Peptide linker sequence
SEQ ID NO:53: Peptide linker sequence
SEQ ID NO:54: Peptide linker sequence
SEQ ID NO:55:: Peptide linker sequence
SEQ ID NO:56: Amino acid sequence motif of ribosomal skipping site

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

As used herein, “sequence variant” refers to any alteration in a reference sequence, whereby a reference sequence is any of the sequences listed in the SEQUENCE LISTING, i.e. SEQ ID NO:1 to SEQ ID NO:55. Thus, the term “sequence variant” includes nucleotide sequence variants and amino acid sequence variants.

A “nucleotide sequence variant” has an altered sequence in which one or more of the nucleotides in the reference sequence is deleted, or substituted, or one or more nucleotides are inserted into the sequence of the reference nucleotide sequence. Nucleotides are referred to herein by the standard one-letter designation (A, C, G, or T). Due to the degeneracy of the genetic code, a “nucleotide sequence variant” can either result in a change in the respective reference amino acid sequence, i.e. in an “amino acid sequence variant” or not. Preferred sequence variants are such nucleotide sequence variants, which do not result in amino acid sequence variants (silent mutations), but other non-silent mutations are within the scope as well, in particular mutant nucleotide sequences, which result in an amino acid sequence, which is at least 80%, preferably at least 90%, more preferably at least 95% sequence identical to the reference sequence.

An “amino acid sequence variant” has an altered sequence in which one or more of the amino acids in the reference sequence is deleted or substituted, or one or more amino acids are inserted into the sequence of the reference amino acid sequence. As a result of the alterations, the amino acid sequence variant has an amino acid sequence which is at least 80% identical to the reference sequence, preferably, at least 90% identical, more preferably at least 95% identical, most preferably at least 99% identical to the reference sequence. Variant sequences which are at least 90% identical have no more than 10 alterations, i.e. any combination of deletions, insertions or substitutions, per 100 amino acids of the reference sequence. Percent identity is determined by comparing the amino acid sequence of the variant with the reference sequence using computer programs well-known in the art, in particular according to the MEGALIGN project in the DNA STAR program.

While it is possible to have non-conservative amino acid substitutions, it is preferred that the substitutions be conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g. alanine, valine, leucine and isoleucine, with another; substitution of one hydoxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, methionine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

Importantly, the alterations in the sequence variants do not abolish the functionality of the respective reference sequence, in the present case e.g. the functionality of mutant immunogenic components to trigger an immune response of sufficient strength. Guidance in determining which nucleotides and amino acid residues, respectively, may be substituted, inserted or deleted without abolishing such functionality are found by using computer programs well known in the art, for example, DNASTAR software.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

The inventors of the present invention have surprisingly found that the use of a pentameric soluble protein complex vaccine obtainable by the inventive vector, which encodes the HCMV glycoproteins gH, gL, pUL128, pUL130 and pUL131 results in the formation of high numbers of predominantly neutralizing antibodies against HCMV infection of fibroblasts, epithelial, endothelial, and myeloid cells. Throughout the present invention, the protein and gene encoding for HCMV glycoprotein UL128, UL130, or UL131A may be referred to as pUL128, pUL130, pUL131, or UL131, respectively. Likewise, throughout the present invention the HCMV pentameric complex formed by the surface glycoproteins gH, gL, pUL128, pUL130 and pUL131A may e.g. also referred to as gHgLpUL128L, or HCMV pentameric complex, or HCMV pentamer, or pentamer.

Thus, according to a first aspect the present invention provides for a vector for expressing HCMV glycoproteins in a mammalian cell and wherein the vector comprises a transcription system. This transcription system comprises in general
(i) at least one promoter operable in a mammalian cell and operably linked to
(ii) at least one open reading frame (ORF) comprising at least one nucleotide sequence selected from the group consisting of nucleotide sequences encoding the HCMV glycoproteins gH, gL, pUL128, pUL130 and pUL131 or sequence variants thereof, i.e. an amino acid sequence according to SEQ ID NO:21, SEQ ID NO:25, SEQ ID NO:3, SEQ ID NO:7 and SEQ ID NO:11 or sequence variants thereof,
   whereby the vector comprises each of the nucleotide sequences selected from the group consisting of nucleotide sequences encoding the HCMV glycoproteins gH, gL, pUL128, pUL130 and pUL131 or sequence variants thereof, i.e. an amino acid sequence according to SEQ ID NO:21, SEQ ID NO:25, SEQ ID NO:3, SEQ ID NO:7 and SEQ ID NO:11 or the sequence variants thereof.

In the inventive gene expression system, the preferred nucleotide sequences encoding gH and gL are according to SEQ ID NO:22, SEQ ID NO:26 or sequence variants thereof and the preferred nucleotide sequences encoding pUL128, pUL130 and pUL131 are according to SEQ ID NO:4, SEQ ID NO:8, SEQ ID NO:12 or sequence variants thereof, respectively.

For example, the inventive vector preferably comprises at least two transcription units, each of which comprises an ORF, operably linked to a promoter. Each of the ORFs may further comprise e.g. a 5′ start codon and encodes two or more HCMV viral glycoproteins, such as e.g. gH (e.g. by SEQ ID NO:22), gL (e.g. by SEQ ID NO:26), pUL128 (e.g. by SEQ ID NO:4), pUL130 (e.g. by SEQ ID NO:8), or pUL131 (e.g. by SEQ ID NO:12), or sequence variants thereof. Even more preferably, the vector of the inventive gene expression system comprises operably linked (i) a first promoter operable in a mammalian cell, (ii) a first open reading frame (ORF), which comprises a 5′ start codon, and a nucleotide sequence, which comprises SEQ ID NO:22 and SEQ ID NO:26 or sequence variants thereof, (iii) a second promoter operable in said mammalian cell and (iv) a second open reading frame (ORF), which comprises a 5′ start codon and a nucleotide sequence according to SEQ ID NO:4, SEQ ID NO:8 and SEQ ID NO:12 or sequence variants thereof.

The ORFs may e.g. further comprise nucleotide sequences which encode one or more of the self-processing peptides of the Foot-and-Mouth Disease virus, such as e.g. P2A (e.g. SEQ ID NO:24), T2A (e.g. SEQ ID NO:6), or F2A (e.g. SEQ ID NO:10), which will result in ribosomal skipping, which impairs normal peptide bond formation upon translation and results in the generation of two or more proteins from one mature mRNA (cf. for example Palmenberg, A. C. et al. Virology 190, 754-762 (1992)). The 2A peptide consensus motif, which is typically associated with cleavage activity is Asp-Val/Ile-Glu-X-Asn-Pro-Gly-(P2B-Pro) (SEQ ID NO:56) and will result in cleavage between the P2A glycine and the 2B proline. Other peptide sequences that result in ribosomal skipping may be also be used in the present invention for the generation of two or more, e.g. two or three, HCMV glycoproteins from one mature mRNA, such as e.g. T2A (e.g. SEQ ID NO:5), or F2A (e.g. SEQ ID NO:9).

Preferably, within each open reading frame of the vector according to the present invention the nucleotide sequences selected from the group consisting of nucleotide sequences encoding gH, gL, UL128, UL130 and UL131 or sequence variants thereof are separated from each other by a nucleotide sequence encoding a ribosomal skipping site, preferably by a nucleotide sequence encoding the amino acid sequence according to SEQ ID NO: 56.

It is also preferred that in the vector according to the present invention a first and a second open reading frame each comprise at least one nucleotide sequence encoding an amino acid selected from the group consisting of SEQ ID NO:23, SEQ ID NO:27, SEQ ID NO:29 and sequence variants thereof.

More preferably, the vector according to the present invention comprises a transcription system comprising:

(i) a first promoter operable in a mammalian cell and operably linked to
(ii) a first open reading frame comprising a nucleotide sequence encoding gH and a nucleotide sequence encoding gL, or sequence variants thereof; and
(iii) a second promoter operable in a mammalian cell and operably linked to
(iv) a second open reading frame comprising a nucleotide sequence encoding UL128, a nucleotide sequence encoding UL130 and a nucleotide sequence encoding UL131, or sequence variants thereof;
   wherein:
(a) the first open reading frame further comprises a nucleotide sequence encoding a ribosomal skipping site having an amino acid sequence selected from the group consisting of SEQ ID NO:23, SEQ ID NO:27, SEQ ID NO:29 and sequence variants thereof; and
(b) the second open reading frame further comprises at least one nucleotide sequence encoding a ribosomal skipping site having an amino acid sequence selected from the group consisting of SEQ ID NO:5, SEQ ID NO:9, SEQ ID NO:23, SEQ ID NO:27, SEQ ID NO:29 and sequence variants thereof, preferably from the group consisting of SEQ ID NO:5, SEQ ID NO:9, and sequence variants thereof.

Thereby, it is even more preferred that in the first open reading frame the nucleotide sequence encoding a ribosomal skipping site having an amino acid sequence selected from the group consisting of SEQ ID NO:23, SEQ ID NO:27, SEQ ID NO:29 and sequence variants thereof is arranged between a nucleotide sequence encoding gH and a nucleotide sequence encoding gL or sequence variants thereof; and wherein in the second open reading frame a nucleotide sequence encoding a first ribosomal skipping site having an amino acid sequence selected from the group consisting of SEQ ID NO:5, SEQ ID NO:9, SEQ ID NO:23, SEQ ID NO:27, SEQ ID NO:29 and sequence variants thereof, preferably from the group consisting of SEQ ID NO:5, SEQ ID NO:9, and sequence variants thereof, is arranged between a nucleotide sequence encoding UL128 and a nucleotide sequence encoding UL130 or sequence variants thereof and a nucleotide sequence encoding a second ribosomal skipping site having an amino acid sequence selected from the group consisting of SEQ ID NO:5, SEQ ID NO:9, SEQ ID NO:23, SEQ ID NO:27, SEQ ID NO:29 and sequence variants thereof, preferably from the group consisting of SEQ ID NO:5, SEQ ID NO:9, and sequence variants thereof, is arranged between a nucleotide sequence encoding UL130 and a nucleotide sequence encoding UL131 or sequence variants thereof.

The term “vector” as used herein, in particular with the inventive gene expression system, refers to a nucleic acid, into which fragments of nucleic acid may be inserted or cloned and which is typically a plasmid, a viral vector, a cosmid or an artificial chromosome, whereby a plasmid is preferred. Preferably, the vector is an expression vector, which is optimized for the expression of a peptide or a protein, whereby an expression vector suitable for a mammalian expression system is particularly preferred. Accordingly, it is particularly preferred that a sequence used in the vector, most preferably all sequences used in the vector, are codon optimized for expression in mammalian cells. Importantly, the term “vector” as used herein refers to a single entity, e.g. one plasmid is one vector, whereas five plasmids are five vectors. Preferably, the vector is a DNA construct.

Since the inventive vector is usually used for the preparation of a vaccine for use in mammals, in particular in humans, the vector is in general designed for this use. To this end the vector is preferably suitable for expressing HCMV glycoproteins in a mammalian cell and used in this context, since vaccine preparations are advantageously based on a mammalian expression system for safety aspects including e.g. the provision of an appropriate glycosylation pattern.

Accordingly, the vector according to the present invention as well as the respective gene expression system is preferably not based on a viral replicon system, on a bacterial artificial chromosome (BAC)/Modified Vaccinia Ankara (MVA) system, or on a baculovirus system.

Thus, it is preferred that the vector according to the present invention:
(a) is not a self-replicating RNA molecule nor does it comprise a self-replicating RNA molecule;
(b) is not an alphavirus replicon nor does it comprise an alphavirus replicon; and/or
(c) does not comprise any sequence encoding an alphavirus non-structural protein such as NSP1, NSP2, NSP3 and NSP4.

Thereby, option (a) is preferred, i.e. it is preferred that the vector according to the present invention is not a self-replicating RNA molecule nor does it comprise a self-replicating RNA molecule.

It is also preferred that the vector according to the present invention:
(a) is not packaged into viral replicon particles;
(b) is not encapsulated in lipid nanoparticles; and
(c) is not formulated with CMF34.
   CMF34 is a cationic emulsion including 4.3% w/v squalene, 0.5% Tween 80, 0.5% SPAN85, and 4.4 mg/mL DOTAP.

Moreover, it is also preferred that the vector according to the present invention:
(a) is not derived from and not comprised by a bacterial artificial chromosome (BAC) construct; and/or
(b) is not an MVA-derived vector.

In particular, the vector according to the present invention is preferably not a bacterial artificial chromosome (BAC) construct. A BAC is a DNA construct, which is based on a functional fertility plasmid (or F-plasmid), and which is typically used for transforming and cloning in bacteria. Thus, a BAC typically serves as a cloning vector. MVA (Modified Vaccinia virus Ankara) is a replication-deficient attenuated poxvirus. Recombinant MVA-based vectors were developed, for example for vaccination, e.g. G. Di Lullo, et al. (2009): Marker gene swapping facilitates recombinant Modified Vaccinia Virus Ankara production by host-range selection. In: Journal of virological methods. Vol. 156, p. 37-43. More preferably, the vector according to the present invention is not derived from a poxvirus.

Furthermore, it is also preferred that the vector according to the present invention:
(a) does not comprise a sequence encoding a viral capsid or capsid precursor protein; and/or
(b) the vector backbone of said vector is neither pRBT136 nor pRBT393.

The vector backbones pRBT136 and pRBT393 relate to a baculovirus system and are described, for example, in WO 2014/068001 A1. Namely, pRBT136 is suitable for recombinant protein expression using the baculovirus expression system (BEVS) and contains two promoters P1 and P2 (p10, polh) and two terminator sequences T1 and T2, which are SV40 and HSVtk. For propagation in yeast the pRBT136 vector contains an origin of replication, e.g. 2 micron, and a marker gene, e.g. URA3. Furthermore the vector contains the transposon sites left and right for transposition of the transgenes from the transfer vector into bacmids, a loxP site for site specific homologous recombination (plasmid fusion), origins of replication, ampicillin, chloramphenicol and gentamycin resistance genes, and defined restriction sites. For the expression in mammalian cells, either by transduction with a baculovirus or transient expression, the vector backbone pRBT 393 contains in addition a promoter selected from pCMV, ie1 and lef2, and a terminator selected from SV40 pA, BHGpA and HSVtk.

Preferably, the vector according to the present invention is not derived from a retroviral vector, a lentiviral vector, an adenoviral vector, or an adeno-associated viral vector. More preferably, the vector according to the present invention is not derived from a viral vector.

Even more preferably, the vector according to the present invention is not a retroviral vector, a lentiviral vector, an adenoviral vector, or an adeno-associated viral vector. Particularly preferably, the vector according to the present invention is not a viral vector.

In particular, the term “derived from” (e.g. a viral vector) refers to any vector, wherein at least 50%, preferably at least 70%, more preferably at least 80%, even more preferably at least 90% and particularly preferably at least 95% of the backbone sequence of the vector is of viral origin. Typically, the backbone of a vector refers to the vector without the open reading frames, preferably the backbone of a vector refers to the vector without those open reading frames which comprise a nucleotide sequence encoding gL, a nucleotide sequence encoding gH, a nucleotide sequence encoding UL128, a nucleotide sequence encoding UL130, and/or a nucleotide sequence encoding UL128.

Preferably, the vector according to the present invention is a plasmid vector, more preferably a DNA plasmid vector, which is suitable for expression in mammalian cells, preferably in mammalian cell lines. If necessary—in particular if the vector according to the present invention comprises more than one ORFs, e.g. two, three, four or five ORFs, preferably two ORFs—virtually any vector (e.g. any commercially available vector) for expression of a single protein of interest in mammalian cells can be transformed into a vector expressing more than one proteins of interest by inserting one or more additional promoter(s), whereby in the vector according to the present invention the number of ORFs preferably corresponds to the number of promoters, in particular every promoter of the vector according to the present invention is preferably operably linked to an ORF. For example, a commercially available mammalian expression vector may be used, wherein a first ORF may be inserted at the site in the vector provided for this purpose, e.g. at the multiple cloning site (MCS), and a complete cassette encoding an additional promoter, which is preferably identical to the other promoter(s) of the vector, followed by and operably linked to a second ORF, may be inserted directly downstream of the first cassette. Further additional cassettes encoding additional promoters and ORFs may also be inserted, e.g. by the same principle.

More preferably, the vector is a “double gene mammalian expression vector” (also referred to as “two gene mammalian expression vector”), i.e. a vector, which is designed for simultaneous expression of two genes in mammalian cells, e.g. in mammalian cell lines. Such vectors and/or appropriate construction methods are commercially available. Preferably, a double gene vector may be constructed by using the Lonza expression vector system, e.g. by cloning the first ORF into a Lonza primary expression vector, e.g. Lonza pEE 12.4 or Lonza pEE 14.4, and cloning the second ORF into a Lonza accessory expression vector, e.g. Lonza pEE 6.4, and constructing a double gene mammalian expression vector on the basis of these two vectors for example by using the Lonza G S System™ (cf. WO 2008/148519 A2 and Zettlitz, K. A. in “Antibody Engineering, Vol. 1”; Kontermann R. and Dubel S. (eds); Springer Heidelberg 2010, 2^{nd} edition; chapter 20). Other preferred examples of double gene mammalian expression vectors include pBudCE4.1 vectors (Life Technologies), pBI vectors (Clontech; e.g. pBI-CMV1), pVitro vectors (Invivogen), and pBICEP™ vectors (Sigma-Aldrich).

Such a double gene mammalian expression vector is particularly preferred in the context of a vector according to the present invention comprising two promoters each of them operably linked to an open reading frame, wherein the first open reading frame comprises 1 to 4 of the nucleotide sequences encoding gH, gL, UL128, UL130 and UL131 or sequence variants thereof and the second open reading frame comprises the nucleotide sequences encoding those of gH, gL, UL128, UL130 and UL131 or sequence variants thereof, which are not comprised by the first open reading frame. In particular, such a double gene mammalian expression vector is particularly preferred in the context of a vector according to the present invention, wherein the vector comprises a transcription system comprising
(i) a first promoter operable in a mammalian cell and operably linked to
(ii) a first open reading frame comprising a nucleotide sequence encoding gH and a nucleotide sequence encoding gL or sequence variants thereof; and
(iii) a second promoter operable in a mammalian cell and operably linked to
(iv) a second open reading frame comprising a nucleotide sequence encoding UL128, a nucleotide sequence encoding UL130 and a nucleotide sequence encoding UL131 or sequence variants thereof.

Preferably, the vector according to the present invention, which is suitable for expression in mammalian cells, e.g. a plasmid vector for expression in mammalian cells, is suitable for stable transfection, i.e. for integration into the genome of the host cells. The examples of preferred vectors described above, e.g. vectors provided by Lonza Biologics in the context of the LONZA GS Gene Expression System′, e.g. the Lonza pEE vectors, pBudCE4.1 vectors (Life Technologies), pBI vectors (Clontech; e.g. pBI-CMV1), pVitro vectors (Invivogen), or pBICEP™ vectors (Sigma-Aldrich), can be used for stable transfection.

Thereby, the vectors provided by Lonza Biologics in the context of the LONZA GS Gene Expression System™ are particularly preferred since the LONZA GS Gene Expression System™ is based on glutamine synthetase (GS) as selection marker. Accordingly, the respective vectors provided by Lonza include a nucleotide sequence encoding GS, but the respective promoter is a weak promoter. This allows for selection of such clones of stably transfected cells, wherein the integration in the host cell genome occurred at loci of high level of transcription. The principle of the LONZA GS Gene Expression System™ is described in WO 87/04462 A1.

In particular, the vector may contain one or more unique restriction sites for this purpose, and may be capable of autonomous replication in a defined host or organism such that the cloned sequence is reproduced. The vector molecule may confer some well-defined phenotype on the host organism which is either selectable or readily detected. Some components of a vector may be a DNA molecule further incorporating a DNA sequence encoding regulatory elements for transcription, translation, RNA stability and replication, or e.g. antibiotic selection.

The vector may e.g. also comprise nucleotide sequences which encode peptide or protein moieties which will facilitate the purification of encoded inventive protein products, such as a tag sequence, e.g. a His-tag or a Strep-tag sequence, for example a 6×His-tag (e.g. SEQ ID NO:42), or e.g. a Strep-Tag® (e.g. SEQ ID NO:18 or SEQ ID NO:40), which may for example be coupled to a cleavage site, e.g. a TEV cleavage site (e.g. SEQ ID NO:14). This enables a removal of the tag, which e.g. facilitates the purification, after purification. Thus, the vaccine does not contain this tag anymore, thereby ensuring an antibody response of high specificity.

Preferably, in the vector according to the present invention a nucleotide sequence encoding a tag sequence does not occur in association with a nucleotide sequence encoding gH or sequence variants thereof, and/or a nucleotide sequence encoding a tag sequence does not occur in association with a nucleotide sequence encoding gL or sequence variants thereof. Thereby, a nucleotide sequence encoding a tag sequence “occurring in association with” a nucleotide sequence encoding gH or gL means that upon expression subunit gH is not linked to a tag sequence and/or subunit gL is not linked to a tag sequence.

Thereby, it is ensured that if a tag sequence is present, e.g. to facilitate the purification of encoded inventive protein products, such a tag sequence is not present at gH or gL. Thereby, an excess of gH/gL dimer and/or the formation of multimers containing e.g. more than one gH and/or gL subunit is avoided. Thus, the 1:1:1:1:1 stoichiometry of the pentamer is further supported.

For example, a preferred vector according to the present invention comprises—as described above—a transcription system comprising
(i) a first promoter operable in a mammalian cell and operably linked to
(ii) a first open reading frame comprising a nucleotide sequence encoding gH and a nucleotide sequence encoding gL or sequence variants thereof; and
(iii) a second promoter operable in a mammalian cell and operably linked to
(iv) a second open reading frame comprising a nucleotide sequence encoding UL128, a nucleotide sequence encoding UL130 and a nucleotide sequence encoding UL131 or sequence variants thereof.

Thereby, it is preferred that the first open reading frame, which comprises a nucleotide sequence encoding gH and a nucleotide sequence encoding gL or sequence variants thereof, does not comprise a nucleotide sequence encoding a tag sequence. In other words, no nucleotide sequence encoding a tag sequence is present in the first ORF. Thereby, upon expression neither gH nor gL are linked to a tag sequence. The second ORF, which comprises a nucleotide sequence encoding UL128, a nucleotide sequence encoding UL130 and a nucleotide sequence encoding UL131 or sequence variants thereof, may or may not comprise a tag sequence.

Moreover, the vector according to the present invention is preferably constructed such that upon expression a tag sequence is preferably present at the C-terminus of UL131, more preferably upon expression a tag sequence is only present at the C-terminus of UL131, i.e. no tag sequence is present at the N- or C-terminus of gH, gL, UL128 and UL130. Thereby, superior purification results can be achieved. Accordingly, the vector according to the present invention preferably comprises a nucleotide sequence encoding a tag sequence, in particular a His-Tag and/or a Strep-Tag sequence, which is located no more than 100 nucleotides downstream of the 3′-end of a nucleotide sequence encoding UL131. Preferably, the nucleotide sequence encoding a tag sequence, in particular a His-Tag and/or a Strep-Tag sequence, is located no more than 70 nucleotides downstream of the 3′-end of a nucleotide sequence encoding UL131, more preferably the nucleotide sequence encoding a tag sequence, in particular a His-Tag and/or a Strep-Tag sequence, is located no more than 50 nucleotides downstream of the 3′-end of a nucleotide sequence encoding UL131, even more preferably the nucleotide sequence encoding a tag sequence, in particular a His-Tag and/or a Strep-Tag sequence, is located no more than 30 nucleotides downstream of the 3′-end of a nucleotide sequence encoding UL131 and particularly preferably the nucleotide sequence encoding a tag sequence, in particular a His-Tag and/or a Strep-Tag sequence, is located no more than 20 nucleotides downstream of the 3′-end of a nucleotide sequence encoding UL131.

The nucleotide sequence encoding the tag sequence may be located, for example, directly downstream of the 3′-end of a nucleotide sequence encoding UL131 (i.e. without any nucleotides located in between the nucleotide sequence encoding the tag sequence and the 3′-end of a nucleotide sequence encoding UL131) or the tag sequence may be, for example, separated from nucleotide sequence encoding UL131 by one or more other nucleotide sequences, preferably by a nucleotide sequence encoding a linker and/or a nucleotide sequence encoding a peptide cleavage site. Preferably, the nucleotide sequence encoding the tag sequence is separated from nucleotide sequence encoding UL131 by a nucleotide sequence encoding a linker and/or a nucleotide sequence encoding a peptide cleavage site.

More preferably, the vector according to the present invention does not comprise a nucleotide sequence encoding a tag sequence, in particular a His-Tag or a Strep-Tag sequence, which is located adjacently to the 3′-end of a nucleotide sequence encoding gH and/or gL, even more preferably the vector according to the present invention does not comprise a nucleotide sequence encoding a tag sequence, in particular a His-Tag or a Strep-Tag sequence, which is located adjacently to the 3′-end of a nucleotide sequence encoding gH, gL, UL128 and/or UL130. Thereby, “located adjacently” means that the tag sequence occurs in association with a nucleotide sequence encoding a subunit as described herein, i.e. upon expression the tag sequence is linked to the respective subunit. In particular, the meaning of the term “located adjacently” includes an (optional) separation, for example by up to 1000, up to 500, up to 200, up to 100 nucleotides, e.g. by a nucleotide sequence encoding a linker and/or a nucleotide sequence encoding a peptide cleavage site. However, it is understood that a nucleotide sequence encoding another subunit of the HCMV pentamer located in between the nucleotide sequence encoding the tag sequence and the nucleotide sequence encoding the HCMV pentamer subunit in question is not encompassed by the meaning of the term “located adjacently”.

Preferably, in the present invention the tag sequence comprises or consists of an amino acid sequence selected from the group consisting of SEQ ID NOs: 17, 39, 41 and sequence variants thereof, the peptide cleavage site comprises or consists of an amino acid sequence according to SEQ ID NO: 13 or sequence variants thereof, and the linker sequence comprises or consists of an amino acid sequence according to SEQ ID NO: 15 or sequence variants thereof.

More preferably, the vector according to the present invention comprises a nucleotide sequence encoding the tag sequence, which comprises or consists of an nucleotide sequence selected from the group consisting of SEQ ID NOs: 18, 40, 42 and sequence variants thereof, a nucleotide sequence encoding the peptide cleavage site, which comprises or consists of a nucleotide sequence according to SEQ ID NO: 14 or sequence variants thereof, and a nucleotide sequence encoding the linker sequence, which comprises or consists of a nucleotide sequence according to SEQ ID NO: 16 or sequence variants thereof.

For example, it is preferred that in the vector according to the present invention a nucleotide sequence encoding a tag sequence, e.g. according to any of SEQ ID NOs: 17, 39, or 41 or sequence variants thereof, is located no more than 100, preferably no more than 70, more preferably no more than 50, even more preferably no more than 30, particularly preferably no more than 20 nucleotides downstream of the 3′-end of a nucleotide sequence encoding UL131, e.g. according to any of SEQ ID NOs: 11 or 33 or sequence variants thereof, whereby the nucleotide sequence encoding the tag sequence is separated from nucleotide sequence encoding UL131 by a nucleotide sequence encoding a linker, e.g. according to SEQ ID NO: 15 or sequence variants thereof, and/or by a nucleotide sequence encoding a peptide cleavage site, e.g. according to SEQ ID NO: 13 or sequence variants thereof.

Accordingly, the vector may further comprise e.g. spacer sequences between the individual tags, such as e.g. a GS linker according to SEQ ID NO:16. The sequences may e.g. be comprised on the vector singly, or preferably in combination, such as e.g. in 5′-3′ direction SEQ ID NO:14, SEQ ID NO:42, SEQ ID NO:16 and SEQ ID NO:18 or sequence variants thereof, or e.g. SEQ ID NO:14, SEQ ID NO:42, SEQ ID NO:42, SEQ ID NO:16 and SEQ ID NO:40 or sequence variants thereof, or e.g. SEQ ID NO:14, SEQ ID NO:16, SEQ ID NO:42, and SEQ ID NO:18 or sequence variants thereof, or e.g. SEQ ID NO:14, SEQ ID NO:16, SEQ ID NO:42, and SEQ ID NO:40 or sequence variants thereof, or e.g. SEQ ID NO:14, SEQ ID NO:16, SEQ ID NO:42, SEQ ID NO:42 and SEQ ID NO:18 or sequence variants thereof, or e.g. SEQ ID NO:14, SEQ ID NO:16, SEQ ID NO:42, SEQ ID NO:42 and SEQ ID NO:40 or sequence variants thereof, or e.g. SEQ ID NO:16, SEQ ID NO:14, SEQ ID NO:42, SEQ ID NO:42 and SEQ ID NO:18 or sequence variants thereof, or e.g. SEQ ID NO:16, SEQ ID NO:14, SEQ ID NO:42, SEQ ID NO:42 and SEQ ID NO:40 or sequence variants thereof, or e.g. SEQ ID NO:14, SEQ ID NO: SEQ ID NO:42, SEQ ID NO:16 and SEQ ID NO:40 or sequence variants thereof, or e.g. SEQ ID NO:16, SEQ ID NO:14 SEQ ID NO:18, and SEQ ID NO:42 or sequence variants thereof, or e.g. SEQ ID NO:14 and SEQ ID NO:42 or sequence variants thereof, or e.g. SEQ ID NO:16, SEQ ID NO:14 and SEQ ID NO:42 or sequence variants thereof, or e.g. SEQ ID NO:14, SEQ ID NO:16 and SEQ ID NO:42 or sequence variants thereof, or e.g. SEQ ID NO:14 and SEQ ID NO:18 or sequence variants thereof, or e.g. SEQ ID NO:14 and SEQ ID NO:40 or sequence variants thereof. The sequences as disclosed above may e.g. be comprised on the 5′ end, or e.g. 3′ end of each of the ORFs of the inventive transcription system as part of the inventive vector, preferably, the sequences as disclosed above are 3′ or at the 3′ end of at least one of the ORFs of the inventive transcriptions system, e.g. the sequences may be present at the 3′ end of a first ORF of the inventive transcription system, or e.g. at the 3′ end of a second ORF, or e.g. may be present at the 3′ ends of a first and second ORF of the inventive vector.

Alternatively, it is also preferred that the vector according to the present invention does not comprise a nucleotide sequence encoding a tag sequence, e.g. a His-tag or a Strep-tag. Thereby, it is even more preferred if such a vector according to the present invention, which does not comprise a nucleotide sequence encoding a tag sequence, does also not comprise a nucleotide sequence encoding a cleavage site.

For example, the vector may also comprise sequences, which facilitate the secretion of the proteins encoded by the nucleotide sequences as disclosed in the present invention, e.g. the vector of the inventive gene expression system may comprise signal peptides. The term “signal peptide” (sometimes referred to as signal sequence, leader sequence or leader peptide) as used in the present invention refers to a peptide of typically 5-30 amino acids in length present at the N-terminus of the majority of newly synthesized proteins that are destined towards the secretory pathway. Signal peptides may be artificial, or may be derived from immunoglobulins, such as e.g. the murine IgG signal peptide (e.g. as encoded by the nucleotide sequence according to SEQ ID NO:20), or e.g. viral signal peptides, such as e.g. encoded by SEQ ID NO:2. For example, a first and/or a second ORF of the inventive vector may comprise as a 5′ sequence a signal peptide sequence as defined above, or e.g. any one of the HCMV surface glycoproteins as disclosed herein and as encoded in a first and/or second ORF may e.g. comprise a signal sequence, e.g. SEQ ID NO:20, or SEQ ID NO:2, or sequence variants thereof, on their respective 5′ ends, or e.g. if referred to in terms of amino acid sequence, the HCMV surface glycoproteins as disclosed in the present invention may comprise at their N-terminus a signal peptide according to SEQ ID NO:1, or SEQ ID NO:19, or sequence variants thereof. For example, the sequence encoding the gH signal peptide may preferably be replaced by a sequence encoding the IgG leader sequence, e.g. SEQ ID NO:2 or sequence variants thereof.

The term “promoter” as used herein refers to a nucleotide sequence, preferably a DNA sequence, that determines the site of transcription initiation of RNA polymerase, e.g. a promoter may be a regulatory sequence within about 200 base pairs of the transcription start site of RNA polymerase II (RNAP II), but may also comprise DNA sequence elements within −1000 bp to about −100 bp of the transcription start site of RNAP II. Accordingly, the first promoter of the inventive gene expression system may be e.g. a murine CMV promoter (MCMV), a human CMV (HCMV), e.g. a HCMV-MIE (major immediate early) promoter, a SV40, a HSV-TK, an EF1-1 or PGK promoter. The use of murine CMV promoter for expressing recombinant proteins in CHO cells has been described in prior art, such as e.g. in WO 2004/009823, whereby the respective parts of this document are incorporated by reference herein. Thus, a first promoter of the inventive vector is preferably one of a MCMV, a HCMV, a SV40, a HSV-TK, an EF1-1α or PGK promoter. For example, a first promoter may be e.g. a MCMV promoter, or e.g. a HCMV promoter, or e.g. a SV40 promoter, or e.g. a HSV-TK promoter, or e.g. an EF1-1a promoter or e.g. a PGK promoter as defined above. For example, the at least one ORF of the inventive vector may preferably further comprise a first promoter and operably linked in 5′-3′ direction nucleotide sequences encoding gH and gL, e.g. nucleotide sequences according to SEQ ID NO:22 and SEQ ID NO:26 or sequence variants thereof.

Moreover, the promoter of the inventive vector may also be e.g. an inducible promoter, such as the tetracycline-inducible promoter (Gossen and Bujard, (1992) PNAS Jun. 15; 89(12):5547-51), or an IPTG-inducible system (e.g. such as that disclosed by Grespy et al. PLoS One. 2011 Mar. 21; 6(3):e18051), which allow for a temporal control of gene expression of the genes operably linked to the first promoter of the inventive gene expression system.

In addition, the at least one ORF of the inventive vector may preferably further comprise a 5′ start codon, e.g. the triplet ATG, which encodes the amino acid methionine (Met). The start codon of the at least one ORF of the inventive gene expression system may e.g. also be comprised in a Kozak sequence, e.g. the 5′ start codon may be comprised in the sequence 5′-GCCACCATG or the start codon may be downstream of the Kozak sequence, which results in an improved translation efficacy of the matured RNAP II transcript.

The vector may preferably further comprise a second promoter as defined above, e.g. a promoter identical or different to a first promoter of the inventive gene expression system, such as e.g. murine CMV promoter (MCMV), a human CMV (HCMV), e.g. a HCMV-MIE (major immediate early) promoter, a SV40, a HSV-TK, an EF1-1 or PGK promoter. Accordingly, the vector of the inventive gene expression system may comprise e.g. as first and second promoter (MCMV) and as second promoter a human CMV, or e.g. as first promoter a SV40 and as second promoter a HSV-TK, or e.g. as first promoter an EF1-1 promoter and as second promoter a PGK promoter, or e.g. as first and second promoter an MCMV promoter, or e.g. as first and second promoter an HCMV promoter, e.g. a HCMV-MIE (major immediate early) promoter, or e.g. a SV40 promoter as first promoter and a MCMV promoter as second promoter, or e.g. a HCMV promoter as first promoter and a SV40 promoter as second promoter, or e.g. an inducible promoter, such as e.g. as first and second promoter, or e.g. an EF-1 promoter as first and second promoter, or e.g. an EF-1 promoter as first promoter and a PGK promoter as second promoter.

Preferably, if the vector according to the present invention comprises more than one ORF, the promoters, which are operably linked to each of the ORFs comprised by the vector, allow for a similar strength of expression, i.e. upon expression the ORFs yield products in similar quantities. Since the exemplary promoters mentioned above are all strong promoters in mammalian cells, they may be used in combination. More preferably, if the vector according to the present invention comprises more than one ORF, the promoters, which are operably linked to each of the ORFs comprised by the vector, are identical. Even more preferably, the vector according to the present invention comprises a first promoter operable in a mammalian cell and operably linked to a first open reading frame and a second promoter operable in a mammalian cell and operably linked to a second open reading frame, wherein the first and the second promoter are identical. Thereby, it is preferred that the first and the second promoter are CMV promoters, e.g. MCMV or HCMV promoters, preferably MCMV promoters or HCMV-MIE promoters. Thereby, it is also preferred that the first open reading frame (to which the first promoter is operably linked) comprises a nucleotide sequence encoding gH and a nucleotide sequence encoding gL or sequence variants thereof and the second open reading frame (to which the second promoter, which is identical to the first promoter, is operably linked) comprises a nucleotide sequence encoding UL128, a nucleotide sequence encoding UL130 and a nucleotide sequence encoding UL131 or sequence variants thereof. Such a vector design with identical promoters further supports the equimolar expression of the subunits gH, gL, UL128, UL130 and UL131 of the HCMV pentameric glycoprotein complex, i.e. in a 1:1:1:1:1 stoichiometry of the subunits gH, gL, UL128, UL130 and UL131. Moreover, since the two ORFs are located on a single vector, the two ORFs are typically integrated into the same genomic site. Thus, the two identical promoters are located in a site with a similar transcriptional activity. If the two ORFs would be inserted into different sites, in contrast, the different level of chromatin accessibility for transcription likely impairs a balanced expression of the two ORFs.

The term “identical” as used herein means that each “identical” promoter is of the same type, for example each identical promoter is a hCMV-MIE promoter or each identical promoter is a MCMV promoter or each identical promoter is any other specified promoter of the same type. More preferably, each “identical” promoter has the same nucleotide sequence. In particular, the term “identical” as used herein does imply any number of promoters contained in the vector. That means in particular that the term “identical” as used herein does not necessarily imply that only one single promoter exists in a vector according to the present invention wherein all promoters are identical. Instead, a vector according to the present invention, wherein (all) promoters are identical, may have one or more promoters of the same type as described above. For example, a vector having a first promoter and a second promoter, wherein the first and the second promoter are identical, has preferably (at least) two promoters of the same type, preferably of the same sequence as described above.

Thus, a second promoter of the inventive vector is preferably one of a MCMV, a HCMV, e.g. a HCMV-MIE (major immediate early) promoter, a SV40, a HSV-TK, an EF1-1a or PGK promoter. For example, a second promoter may be e.g. a MCMV promoter, or e.g. a HCMV promoter, e.g. a HCMV-MIE (major immediate early) promoter, or e.g. a SV40 promoter, or e.g. a HSV-TK promoter, or e.g. an EF1-1a promoter or e.g. a PGK promoter as defined above.

Accordingly, the inventive vector further comprises a second ORF, which comprises a 5′ start codon as defined above and the nucleotide sequence encoding SEQ ID NO:4, SEQ ID NO:8 and SEQ ID NO:12 or sequence variants thereof. Accordingly, the second ORF of the inventive gene expression system comprises a 5′ start codon, e.g. a 5′ start codon. The start codon may be comprised by the Kozak sequence as defined above or may be downstream of the Kozak sequence and a nucleic sequence encoding SEQ ID NO:4, SEQ ID NO:8 and SEQ ID NO:12 or sequence variants thereof, or e.g. SEQ ID NO:8, SEQ ID NO:4 and SEQ ID NO:12 or sequence variants thereof, or e.g. SEQ ID NO:12, SEQ ID NO:8 and SEQ ID NO:4 or sequence variants thereof, or e.g. SEQ ID NO:12, SEQ ID NO:4 and SEQ ID NO:8 or sequence variants thereof.

Moreover, a second ORF of the inventive vector may comprise at least a 5′ start codon and a nucleotide sequence encoding SEQ ID NO:4, SEQ ID NO:8 or sequence variants thereof and SEQ ID NO:12, SEQ ID NO:14, SEQ ID NO:16, SEQ ID NO:18 and SEQ ID NO:42 or sequence variants thereof. Accordingly, a second ORF of the inventive vector may comprise a start codon as defined above, and a nucleotide sequence encoding SEQ ID NO:4, SEQ ID NO:8 and SEQ ID NO:12, SEQ ID NO:14, SEQ ID NO:16, SEQ ID NO:18 and SEQ ID NO:42 or sequence variants thereof, or e.g. SEQ ID NO:8, SEQ ID NO:4 and SEQ ID NO:12, SEQ ID NO:14, SEQ ID NO:16, SEQ ID NO:18 and SEQ ID NO:42 or sequence variants thereof, or e.g. SEQ ID NO:12, SEQ ID NO:8 and SEQ ID NO:4, SEQ ID NO:14, SEQ ID NO:16, SEQ ID NO:18 and SEQ ID NO:42 or sequence variants thereof. The individual sequence elements, e.g. SEQ ID NO:4, SEQ ID NO:8, SEQ ID NO:12, SEQ ID NO:14, SEQ ID NO:16, SEQ ID NO:18 and SEQ ID NO:42 or sequence variants thereof may e.g. be a continuous sequence, or e.g. be separated by nucleotide sequences, for as long as the reading frame of the second ORF is not changed.

Preferably, the inventive vector comprises a first and second ORF, wherein the first and/or second ORF each preferably comprise at least one or more, in particular 1-4, nucleotide sequences selected from the group consisting of nucleotide sequences encoding the HCMV glycoproteins gH, gL, pUL128, pUL130 and pUL131 or sequence variants thereof, i.e. an amino acid sequence according to SEQ ID NO:21, SEQ ID NO:25, SEQ ID NO:3, SEQ ID NO:7 and SEQ ID NO:11 or sequence variants thereof, e.g. nucleotide sequences according to SEQ ID NO:6 and/or SEQ ID NO:10 and/or SEQ ID NO:24 and/or SEQ ID NO:28 and/or SEQ ID NO:30 or sequence variants thereof. Accordingly, the first ORF of the inventive gene expression system as defined above may comprise SEQ ID NO:6 and/or SEQ ID NO:10 and/or SEQ ID NO:24 and/or SEQ ID NO:28 and/or SEQ ID NO:30 or sequence variants thereof, e.g. the first ORF as defined above may comprise SEQ ID NO:6, or SEQ ID NO:10, or SEQ ID NO:24, or SEQ ID NO:28 or SEQ ID NO:30 or sequence variants thereof, or e.g. SEQ ID NO:6 and SEQ ID NO:10 or SEQ ID NO:24, or SEQ ID NO:28 or SEQ ID NO:30 or sequence variants thereof, e.g. the first ORF may comprise SEQ ID NO:22, SEQ ID NO:6, SEQ ID NO:26 or sequence variants thereof, or e.g. SEQ ID NO:22, SEQ ID NO:10, SEQ ID NO:26, or e.g. SEQ ID NO:22, SEQ ID NO:24, SEQ ID NO:26 or sequence variants thereof, or e.g. SEQ ID NO:22, SEQ ID NO:28, SEQ ID NO:26, or e.g. SEQ ID NO:22, SEQ ID NO:30, SEQ ID NO:26 or sequence variants thereof or e.g. SEQ ID NO:26, SEQ ID NO:6, SEQ ID NO:22 or sequence variants thereof, or e.g. SEQ ID NO:26, SEQ ID NO:10, SEQ ID NO:22 or sequence variants thereof, or e.g. SEQ ID NO:26, SEQ ID NO:24, SEQ ID NO:22 or sequence variants thereof, or e.g. SEQ ID NO:26, SEQ ID NO:28, SEQ ID NO:22 or sequence variants thereof, or e.g. SEQ ID NO:26, SEQ ID NO:30, SEQ ID NO:22 or sequence variants thereof. The first ORF of the inventive vector may e.g. further also comprise a signal peptide, in particular for secretion to the extracellular environment, e.g. encoding SEQ ID NO:19 or sequence variants thereof, by e.g. SEQ ID NO:20 or sequence variants thereof, e.g. the first ORF may comprise operably linked a 5′ start codon and SEQ ID NO:20 or a sequence variant thereof. Accordingly, the nucleotide sequence may further comprise a KOZAK sequence as defined above to improve translation initiation of the resulting mRNA.

Accordingly, the second ORF of the inventive vector may preferably comprise at least one or more, in particular 1-4, nucleotide sequences selected from the group consisting of nucleotide sequences encoding the HCMV glycoproteins gH, gL, pUL128, pUL130 and pUL131 or sequence variants thereof, i.e. an amino acid sequence according to SEQ ID NO:21, SEQ ID NO:25, SEQ ID NO:3, SEQ ID NO:7 and SEQ ID NO:11 or sequence variants thereof, e.g. nucleotide sequences according to SEQ ID NO:6 and/or SEQ ID NO:10 and/or SEQ ID NO:24 and/or SEQ ID NO:28 and/or SEQ ID NO:30 or sequence variants thereof. Thus, the second ORF of the inventive gene expression system may e.g. comprise SEQ ID NO:6, or SEQ ID NO:10, or SEQ ID NO:24, or SEQ ID NO:28, or SEQ ID NO:30 or sequence variants thereof, or e.g. SEQ ID NO:6, SEQ ID NO:10 or sequence variants thereof, or e.g. SEQ ID NO:6, SEQ ID NO:24 or sequence variants thereof, or e.g. SEQ ID NO:6, SEQ ID NO:28 or sequence variants thereof, or e.g. SEQ ID NO:6, SEQ ID NO:30 or sequence variants thereof, or e.g. SEQ ID NO:10, SEQ ID NO:24 or sequence variants thereof, or e.g. SEQ ID NO:10, SEQ ID NO:28, or e.g. SEQ ID NO:30 or sequence variants thereof, or e.g. SEQ ID NO:24, SEQ ID NO:28 or sequence variants thereof, or e.g. SEQ ID NO:24, SEQ ID NO:30 or sequence variants thereof.

Accordingly, the first and second ORF of the inventive vector may comprise SEQ ID NO:6 and/or SEQ ID NO:10 and/or SEQ ID NO:24 and/or SEQ ID NO:28 and/or SEQ ID NO:30 or sequence variants thereof, e.g. SEQ ID NO:6, or SEQ ID NO:6, or SEQ ID NO:10, or SEQ ID NO:24, or SEQ ID NO:28, or SEQ ID NO:30 or sequence variants thereof, or e.g. SEQ ID NO:6, SEQ ID NO:10 or sequence variants thereof, or e.g. SEQ ID NO:6, SEQ ID NO:24 or sequence variants thereof, or e.g. SEQ ID NO:6, SEQ ID NO:28 or sequence variants thereof, or e.g. SEQ ID NO:6, SEQ ID NO:30 or sequence variants thereof, or e.g. SEQ ID NO:10, SEQ ID NO:24 or sequence variants thereof, or e.g. SEQ ID NO:10, SEQ ID NO:28 or sequence variants thereof, or e.g. SEQ ID NO:30 or sequence variants thereof, or e.g. SEQ ID NO:24, SEQ ID NO:28 or sequence variants thereof, or e.g. SEQ ID NO:24, SEQ ID NO:30 or sequence variants thereof, or e.g. SEQ ID NO:6, SEQ ID NO:10, SEQ ID NO:24 or sequence variants thereof, or e.g. SEQ ID NO:6, SEQ ID NO:10, SEQ NO:28 or sequence variants thereof, or e.g. NO:6, SEQ ID NO:10, SEQ NO:30 or sequence variants thereof, or e.g. SEQ ID NO:10, SEQ ID NO:24, SEQ ID NO:28 or sequence variants thereof, or e.g. SEQ ID NO:10, SEQ ID NO:24, SEQ ID NO:30 or sequence variants thereof, or e.g. SEQ ID NO:24, SEQ ID NO:28, SEQ ID NO:30 or sequence variants thereof, or e.g. SEQ ID NO:6, SEQ ID NO:24, SEQ ID NO:30 or sequence variants thereof, or e.g. SEQ ID NO:6, SEQ ID NO:28, SEQ ID NO:30 or sequence variants thereof.

According to a more preferred embodiment, a first and second ORF of the inventive vector preferably each comprise at least one nucleotide sequence according to SEQ ID NO:24 and/or SEQ ID NO:28 and/or SEQ ID NO:30 or sequence variants thereof. Accordingly, the first and second ORF of the inventive gene expression system may e.g. each comprise at least one nucleotide sequence according to SEQ ID NO:24, or SEQ ID NO:28, or SEQ ID NO:30 or sequence variants thereof, e.g. the first ORF may comprise SEQ ID NO:24, or SEQ ID NO:28, or SEQ ID NO:30 or sequence variants thereof, while the second ORF may comprise e.g. SEQ ID NO:24 and SEQ ID NO:28 or sequence variants thereof, or e.g. SEQ ID NO:24 and SEQ ID NO:30 or sequence variants thereof, or e.g. SEQ ID NO:28 and SEQ ID NO:30 or sequence variants thereof.

According to an even more preferred embodiment, the vector according to the present invention comprises a first ORF, which comprises operably linked the nucleotide sequence sequences according to SEQ ID NO:20, SEQ ID NO:22 and SEQ ID NO:24 and SEQ ID NO:26, or the nucleotide sequences according to SEQ ID NO:20, SEQ ID NO:36, SEQ ID NO:28 and SEQ ID NO:38, or the nucleotide sequences according to SEQ ID NO:20, SEQ ID NO:36, SEQ ID NO:30 and SEQ ID NO:38, and a second ORF comprises operably linked SEQ ID NO:4, SEQ ID NO:24, SEQ ID NO:8, SEQ ID NO:24, and SEQ ID NO:12, or operably linked SEQ ID NO:4, SEQ ID NO:24, SEQ ID NO:8, SEQ ID NO:24, and SEQ ID NO:12, SEQ ID NO:14, SEQ ID NO:16, SEQ ID NO:18 and SEQ ID NO:42. Accordingly, the first ORF of the inventive gene expression system may comprise operably linked the nucleic acid sequences according to e.g. SEQ ID NO:20, SEQ ID NO:22, SEQ ID NO:24 and SEQ ID NO:26, or e.g. SEQ ID NO:20, SEQ ID NO:36, SEQ ID NO:28 and SEQ ID NO:38, or e.g. SEQ ID NO:20, SEQ ID NO:36, SEQ ID NO:30 and SEQ ID NO:38, or e.g. SEQ ID NO:20, SEQ ID NO:22, SEQ ID NO:6 and SEQ ID NO:26, or e.g. SEQ ID NO:20, SEQ ID NO:22, SEQ ID NO:10 and SEQ ID NO:26, or e.g. SEQ ID NO:20, SEQ ID NO:22, SEQ ID NO:28 and SEQ ID NO:26, or e.g. SEQ ID NO:20, SEQ ID NO:22, SEQ ID NO:30 and SEQ ID NO:26, or e.g. SEQ ID NO:20, SEQ ID NO:36, SEQ ID NO:6 and SEQ ID NO:26, or e.g. SEQ ID NO:20, SEQ ID NO:36, SEQ ID NO:10 and SEQ ID NO:26, or e.g. SEQ ID NO:20, SEQ ID NO:36, SEQ ID NO:24 and SEQ ID NO:26, or e.g. SEQ ID NO:20, SEQ ID NO:36, SEQ ID NO:6 and SEQ ID NO:38, or e.g. SEQ ID NO:20, SEQ ID NO:36, SEQ ID NO:10 and SEQ ID NO:38, or e.g. SEQ ID NO:20, SEQ ID NO:36, SEQ ID NO:24 and SEQ ID NO:38. Accordingly, the second ORF of the inventive gene expression system may comprise operably linked SEQ ID NO:2, SEQ ID NO:4, SEQ ID NO:24, SEQ ID NO:8, SEQ ID NO:24, SEQ ID NO:12, SEQ ID NO:14, SEQ ID NO:16, SEQ ID NO:18 and SEQ ID NO:42, or e.g. SEQ ID L0 NO:20, SEQ ID NO:4, SEQ ID NO:10, SEQ ID NO:32, SEQ ID NO:10, SEQ ID NO:34, and SEQ ID NO:40, or e.g. SEQ ID NO:20, SEQ ID NO:4, SEQ ID NO:24, SEQ ID NO:32, SEQ ID NO:24, SEQ ID NO:34, and SEQ ID NO:40, or e.g. SEQ ID NO:20, SEQ ID NO:4, SEQ ID NO:28, SEQ ID NO:32, SEQ ID NO:28, SEQ ID NO:34, and SEQ ID NO:40, or e.g. SEQ ID NO:20, SEQ ID NO:4, SEQ ID NO:28, SEQ ID NO:32, SEQ ID NO:28, and SEQ ID NO:34, or e.g. SEQ ID NO:20, SEQ ID NO:4, SEQ ID NO:24, SEQ ID NO:32, SEQ ID NO:24, and SEQ ID NO:34, or e.g. SEQ ID NO:20, SEQ ID NO:4, SEQ ID NO:30, SEQ ID NO:32, SEQ ID NO:30, and SEQ ID NO:34, or e.g. SEQ ID NO:20, SEQ ID NO:4, SEQ ID NO:28, SEQ ID NO:8, SEQ ID NO:28, SEQ ID NO:12, SEQ ID NO:14, SEQ ID NO:16, SEQ ID NO:18 and SEQ ID NO:42, or e.g. SEQ ID NO:20, SEQ ID NO:4, SEQ ID NO:30, SEQ ID NO:8, SEQ ID NO:30, SEQ ID NO:12, SEQ ID NO:14, SEQ ID NO:16, SEQ ID NO:18 and SEQ ID NO:42, or e.g. SEQ ID NO:20, SEQ ID NO:4, SEQ ID NO:30, SEQ ID NO:32, SEQ ID NO:30, SEQ ID NO:12, SEQ ID NO:14, SEQ ID NO:16, SEQ ID NO:18 and SEQ ID NO:42, or e.g. SEQ ID NO:4, SEQ ID NO:30, SEQ ID NO:32, SEQ ID NO:30, SEQ ID NO:12, SEQ ID NO:14, SEQ ID NO:16, SEQ ID NO:40 and SEQ ID NO:42, or e.g. SEQ ID NO:20, SEQ ID NO:4, SEQ ID NO:6, SEQ ID NO:32, SEQ ID NO:6, SEQ ID NO:12, SEQ ID NO:14, SEQ ID NO:16, SEQ ID NO:18 and SEQ ID NO:42, or e.g. SEQ ID NO:2, SEQ ID NO:4, SEQ ID NO:6, SEQ ID NO:32, SEQ ID NO:6, SEQ ID NO:34, SEQ ID NO:14, SEQ ID NO:16, SEQ ID NO:18 and SEQ ID NO:42, or e.g. SEQ ID NO:2, SEQ ID NO:4, SEQ ID NO:10, SEQ ID NO:32, SEQ ID NO:10, SEQ ID NO:12, SEQ ID NO:14, SEQ ID NO:16, SEQ ID NO:18 and SEQ ID NO:42, or e.g. SEQ ID NO:2, SEQ ID NO:4, SEQ ID NO:24, SEQ ID NO:32, SEQ ID NO:24, SEQ ID NO:12, SEQ ID NO:14, SEQ ID NO:16, SEQ ID NO:18 and SEQ ID NO:42, or e.g. SEQ ID NO:2, SEQ ID NO:4, SEQ ID NO:28, SEQ ID NO:32, SEQ ID NO:28, SEQ ID NO:12, SEQ ID NO:14, SEQ ID NO:16, SEQ ID NO:18 and SEQ ID NO:42, or e.g. SEQ ID NO:2, SEQ ID NO:4, SEQ ID NO:30, SEQ ID NO:32, SEQ ID NO:30, SEQ ID NO:12, SEQ ID NO:14, SEQ ID NO:16, SEQ ID NO:18 and SEQ ID NO:42, or e.g. SEQ ID NO:2, SEQ 5 ID NO:4, SEQ ID NO:24, SEQ ID NO:8, SEQ ID NO:24, SEQ ID NO:12, or e.g. SEQ ID NO:2, SEQ ID NO:4, SEQ ID NO:6, SEQ ID NO:8, SEQ ID NO:6, and SEQ ID NO:12, or e.g. SEQ ID NO:20, SEQ ID NO:4, SEQ ID NO:10, SEQ ID NO:32, SEQ ID NO:10, and SEQ ID NO:34, or e.g. SEQ ID NO:20, SEQ ID NO:4, SEQ ID NO:10, SEQ ID NO:32, SEQ ID NO:10, and SEQ ID NO:12, or e.g. SEQ ID NO:20, SEQ ID NO:4, SEQ ID NO:24, SEQ ID NO:32, SEQ ID NO:24, and SEQ ID NO:12 or e.g. SEQ ID NO:20, SEQ ID NO:4, SEQ ID NO:28, SEQ ID NO:32, SEQ ID NO:28, and SEQ ID NO:12, or e.g. SEQ ID NO:20, SEQ ID NO:4, SEQ ID NO:30, SEQ ID NO:32, SEQ ID NO:30, and SEQ ID NO:12, preferably e.g. SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 and SEQ ID NO:26, or e.g. SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 and SEQ ID NO:26, or e.g. SEQ ID NO:20, SEQ ID NO:4, SEQ ID NO:28, SEQ ID NO:32, SEQ ID NO:28, SEQ ID NO:34, SEQ ID NO:20, SEQ ID NO:36, SEQ ID NO:28 and SEQ ID NO:38, or e.g. SEQ ID NO:20, SEQ ID NO:4, SEQ ID NO:30, SEQ ID NO:32, SEQ ID NO:30, SEQ ID NO:34, SEQ ID NO:20, SEQ ID NO:36, SEQ ID NO:30 and SEQ ID NO:38, or e.g. SEQ ID NO:20, SEQ ID NO:4, SEQ ID NO:28, SEQ ID NO:32, SEQ ID NO:28, SEQ ID NO:34, SEQ ID NO:14, SEQ ID NO:16, SEQ ID NO:40 and SEQ ID NO:42, SEQ ID NO:20, SEQ ID NO:36, SEQ ID NO:28 and SEQ ID NO:38, or e.g. SEQ ID NO:20, SEQ ID NO:4, SEQ ID NO:30, SEQ ID NO:32, SEQ ID NO:30, SEQ ID NO:34, SEQ ID NO:14, SEQ ID NO:16, SEQ ID NO:40 and SEQ ID NO:42, SEQ ID NO:20, SEQ ID NO:36, SEQ ID NO:30 and SEQ ID NO:38, or e.g. SEQ ID NO:20, SEQ ID NO:4, SEQ ID NO:28, SEQ ID NO:32, SEQ ID NO:28, SEQ ID NO:34, SEQ ID NO:14, SEQ ID NO:16, SEQ ID NO:40, SEQ ID NO:20, SEQ ID NO:36, SEQ ID NO:28 and SEQ ID NO:38, or e.g. by SEQ ID NO:20, SEQ ID NO:4, SEQ ID NO:30, SEQ ID NO:32, SEQ ID NO:30, SEQ ID NO:34, SEQ ID NO:14, SEQ ID NO:16, SEQ ID NO:40, SEQ ID NO:20, SEQ ID NO:36, SEQ ID NO:30 and SEQ ID NO:38.

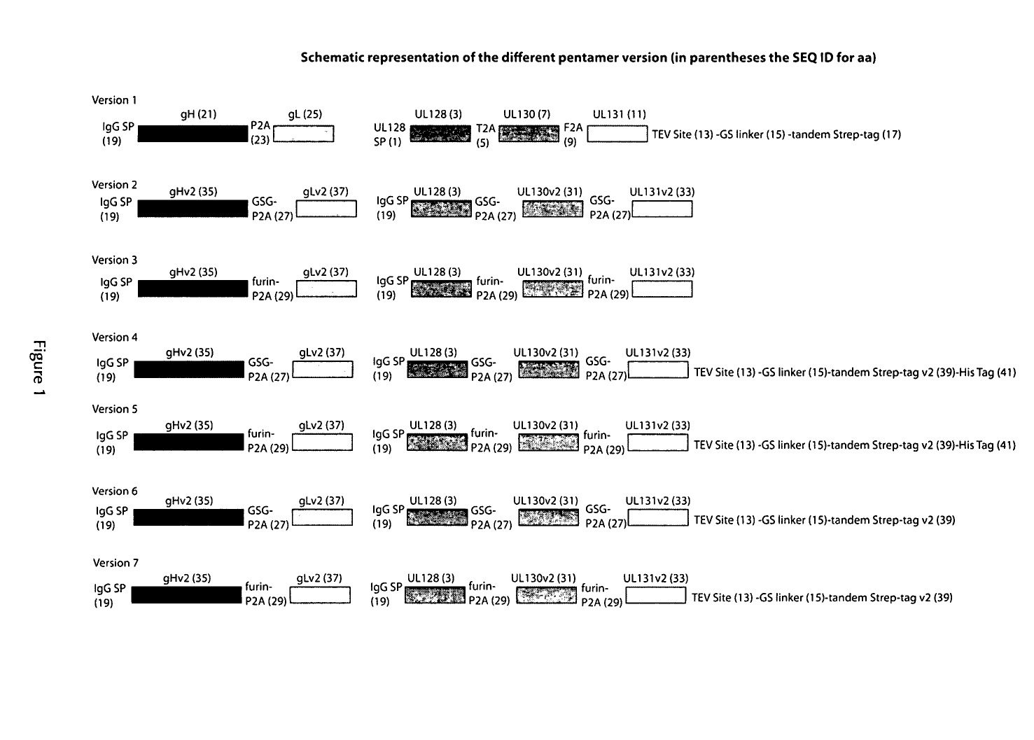

Particularly preferred versions of the construct pentamer according to the present invention are schematically shown in FIG. 1. These particularly preferred pentamer versions are obtained by a vector according to the present invention, which is also particularly preferred and which comprises a transcription system comprising
(i) a first promoter operable in a mammalian cell and operably linked to
(ii) a first open reading frame comprising a nucleotide sequence encoding gH and a nucleotide sequence encoding gL or sequence variants thereof; and
(iii) a second promoter operable in a mammalian cell and operably linked to
(iv) a second open reading frame comprising a nucleotide sequence encoding UL128, a nucleotide sequence encoding UL130 and a nucleotide sequence encoding UL131 or sequence variants thereof.

The vector is preferably a double gene mammalian expression vector as described above, whereby the first and the second promoter are identical, e.g. hCMV-MIE promoter or mCMV promoter.

To obtain “Version 1” shown in FIG. 1, the particularly preferred vector as described above comprises in the first ORF in 5′-3′ direction: a nucleotide sequence encoding the amino acid sequence according to SEQ ID NO:19 or sequence variants thereof, a nucleotide sequence encoding the amino acid sequence according to SEQ ID NO:21 or sequence variants thereof, a nucleotide sequence encoding the amino acid sequence according to SEQ ID NO:23 or sequence variants thereof, and a nucleotide sequence encoding the amino acid sequence according to SEQ ID NO:25 or sequence variants thereof; and in the second ORF in 5′-3′ direction: a nucleotide sequence encoding the amino acid sequence according to SEQ ID NO:1, or sequence variants thereof, a nucleotide sequence encoding the amino acid sequence according to SEQ ID NO:3, or sequence variants thereof, a nucleotide sequence encoding the amino acid sequence according to SEQ ID NO:5, or sequence variants thereof, a nucleotide sequence encoding the amino acid sequence according to SEQ ID NO:7 or sequence variants thereof, a nucleotide sequence encoding the amino acid sequence according to SEQ ID NO:9 or sequence variants thereof, a nucleotide sequence encoding the amino acid sequence according to SEQ ID NO:11 or sequence variants thereof, a nucleotide sequence encoding the amino acid sequence according to SEQ ID NO:13 or sequence variants thereof, a nucleotide sequence encoding the amino acid sequence according to SEQ ID NO:15 or sequence variants thereof, and a nucleotide sequence encoding the amino acid sequence according to SEQ ID NO:17 or sequence variants thereof.

To obtain “Version 2” shown in FIG. 1, the particularly preferred vector as described above comprises in the first ORF in 5′-3′ direction: a nucleotide sequence encoding the amino acid sequence according to SEQ ID NO:19 or sequence variants thereof, a nucleotide sequence encoding the amino acid sequence according to SEQ ID NO:35 or sequence variants thereof, a nucleotide sequence encoding the amino acid sequence according to SEQ ID NO:27 or sequence variants thereof, and a nucleotide sequence encoding the amino acid sequence according to SEQ ID NO:37 or sequence variants thereof; and in the second ORF in 5′-3′ direction: a nucleotide sequence encoding the amino acid sequence according to SEQ ID NO:19, or sequence variants thereof, a nucleotide sequence encoding the amino acid sequence according to SEQ ID NO:3, or sequence variants thereof, a nucleotide sequence encoding the amino acid sequence according to SEQ ID NO:27, or sequence variants thereof, a nucleotide sequence encoding the amino acid sequence according to SEQ ID NO:31 or sequence variants thereof, a nucleotide sequence encoding the amino acid sequence according to SEQ ID NO:27 or sequence variants thereof, and a nucleotide sequence encoding the amino acid sequence according to SEQ ID NO:33 or sequence variants thereof.

To obtain “Version 3” shown in FIG. 1, the particularly preferred vector as described above comprises in the first ORF in 5′-3′ direction: a nucleotide sequence encoding the amino acid sequence according to SEQ ID NO:19 or sequence variants thereof, a nucleotide sequence encoding the amino acid sequence according to SEQ ID NO:35 or sequence variants thereof, a nucleotide sequence encoding the amino acid sequence according to SEQ ID NO:29 or sequence variants thereof, and a nucleotide sequence encoding the amino acid sequence according to SEQ ID NO:37 or sequence variants thereof; and in the second ORF in 5′-3′ direction: a nucleotide sequence encoding the amino acid sequence according to SEQ ID NO:19, or sequence variants thereof, a nucleotide sequence encoding the amino acid sequence according to SEQ ID NO:3, or sequence variants thereof, a nucleotide sequence encoding the amino acid sequence according to SEQ ID NO:29, or sequence variants thereof, a nucleotide sequence encoding the amino acid sequence according to SEQ ID NO:31 or sequence variants thereof, a nucleotide sequence encoding the amino acid sequence according to SEQ ID NO:29 or sequence variants thereof, and a nucleotide sequence encoding the amino acid sequence according to SEQ ID NO:33 or sequence variants thereof.

To obtain “Version 4” shown in FIG. 1, the particularly preferred vector as described above comprises in the first ORF in 5′-3′ direction: a nucleotide sequence encoding the amino acid sequence according to SEQ ID NO:19 or sequence variants thereof, a nucleotide sequence encoding the amino acid sequence according to SEQ ID NO:35 or sequence variants thereof, a nucleotide sequence encoding the amino acid sequence according to SEQ ID NO:27 or sequence variants thereof, and a nucleotide sequence encoding the amino acid sequence according to SEQ ID NO:37 or sequence variants thereof; and in the second ORF in 5′-3′ direction: a nucleotide sequence encoding the amino acid sequence according to SEQ ID NO:19, or sequence variants thereof, a nucleotide sequence encoding the amino acid sequence according to SEQ ID NO:3, or sequence variants thereof, a nucleotide sequence encoding the amino acid sequence according to SEQ ID NO:27, or sequence variants thereof, a nucleotide sequence encoding the amino acid sequence according to SEQ ID NO:31 or sequence variants thereof, a nucleotide sequence encoding the amino acid sequence according to SEQ ID NO:27 or sequence variants thereof, a nucleotide sequence encoding the amino acid sequence according to SEQ ID NO:33 or sequence variants thereof, a nucleotide sequence encoding the amino acid sequence according to SEQ ID NO:13 or sequence variants thereof, a nucleotide sequence encoding the amino acid sequence according to SEQ ID NO:15 or sequence variants thereof, a nucleotide sequence encoding the amino acid sequence according to SEQ ID NO:39 or sequence variants thereof, and a nucleotide sequence encoding the amino acid sequence according to SEQ ID NO:41 or sequence variants thereof.

To obtain “Version 5” shown in FIG. 1, the particularly preferred vector as described above comprises in the first ORF in 5′-3′ direction: a nucleotide sequence encoding the amino acid sequence according to SEQ ID NO:19 or sequence variants thereof, a nucleotide sequence encoding the amino acid sequence according to SEQ ID NO:35 or sequence variants thereof, a nucleotide sequence encoding the amino acid sequence according to SEQ ID NO:29 or sequence variants thereof, and a nucleotide sequence encoding the amino acid sequence according to SEQ ID NO:37 or sequence variants thereof; and in the second ORF in 5′-3′ direction: a nucleotide sequence encoding the amino acid sequence according to SEQ ID NO:19, or sequence variants thereof, a nucleotide sequence encoding the amino acid sequence according to SEQ ID NO:3, or sequence variants thereof, a nucleotide sequence encoding the amino acid sequence according to SEQ ID NO:29, or sequence variants thereof, a nucleotide sequence encoding the amino acid sequence according to SEQ ID NO:31 or sequence variants thereof, a nucleotide sequence encoding the amino acid sequence according to SEQ ID NO:29 or sequence variants thereof, a nucleotide sequence encoding the amino acid sequence according to SEQ ID NO:33 or sequence variants thereof, a nucleotide sequence encoding the amino acid sequence according to SEQ ID NO:13 or sequence variants thereof, a nucleotide sequence encoding the amino acid sequence according to SEQ ID NO:15 or sequence variants thereof, a nucleotide sequence encoding the amino acid sequence according to SEQ ID NO:39 or sequence variants thereof, and a nucleotide sequence encoding the amino acid sequence according to SEQ ID NO:41 or sequence variants thereof.

To obtain “Version 6” shown in FIG. 1, the particularly preferred vector as described above comprises in the first ORF in 5′-3′ direction: a nucleotide sequence encoding the amino acid sequence according to SEQ ID NO:19 or sequence variants thereof, a nucleotide sequence encoding the amino acid sequence according to SEQ ID NO:35 or sequence variants thereof, a nucleotide sequence encoding the amino acid sequence according to SEQ ID NO:27 or sequence variants thereof, and a nucleotide sequence encoding the amino acid sequence according to SEQ ID NO:37 or sequence variants thereof; and in the second ORF in 5′-3′ direction: a nucleotide sequence encoding the amino acid sequence according to SEQ ID NO:19, or sequence variants thereof, a nucleotide sequence encoding the amino acid sequence according to SEQ ID NO:3, or sequence variants thereof, a nucleotide sequence encoding the amino acid sequence according to SEQ ID NO:27, or sequence variants thereof, a nucleotide sequence encoding the amino acid sequence according to SEQ ID NO:31 or sequence variants thereof, a nucleotide sequence encoding the amino acid sequence according to SEQ ID NO:27 or sequence variants thereof, a nucleotide sequence encoding the amino acid sequence according to SEQ ID NO:33 or sequence variants thereof, a nucleotide sequence encoding the amino acid sequence according to SEQ ID NO:13 or sequence variants thereof, a nucleotide sequence encoding the amino acid sequence according to SEQ ID NO:15 or sequence variants thereof, and a nucleotide sequence encoding the amino acid sequence according to SEQ ID NO:39 or sequence variants thereof.

To obtain “Version 7” shown in FIG. 1, the particularly preferred vector as described above comprises in the first ORF in 5′-3′ direction: a nucleotide sequence encoding the amino acid sequence according to SEQ ID NO:19 or sequence variants thereof, a nucleotide sequence encoding the amino acid sequence according to SEQ ID NO:35 or sequence variants thereof, a nucleotide sequence encoding the amino acid sequence according to SEQ ID NO:29 or sequence variants thereof, and a nucleotide sequence encoding the amino acid sequence according to SEQ ID NO:37 or sequence variants thereof; and in the second ORF in 5′-3′ direction: a nucleotide sequence encoding the amino acid sequence according to SEQ ID NO:19, or sequence variants thereof, a nucleotide sequence encoding the amino acid sequence according to SEQ ID NO:3, or sequence variants thereof, a nucleotide sequence encoding the amino acid sequence according to SEQ ID NO:29, or sequence variants thereof, a nucleotide sequence encoding the amino acid sequence according to SEQ ID NO:31 or sequence variants thereof, a nucleotide sequence encoding the amino acid sequence according to SEQ ID NO:29 or sequence variants thereof, a nucleotide sequence encoding the amino acid sequence according to SEQ ID NO:33 or sequence variants thereof, a nucleotide sequence encoding the amino acid sequence according to SEQ ID NO:13 or sequence variants thereof, a nucleotide sequence encoding the amino acid sequence according to SEQ ID NO:15 or sequence variants thereof, and a nucleotide sequence encoding the amino acid sequence according to SEQ ID NO:39 or sequence variants thereof.

According to a second aspect the present invention provides for a gene expression system, which comprises at least one mammalian cell and a vector according to the invention, e.g. as described above, for expressing HCMV glycoproteins in said mammalian cell, wherein the vector comprises a transcription system. The inventive gene expression system thus comprises at least one mammalian cell, e.g. if at least one mammalian cell of the inventive gene expression system is grown in suspension, the inventive gene expression system may comprise least one mammalian cell, or at least 10, or at least 100, or at least 1000, or at least about 10,000 cells, or of at least about 10⁵, 10⁶, 10⁷, 10⁸, 10⁹, 10¹⁰, 10¹¹, 10¹² mammalian cells, or e.g. of about 10³ cells/ml, or of about 10⁴ cells/ml, to about 10⁹ cells/ml, e.g. 10⁵ cells/ml, 10⁶ cells/ml, 10⁷ cells/ml, 10⁸ cells/ml, or of about 2.5×10² cells/ml, 3×10² cells/ml, 5×10² cells/ml, 10³ cells/ml, 1.25×10³ cells/ml, 2.5×10³ cells/ml, 5×10³ cells/ml, 7.5×10³ cells/ml, 1×10⁴ cells/ml, 2.5×10⁴ cells/ml, 5×10⁴ cells/ml, 7.5×10⁴ cells/ml, 1×10⁵ cell/ml to about 2.5×10⁵ ells/ml, 5×10⁵ cells/ml, 7.5×10⁵ cells/ml, 1×10⁶ cells/ml, 2.5×10⁶ cells/ml, 5×10⁶ cells/ml, 7.5×10⁶ cells/ml, 1×10⁷ cells/ml, 5×10⁵ cells/ml, 1×10⁸ cells/ml, 2.5×10⁸ cells/ml, 5×10⁸ cells/ml, 1×10⁹ cells/ml. Alternatively, the inventive gene expression system may comprise e.g. at least 10² cells/cm² to about 10⁶ cells/cm², if the at least one mammalian cell is grown on a solid support, e.g. 10², 10³, 10⁴, 10⁵ or 10⁶ cells/cm², or e.g. of about 1×10² cells/cm², 2.5×10² cells/cm², 5×10² cells/cm², 7.5×10² cells/cm², 1×10³ cells/cm² to about 1×10⁵ cells/cm², 2.5×10⁵ cells/cm², 5×10⁵ cells/cm², 7.5×10⁵ cells/cm², or e.g. 2.5×10³ cell/cm² to 2.5×10⁴ cell/cm².

In a more specific embodiment, the at least one mammalian cell comprised in the gene expression system according to the invention is selected from the group comprising BHK, DUXB11, CHO-DG44, CHO-K1, CHO-K1SV, CHO-S, CHO-DXB11, CHO-K1SV GS knock-out (CHO-K1SV KO), CAP, PER.C6, NS0, Sp2/0, HEK293 T, HEK 293-F, HEK 6E, HEK293 EBNA, CAP-T, HELA, CVI, COS, R1610, BALBC/3T3, HAK, BFA-1c1 BPT, RAJI, HT-1080, HKB-11. For example, the inventive gene expression system may comprise at least one mammalian cell as defined above, preferably the at least one mammalian cell is selected from the group comprising CHO-DG44, CHO-K1, CHO-K1SV, CHO-S, CHO-DXB11, CHO-K1SV GS knock-out (CHO-K1SV KO) cells. Accordingly, the at least one mammalian cell of the inventive gene expression system as defined above may be a CHO-DG44 cell, or e.g. a CHO-K1 cell, or e.g. a CHO-K1SV cell, or e.g. a CHO-S cell, or e.g. a CHO-DXB11 cell, or e.g. a CHO-K1SV GS knock-out (CHO-K1SV KO) cell.

In the inventive gene expression system it is preferred that the mammalian cell is transfected by the vector according to the invention. The term “transfected” or “transfection” as used herein refers to deliberately introducing nucleic acids, e.g. the inventive vector, into cells. In general, the transfection may be transient, i.e. the introduced nucleic acid is usually not integrated in the nuclear genome and the transfected genetic material is only transiently expressed, or stable, whereby the introduced nucleic acid is integrated in the genome of the host cell (also referred to as a“Nucleofection®”, whereby Nucleofection® typically refers to an electroporation-based transfection method that enables DNA or RNA to enter directly the nucleus and the cytoplasm). It is particularly preferred that the mammalian cell is stably transfected, in particular nucleofected, by the inventive vector.

Nucleofection® is based on the physical method of electroporation and typically uses a combination of electrical parameters, generated by a device called Nucleofector®, with cell-type specific reagents. The substrate, e.g. the vector, is transferred directly into the cell nucleus and the cytoplasm. Thus, Nucleofection® is a non-viral transfection method enabling efficient gene transfer, which is otherwise restricted to the use of viral vectors, which typically involve disadvantages such as safety risks, lack of reliability, and high cost.

In particular, the vector according to the present invention also ensures equimolar expression of the subunits upon stable transfection, i.e. upon integration into the host genome. Thereby, the one or more open reading frames comprised by a single vector are typically integrated into the same genomic site having the same transcriptional activity. Accordingly, the nucleotide sequences encoding the five subunits comprised by a single vector according to the present invention are typically integrated into the same genomic site upon stable transfection resulting in a balanced expression. In contrast, if more than one vector is used, different open reading frames located on the different vectors are typically integrated into different genomic sites. However, in different genomic sites the level of chromatin accessibility for transcription may be different, resulting in expression differences of the different ORFS derived from the different vectors.

Accordingly, the present invention also provides a stable cell line secreting a HCMV pentamer comprising the amino acid sequences according to SEQ ID NO:3, SEQ ID NO:7, SEQ ID NO:11, SEQ ID NO:21 and SEQ ID NO:25, or sequence variants thereof, wherein said stable cell line is obtainable by transfection, preferably by Nucleofection®, of at least one mammalian cell with a vector according to the present invention.

The stable cell line may be obtained by transfection, preferably by Nucleofection®, for example according to the Lonza system, e.g. as described herein, by using the Nucleofector® Technology. For example, a cell-type specific Nucleofector® Kit may be used.

Such a stable cell line according to the present invention, which secretes the HCMV pentamer comprising the amino acid sequences according to SEQ ID NO:3, SEQ ID NO:7, SEQ ID NO:11, SEQ ID NO:21 and SEQ ID NO:25, whereby a desired 1:1:1:1:1 stoichiometry of the subunits is enabled by the vector according to the present invention, is suitable for large scale HCMV pentamer production, in particular since the HCMV pentamer is secreted, in particular into the supernatant of the cell culture. Thus, with the stable cell line according to the present invention only the supernatant needs to be harvested to obtain a HCMV pentamer with a desired 1:1:1:1:1 stoichiometry of the subunits.

Preferably, in the stable cell line according to the present invention the at least one mammalian cell is selected from the group consisting of BHK, DUXB11, CHO-DG44, CHO-K1, CHO-K1SV, CHO-S, CHO-DXB11, CHO-K1SV GS knock-out (CHO-K1SV KO), CAP, PER.C6, NS0, Sp2/0, HEK293 T, HEK 293-F, HEK 6E, HEK293 EBNA, CAP-T, HELA, CVI, COS, R1610, BALBC/3T3, HAK, BFA-1c1BPT, RAJI, HT-1080, and HKB-11, preferably the at least one mammalian cell is selected from the group consisting of CHO-DG44, CHO-K1, CHO-K1SV, CHO-S, CHO-DXB11, and CHO-K1SV GS knock-out (CHO-K1SV KO), more preferably the at least one mammalian cell is selected from the group consisting of CHO-K1SV and CHO-K1SV GS knock-out (CHO-K1SV KO).

In a third aspect, the present invention provides for a soluble protein complex, which is obtainable by the inventive gene expression system as described above or by a stable cell line according to the present invention as described above, wherein it is preferred that the protein complex comprises the amino acid sequences according to SEQ ID NO:3, SEQ ID NO:7, SEQ ID NO:11, SEQ ID NO:21 and SEQ ID NO:25 or sequence variants thereof, or SEQ ID NO:45 or sequence variants thereof, or SEQ ID NO:47 or sequence variants thereof, or SEQ ID NO:49 or sequence variants thereof. Accordingly, the inventive soluble protein complex obtainable by the inventive gene expression system as disclosed above or by a stable cell line according to the present invention as described above may comprise the amino acid sequences according to SEQ ID NO:3, SEQ ID NO:7, SEQ ID NO:11, SEQ ID NO:21 and SEQ ID NO:25 or sequence variants thereof, or SEQ ID NO:45 or sequence variants thereof, or SEQ ID NO:47 or sequence variants thereof, or SEQ ID NO:49 or sequence variants thereof, e.g. HCMV proteins UL128, UL130, UL131, gH and gL, which may be encoded by e.g. the nucleotide sequences according to SEQ ID NO:4, SEQ ID NO:8, SEQ ID NO:12, SEQ ID NO:22 and SEQ ID NO:26 or sequence variants thereof, or e.g. by SEQ ID NO:46 or sequence variants thereof, or by e.g. SEQ ID NO:48 or sequence variants thereof, or by e.g. SEQ ID NO:50 or sequence variants thereof.

The term “obtainable” as used herein in the context of the inventive soluble protein complex as disclosed above shall mean that the polypeptide encoded by the nucleotide sequence may be produced by the at least one mammalian cell as disclosed above, preferably by the stable cell line as described above, in which the nucleotide sequences according to the invention, are present, e.g. the nucleotide sequences may be comprised on an inventive expression vector or the nucleotide sequences may be integrated into the genome of the mammalian cell, e.g. by Nucleofection®.

As used within the context of the present invention, e.g. in the context of the inventive gene expression system, the term “protein complex” (herein also referred to as “HCMV pentamer”) refers to a composite unit that is a combination of two or more proteins formed by interaction between the proteins. Typically, but not necessarily, a “protein complex” is formed by the binding and/or interaction of two or more proteins through specific, non-covalent binding interactions.

The protein complex may also be formed by e.g. covalent linkage of the individual proteins of the complex, such as e.g. by a peptide bond or by means of a peptide linker sequence, which via peptide bonds joins two proteins. For example, two or more proteins, e.g. two, three, four or five (e.g all of the) proteins of the inventive soluble protein complex comprising gH, gL, pUL128, pUL130 and pUL131 may be linked via peptide linker. Ideally, the peptide linker for use with the inventive soluble protein complex is of sufficient length and provides sufficient flexibility such that it does not interfere with the folding and/or assembly of the protein complex, such that the conformation of the inventive soluble protein complex is retained. For example, the linker sequence may comprise the amino acid sequence according to SEQ ID NO:15 or sequence variants thereof, or e.g. may comprise the amino acid sequence GSTSGSGXPGSGEGSTKG (SEQ ID NO:51) as disclosed in WO1994/012520, whereby X represents a charged amino acid, or. g. the amino acid sequence Ser-Ser-Ser-Ser-Gly as disclosed in U.S. Pat. No. 5,525,491, or e.g. Gly-Gly-Gly-Gly-Ser-Gly-Gly-Gly-Gly-Ser-Gly-Gly (SEQ ID NO:52) as disclosed in WO2002046227, or e.g. GGGGSGGGGSGGGGSGGGGS (SEQ ID NO:53), or e.g. GGGGSGGGGSGGGGS (SEQ ID NO:54), or e.g. GVGGSGGGGSGGGGS (SEQ ID NO:55) as disclosed in WO2007/136778 or sequence variants thereof. The inventive soluble protein complex may thus comprise the proteins gH, gL, UL128, UL130 and UL131 linked to each other by means of any of e.g. the above sequences, e.g. the HCMV surface glycoproteins, or sequence variants thereof as disclosed in the present invention, may be in the order of e.g. gH-SEQ ID NO:15-gL-SEQ ID NO:15-UL128-SEQ ID NO:15-UL130-SEQ ID NO:15-UL131, or e.g. gH-GGGGSGGGGSGGGGS-gL-GGGGSGGGGSGGGGS-UL128-GGGGSGGGGSGGGGS-UL130-GGGGSGGGGSGGGGS-UL131. The peptide linkers as disclosed above are typically encoded as part of a first and second ORF of the inventive transcription system and the corresponding nucleotide sequences encoding the peptide linker as disclosed above are located in frame between two, e.g. between the 3′ and of a first and the 5′ end of a second nucleotide sequence encoding one of the HCMV surface glycoproteins as disclosed above, or sequence variants thereof, as disclosed in the present invention. However, it is preferred that the hCMV pentamer subunits as described herein are not linked by a peptide linker, since the antigenic sites present on the subunits, which are linked, may be less accessible for an antibody due to the linkage and this may result in poorer recognition of the antigenic sites on the linked subunits by an antibody, in particular by an antibody specifically binding to the relevant antigenic site. For example, the use of the peptide linker sequences, or their corresponding nucleotide sequence, may be comprised in a single ORF of a vector of the inventive gene expression system, which may e.g. result in the translation of a single, self-processing polypeptide, if nucleotide sequences (e.g. SEQ ID NO:6, 10, 24, 28 or 30 or sequence variants thereof) encoding the self-processing peptides as disclosed above are present in the ORF. For example, the two or more proteins of the inventive soluble protein complex can be covalently linked by e.g. disulfide bonds, which may result in a stabilization of the protein complex. Non-covalent binding interactions as referred to above may include e.g. van der Waals interactions, or e.g. ionic interactions between differently charged amino acid residues.

More specifically, the present invention provides for a soluble protein complex, which is obtainable by the inventive gene expression system as defined above or by a stable cell line according to the present invention as described above, wherein the protein complex may comprise the amino acid sequences according to SEQ ID NO:19, SEQ ID NO:21, SEQ ID NO:23, SEQ ID NO:25, SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17 or sequence variants thereof. Accordingly, the soluble protein complex according to the invention obtainable by the inventive gene expression system as defined above or by a stable cell line according to the present invention as described above may comprise the amino acid sequences according to SEQ ID NO:19, SEQ ID NO:21, SEQ ID NO:23, SEQ ID NO:25, SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17 or sequence variants thereof, or e.g. the inventive soluble protein complex may comprise the amino acid sequences encoded by nucleotide sequences SEQ ID NO:20, SEQ ID NO:22, SEQ ID NO:24, SEQ ID NO:26, SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18 or sequence variants thereof.

Furthermore, the present invention provides for a soluble protein complex, which is obtainable by the inventive gene expression system as defined above or by a stable cell line according to the present invention as described above, wherein the protein complex may comprise the amino acid sequences according to SEQ ID NO:19, SEQ ID NO:35, SEQ ID NO:27, SEQ ID NO:37, SEQ ID NO:19, SEQ ID NO:3, SEQ ID NO:27, SEQ ID NO:31, SEQ ID NO:27, SEQ ID NO:3 or sequence variants thereof.

Also, the inventive soluble protein complex obtainable by the inventive gene expression system or by a stable cell line according to the present invention as described above may comprise the amino acid sequences according to SEQ ID NO:19, SEQ ID NO:35, SEQ ID NO:29, SEQ ID NO:37, SEQ ID NO:19, SEQ ID NO:3, SEQ ID NO:29, SEQ ID NO:31, SEQ ID NO:29, SEQ ID NO:33 or sequence variants thereof.

Moreover, the inventive soluble protein complex obtainable by a gene expression system according to the invention or by a stable cell line according to the present invention as described above may comprise the amino acid sequences according to SEQ ID NO:19, SEQ ID NO:35, SEQ ID NO:27, SEQ ID NO:37, SEQ ID NO:19, SEQ ID NO:3, SEQ ID NO:27, SEQ ID NO:31, SEQ ID NO:27, SEQ ID NO:33, SEQ ID NO:13, SEQ ID NO:15, SEQ ID NO:39, SEQ ID NO:41 or sequence variants thereof. The inventive soluble protein complex may also comprise the amino acid sequences according to SEQ ID NO:19, SEQ ID NO:35, SEQ ID NO:27, SEQ ID NO:37, SEQ ID NO:19, SEQ ID NO:3, SEQ ID NO:27, SEQ ID NO:31, SEQ ID NO:27, SEQ ID NO:33, SEQ ID NO:13, SEQ ID NO:15, SEQ ID NO:39, or e.g. according to SEQ ID NO:19, SEQ ID NO:35, SEQ ID NO:29, SEQ ID NO:37, SEQ ID NO:19, SEQ ID NO:3, SEQ ID NO:29, SEQ ID NO:31, SEQ ID NO:29, SEQ ID NO:33, SEQ ID NO:13, SEQ ID NO:15, SEQ ID NO:39 or sequence variants thereof.

More specifically, the inventive protein complex obtainable by a gene expression system according to the invention or by a stable cell line according to the present invention as described above may comprise the amino acid sequences according to SEQ ID NO:43, or SEQ ID NO:45, or SEQ ID NO:47, or SEQ ID N049 or sequence variants thereof.

Preferably, the proteins, which comprise the amino acid sequences encoding the HCMV glycoproteins gH, gL, pUL128, pUL130 and pUL131 or sequence variants thereof, e.g. the amino acid sequences according to SEQ ID NO:3, SEQ ID NO:7, SEQ ID NO:11, SEQ ID NO:21 and SEQ ID NO:25 are present in equal corresponding amounts in the inventive soluble protein complex, e.g. the relative ratio of e.g. the number (moles) of each of the proteins comprised in the inventive soluble protein complex is an integer, whereby the integer may be e.g. 1, or e.g. 2, or e.g. 3, or e.g. 4, preferably the integer of the ratio of the relative abundance of e.g. gH:gL:UL128:UL130:UL131 is 1. For example, the inventive soluble protein complex may comprise the proteins, which comprise the amino acid sequences according to SEQ ID NO:3, SEQ ID NO:7, SEQ ID NO:11, SEQ ID NO:21 and SEQ ID NO:25 in equal stoichiometric amounts, e.g. the inventive soluble protein complex comprises the HCMV proteins pUL128, pUL130, pUL131, gH and gL in a molar ratio of 1:1:1:1:1. The term “molar ratio” as used with the inventive soluble protein complex refers to ratio of moles of each of the proteins comprising the amino acid sequences encoding the HCMV glycoproteins gH, gL, pUL128, pUL130 and pUL131 or sequence variants thereof, e.g. the amino acid sequences according to SEQ ID NO:3, SEQ ID NO:7, SEQ ID NO:11, SEQ ID NO:21 and SEQ ID NO:25, e.g. the inventive soluble protein complex comprises the same number of each of the proteins. Accordingly, the inventive soluble protein complex may also comprise equal stoichiometric amounts of e.g. sequence variants of pUL128, pUL130, pUL131, gH and gL, such as e.g. SEQ ID NO:3, SEQ ID NO:31, SEQ ID NO:11, SEQ ID NO:21 and SEQ ID NO:25 or sequence variants thereof, or e.g. SEQ ID NO:3, SEQ ID NO:7, SEQ ID NO:33, SEQ ID NO:21 and SEQ ID NO:25 or sequence variants thereof, or e.g. SEQ ID NO:3, SEQ ID NO:7, SEQ ID NO:11, SEQ ID NO:35 and SEQ ID NO:25 or sequence variants thereof, or e.g. SEQ ID NO:3, SEQ ID NO:7, SEQ ID NO:11, SEQ ID NO:21 and SEQ ID NO:37 or sequence variants thereof, or e.g. SEQ ID NO:3, SEQ ID NO:31, SEQ ID NO:33, SEQ ID NO:21 and SEQ ID NO:25 or sequence variants thereof, or e.g. SEQ ID NO:3, SEQ ID NO:31, SEQ ID NO:33, SEQ ID NO:35 and SEQ ID NO:25 or sequence variants thereof, or e.g. SEQ ID NO:3, SEQ ID NO:31, SEQ ID NO:33, SEQ ID NO:35 and SEQ ID NO:37 or sequence variants thereof, or e.g. SEQ ID NO:3, SEQ ID NO:31, SEQ ID NO:33, SEQ ID: NO:21 and SEQ ID NO:37 or sequence variants thereof, or e.g. SEQ ID NO:3, SEQ ID NO:31, SEQ ID NO:11, SEQ ID NO:35 and SEQ ID NO:37 or sequence variants thereof, or e.g. SEQ ID NO:3, SEQ ID NO:7, SEQ ID NO:33, SEQ ID NO:35 and SEQ ID NO:37 or sequence variants thereof.

In a particularly preferred embodiment, the inventive soluble protein complex as disclosed above is used as a vaccine. Accordingly, the inventive soluble protein complex as described above may be used as a vaccine. As used herein, the term “vaccine” refers to a formulation which contains the inventive soluble protein complex as disclosed above, which is in a form that is capable of being administered to e.g. a mammal, preferably a human, and which induces an immune response sufficient to induce a therapeutic immunity to prevent, or ameliorate an infection and/or to reduce at least one symptom of an infection and/or to enhance the efficacy of another dose of the inventive soluble protein complex. The term “immune response” as used in the context of the inventive use of the soluble protein complex according to the invention refers to both the humoral immune response and the cell-mediated immune response. The humoral immune response involves the stimulation of the production of antibodies by B lymphocytes that, for example, neutralize infectious agents, such as e.g. viruses, e.g. HCMV, block infectious agents from e.g. entering cells, block replication of said infectious agents, and/or protect host cells from infection and destruction. The cell-mediated immune response is usually mediated by T-lymphocytes and/or other cells, such as macrophages, against an infectious agent, e.g. viruses such as HCMV, exhibited by a vertebrate (e.g., a human), that prevents or ameliorates infection or reduces at least one symptom thereof.

In a fourth aspect, the present invention provides for a vaccine composition, which comprises the inventive soluble protein complex as defined above and optionally one or more pharmaceutically active components. The term “pharmaceutically active component” refers to any compound or composition which, when administered to a human or animal induces a desired pharmacologic, immunogenic, and/or physiologic effect by local and/or systemic action. In one embodiment, the inventive vaccine composition may comprise optionally an inactive carrier (vaccine excipient), such as e.g. aluminium salts, egg protein, formaldehyde, monosodium glutamate, or e.g. carbohydrates, including, but not limited to, sorbitol, mannitol, starch, sucrose, dextran, glutamate or glucose, or e.g. proteins, including, but not limited to, dried milk, serum albumin, casein.

Preferably, the vaccine composition according to the invention comprises one or more adjuvants selected from the group comprising mineral salts, surface-active agents, microparticles, cytokines, hormones, antigen constructs, polyanions, polyacrylics, or water-in-oil emulsions. Accordingly, the inventive vaccine composition may comprise one or more, e.g. two, three, four or more adjuvants in addition to the inventive soluble protein complex as disclosed above. The term “adjuvant,” as used herein, refers to compounds which, when administered to an individual, such as e.g. a human, or tested in vitro, increase the immune response to an antigen, such as the inventive soluble protein complex, in the individual or test system to which said antigen is administered. The use of an adjuvant typically enhances the immune response of the individual to the antigen (e.g. the inventive soluble protein complex as disclosed above) by rendereing the antigen more strongly immunogenic. The adjuvant effect may also enable the use of a lower the dose of antigen necessary to achieve an immune response in said individual, e.g. a lower dose of the inventive vaccine composition may be required to achieve the desired immune response.

More specifically, the inventive vaccine composition may comprise one or more adjuvants selected from the group comprising mineral salts, surface-active agents, microparticles, cytokines, hormones, antigen constructs, polyanions, polyacrylics, or water-in-oil emulsions. Accordingly, the inventive vaccine composition may comprise one more adjuvants, e.g. one, two, three, four, five, six, seven, eight, nine, or ten or more adjuvants. For example the inventive vaccine composition may comprise one, two, three, four, five, six, seven, eight, nine, or ten or more adjuvants selected from aluminum (“Alum”), aluminum hydroxide, aluminum phosphate, calcium phosphate, nonionic block polymer surfactants, virosomes, Saponin (QS-21), meningococcal outer membrane proteins (Proteosomes), immune stimulating complexes (ISCOMs), Cochleates Dimethyl dioctadecyl ammonium bromide (DDA), Avridine (CP20,961), vitamin A, vitamin E, cell wall skeleton of Mycobacterium phlei (Detox®), muramyl dipeptides and tripeptides, Threonyl MDP (SAF-1), Butyl-ester MDP (Murabutide®), Dipalmitoyl phosphatidylethanolamine MTP, Monophosphoryl lipid A, Klebsiella pneumonia glycoprotein, Bordetella pertussis, Bacillus Calmette-Gurin, Vibrio cholerae and Escherichia coli heat labile enterotoxin, Trehalose dimycolate, CpG oligodeoxynucleotides, Interleukin-2, Interferon-γ, Interferon-β, granulocyte-macrophage colony stimulating factor, dehydroepiandrosterone, Flt3 ligand, 1,25-dihydroxy vitamin D3, Interleukin-1, Interleukin-6, Interleukin-12, human growth hormone, β2-microglobulin, lymphotactin, Polyanions, e.g. Dextran, double-stranded polynucleotides, polyacrylics, e.g. polymethylmethacrylate, acrylic acid crosslinked with allyl sucrose (Carbopol 934P), or e.g N-acetyl-glucosamine-3yl-acetyl-L-alanyl-D-isoglutamine (CGP-11637), gamma inulin+aluminum hydroxide (Algammulin), human dendritic cells, lysophosphatidyl glycerol, stearyl tyrosine, tripalmitoyl pentapeptide, Carbopol 974P NF polymer, water-in-oil emulsions, mineral oil (Freund's incomplete), vegetable oil (peanut oil), squalene and squalane, oil-in-water emulsions, Squalene+Tween-80+Span 85 (MF59), or e.g. liposomes, or e.g. biodegradable polymer microspheres, lactide and glycolide, polyphosphazenes, beta-glucan, or e.g. proteinoids. A list of typically used vaccine adjuvants may also be found in e.g. “Vaccine Adjuvants”, edited by D. T. O'Hogan, Humana Press 2000. The adjuvant comprised in the inventive vaccine composition may also include e.g. a synthetic derivative of lipid A, some of which are TLR-4 agonists, and include, but are not limited to: OM174 (2-deoxy-6-o-[2-deoxy-2-[(R)-3-dodecanoyloxytetra-decanoylamino]-4-o-phosphono-D-D-glucopyranosyl]-2-[(R)-3-hydroxy-tetradecanoylamino]-p-D-glucopyranosyldi hydrogen-phosphate), (WO 95/14026) OM 294 DP (3S,9R)-3˜[(R)-dodecanoyloxytetradecanoylam, [(R)-3-hydroxytetradecanoylamino]decan-1,10-diol,1, 10-bis(dihydrogenophosphate) (WO 99/64301 and WO 00/0462) OM 197 MP-Ac DP(35-,9R)-3-D(R)-dodecanoyl-decanoylamino]decan-1,10-diol,1-dihydrogenophosphate-10-(6-aminohexanoate) (WO 01/46127). For example the inventive pharmaceutical composition may comprise only one of the above adjuvants, or e.g. two of the above adjuvants, e.g. combination adjuvants such as e.g. Alum and MPL, or Oil-in-water emulsion and MPL and QS-21, or liposomes and MPL and QS21.

It is particularly preferred that the vaccine composition according to the invention comprises an adjuvant selected from the group comprising Alum, Ribi (Monophosphoryl lipid A, MPL), or MF59. Accordingly, the inventive vaccine composition may comprise Alum, or Ribi (Monophosphoryl lipid A, MPL), or MF59, or e.g. Alum and Ribi, or e.g. Alum and MF59, or e.g. Ribi and MF59.

The inventive vaccine composition may be formulated as a liquid formulation, or alternatively and as a preferred embodiment as a lyophilized formulation. The term “liquid formulation” as used for the inventive vaccine composition refers to a water-based formulation, in particular, a formulation that is an aqueous solution. The liquid composition may e.g. further comprise ethanol, or e.g. non-ionic detergents, or e.g. anti-oxidants, such as oxygen scavengers to prevent oxidation of the inventive vaccine composition, e.g. vitamin E, or e.g. vitamin C. The water for use with the inventive liquid vaccine composition may e.g. be USP-grade water for injection. The inventive liquid vaccine composition formulation may for example also consist of, or comprise an emulsion. An emulsion comprises a liquid suspended in another liquid, typically with the aid of an emulsifier. The inventive liquid vaccine composition may also e.g. be a microemulsion, which is a thermodynamically stable solution that is clear upon visual inspection.

Preferably, the inventive vaccine composition may be provided as a lyophilized formulation. The term “lyophilized formulation” as used with the inventive vaccine composition means a freeze-dried formulation prepared by the processes known in the art, such as e.g. those provided in “Cryopreservation and Freeze-Drying Protocols” (2007), JG Day, GN Stacey (eds)., Springer, ISBN 978-1-58829-377-0, and comprising as essential ingredient the soluble protein complex according to the invention.

More specifically, the inventive vaccine composition may comprise a buffer selected from the group of phosphate buffer, Na-acetate buffer, Tris buffer, MOPS buffer, preferably the buffer is a phosphate buffer. Accordingly, the inventive vaccine composition may comprise a phosphate buffer, or a Na-acetate buffer, or a Tris buffer, or a MOPS buffer, preferably the inventive vaccine composition comprises a phosphate buffer. For example, the inventive vaccine composition may comprise a Na-acetate buffer in a concentration of about 0.1 mM to about 500 mM, or of about 1 mM to about 250 mM, or of about 10 mM to about 125 mM, or of about 25 mM to about 100 mM, or of about 50 mM to about 75 mM, or of about 60 mM to about 70 mM, or of about 7.5 mM, 10 mM, 12.5 mM, 15 mM, 20 mM, 22.5 mM, 25 mM, 27.5 mM, 30 mM, 32.5 mM, 35 mM, 40 mM, 45 mM, 50 mM, 55 mM, 60 mM, 65 mM, 70 mM, 75 mM, 80 mM, 85 mM, 90 mM, 95 mM, 100 mM to about 125 mM, 130 mM, 135 mM, 137 mM, 140 mM, 145 mM, 150 mM, 155 mM, 160 mM, 165 mM, 170 mM, 175 mM, 180 mM, 185 mM, 190 mM, 195 mM, 200 mM, or e.g. about 1 mM, 2 mM, 3 mM, 4 mM, 5 mM, 7.5 mM, 10 mM, 12.5 mM, 15 mM, 17.5 mM, 20 mM, 22.5 mM, 25 mM, 27.5 mM, 30 mM, 32.5 mM, 35 mM, 40 mM, 45 mM, 50 mM, 55 mM, 60 mM, 65 mM, 70 mM, 75 mM, 80 mM, 85 mM, 90 mM, 95 mM, 100 mM, 125 mM, 150 mM, 200 mM, 250 mM, or about 500 mM. The inventive vaccine composition may also comprise a Tris buffer (tris(hydroxymethyl)aminomethane), in the above concentrations, or e.g. a 3-(N-morpholino)propanesulfonic acid) (MPOS) buffer in the above concentrations, or e.g. a (4-(2-hydroxyethyl)-1-piperazineethanesulfonic acid) (HEPES) buffer in the above concentrations, or e.g. a 2-(N-morpholino)ethanesulfonic acid (MES) buffer in the above concentrations, or e.g. a N-cyclohexyl-3-aminopropanesulfonic acid (CAPS) buffer in the above concentrations. According to a preferred embodiment, the inventive vaccine composition comprises a phosphate buffer. Accordingly, the total phosphate concentrations for the buffer may be from about 5 mM to about 500 mM, or from about 7.5 mM, 10 mM, 12.5 mM, 15 mM, 20 mM, 22.5 mM, 25 mM, 27.5 mM, 30 mM, 32.5 mM, 35 mM, 40 mM, 45 mM, 50 mM, 55 mM, 60 mM, 65 mM, 70 mM, 75 mM, 80 mM, 85 mM, 90 mM, 95 mM, 100 mM to about 125 mM, 130 mM, 135 mM, 137 mM, 140 mM, 145 mM, 150 mM, 155 mM, 160 mM, 165 mM, 170 mM, 175 mM, 180 mM, 185 mM, 190 mM, 195 mM, 200 mM, or e.g. 7.5 mM, 10 mM, 12.5 mM, 15 mM, 20 mM, 22.5 mM, 25 mM, 27.5 mM, 30 mM, 32.5 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 137 mM, 140 mM, 145 mM, 150 mM, 155 mM, 160 mM, 165 mM, 170 mM, 175 mM, 180 mM, 185 mM, 190 mM, 195 mM, 200 mM, 225 mM, 250 mM, 300 mM, 325 mM, 350 mM, 400 mM, 450 mM, or 500 mM. For example, the inventive vaccine composition may also comprise PBS as phosphate buffer, which comprises 137 mM NaCl, 2.7 mM KCl, 10 mM Na₂HPO₄ and 1.8 mM KH₂PO₄, or e.g. NaCl in a concentration of about 158 mM.

More specifically, the inventive vaccine composition is buffered by the buffer at a pH range of about pH 7-9, preferably of about pH 7.5 to about pH 8.8, or of about pH 7.8 to about pH 8.6, or of about pH 8.0 to about pH 8.4. Accordingly, the inventive vaccine composition is buffered by a buffer as disclosed above, e.g. by a Tris buffer, MOPS buffer, Na-acetate buffer, or phosphate buffer in concentrations as disclosed above. For example the inventive vaccine composition may be buffered at a pH range of about pH 7-9, e.g. of about pH 7.0, pH 7.1, pH 7.2, pH 7.3, pH 7.4, pH 7.5, pH 7.6, pH 7.7, pH 7.8, pH 7.9, pH 8.0 to about pH 8.4, pH 8.5, pH 8.6, pH 8.7, pH 8.8, pH 8.9, pH 9.0, or e.g. of about pH 7.8 to about pH 8.6, e.g. of about pH 7.8, pH 7.9, pH 8.0, pH 8.1, pH 8.2 to about pH 8.4, pH 8.5, pH 8.6, or at a pH range of about pH 8.0 to about pH 8.4, e.g. at about pH 8.0, pH 8.1, pH 8.2, pH 8.3, or pH 8.4. The pH of the buffer system as used above may be calculated according to any method known in the art, such as e.g. the Henderson-Haselbalch equation (pH=pKa+log₁₀([A⁻]/[HA]))

Moreover, the vaccine composition according to the invention may also comprise a preservative. The term “preservative” as used in the present invention shall mean any compound that when added to the inventive vaccine composition prolongs the time the inventive vaccine composition may be stored prior to use. Preservatives included with the inventive vaccine composition may include e.g. albumin, phenols, glycine, Thimerosal, benzalkonium chloride, polyaminopropyl biguanide, phenoxyethanol, merthiolate, gentamicin, neomycin, nystatin, amphotericin B, tetracycline, penicillin, streptomycin, polymyxin B, and any combination thereof. Accordingly, the inventive vaccine composition may comprise any of the above compounds in a concentration of about 0.001% (w/v)/(w/w) to about 5% (w/v)/(w/w), or of about 0.02% (w/v)/(w/w), 0.03% (w/v)/(w/w), 0.04% (w/v)/(w/w), 0.05% (w/v)/(w/w), 0.06% (w/v)/(w/w), 0.07% (w/v)/(w/w), 0.08% (w/v)/(w/w), 0.09% (w/v)/(w/w), 0.1% (w/v)/(w/w) to about 0.2% (w/v)/(w/w), 0.25% (w/v)/(w/w), 0.3% (w/v)/(w/w), 0.4% (w/v)/(w/w), 0.5% (w/v)/(w/w), 0.6% (w/v)/(w/w), 0.7% (w/v)/(w/w), 0.8% (w/v)/(w/w), 0.9% (w/v)/(w/w), 1.0% (w/v)/(w/w), 1.25% (w/v)/(w/w), 1.5% (w/v)/(w/w), 2.0% (w/v)/(w/w), 2.25% (w/v)/(w/w), 2.5% (w/v)/(w/w), 3% (w/v)/(w/w), 3.5% (w/v)/(w/w), 4% (w/v)/(w/w), 4.5% (w/v)/(w/w), 5% (w/v)/(w/w).

In a preferred embodiment, the inventive vaccine composition as disclosed above is for use in the vaccination of humans. The term “vaccination” as used in the context of the inventive vaccine composition refers to the administration of antigenic material, such as e.g. the inventive vaccine composition (a vaccine), to stimulate an individual's immune system to develop an adaptive immune response to a pathogen, such as HCMV in order to prevent, or reduce the risk of infection. Accordingly, the inventive vaccine or inventive vaccine composition will be administered to a human in a dose suitable to induce a sufficient immune response, e.g. an immune response that comprises T- and B-cell memory and neutralizing antibodies to provide protective immunity against a pathogen that comprises one or more proteins or protein complexes that comprise at least one, e.g. one, two, three, four or five, preferably five (5) of the amino acid sequences as disclosed above, e.g. UL128, UL130, UL131, gH and gL, or e.g. sequence variants thereof, such as e.g. SEQ ID NO:3, SEQ ID NO:31, SEQ ID NO:11, SEQ ID NO:21 and SEQ ID NO:25 or sequence variants thereof, or e.g. SEQ ID NO:3, SEQ ID NO:7, SEQ ID NO:33, SEQ ID NO:21 and SEQ ID NO:25 or sequence variants thereof, or e.g. SEQ ID NO:3, SEQ ID NO:7, SEQ ID NO:11, SEQ ID NO:35 and SEQ ID NO:25 or sequence variants thereof, or e.g. SEQ ID NO:3, SEQ ID NO:7, SEQ ID NO:11, SEQ ID NO:21 and SEQ ID NO:37 or sequence variants thereof, or e.g. SEQ ID NO:3, SEQ ID NO:31, SEQ ID NO:33, SEQ ID NO:21 and SEQ ID NO:25 or sequence variants thereof, or e.g. SEQ ID NO:3, SEQ ID NO:31, SEQ ID NO:33, SEQ ID NO:35 and SEQ ID NO:25 or sequence variants thereof, or e.g. SEQ ID NO:3, SEQ ID NO:31, SEQ ID NO:33, SEQ ID NO:35 and SEQ ID NO:37 or sequence variants thereof, or e.g. SEQ ID NO:3, SEQ ID NO:31, SEQ ID NO:33, SEQ ID NO:21 and SEQ ID NO:37 or sequence variants thereof, or e.g. SEQ ID NO:3, SEQ ID NO:31, SEQ ID NO:11, SEQ ID NO:35 and SEQ ID NO:37 or sequence variants thereof, or e.g. SEQ ID NO:3, SEQ ID NO:7, SEQ ID NO:33, SEQ ID NO:35 and SEQ ID NO:37, SEQ ID NO:3, SEQ ID NO:7, SEQ ID NO:11, SEQ ID NO:21 and SEQ ID NO:25 or sequence variants thereof.

In a fifth aspect the present invention provides a process for the preparation of a vaccine according to the disclosure as provided herein. Accordingly, the present invention provides for a process of the preparation of an inventive vaccine, which may e.g. comprise the steps of (i) using the inventive gene expression system as disclosed above or the stable cell line according to the present invention as described above for the expression of a soluble protein complex as disclosed above, (ii) purifying the inventive soluble protein complex obtainable by the inventive gene expression system or by a stable cell line according to the present invention as described above, and (iii) preparing a vaccine composition as disclosed above.

For example step (i) may include culturing the at least one mammalian cell as defined above, such as e.g. BHK, DUXB11, CHO-DG44, CHO-K1, CHO-K1SV, CHO-S, CHO-DXB11, CHO-K1SV GS knock-out (CHO-K1SV KO), CAP, PER.C6, NS0, Sp2/0, HEK293 T, HEK 293-F, HEK 6E, HEK293 EBNA, CAP-T, HELA, CVI, COS, R1610, BALBC/3T3, HAK, BFA-1c1BPT, RAJI, HT-1080, HKB-11, or preferably CHO-DG44, CHO-K1, CHO-K1SV, CHO-S, CHO-DXB11, CHO-K1SV GS knock-out (CHO-K1SV KO) cells, which have been transfected, or nucleofected with a vector comprising the nucleotide sequences as disclosed above, for the expression of the protein complex as defined above, which comprises the amino acid sequences according to e.g. SEQ ID NO:3, SEQ ID NO:31, SEQ ID NO:11, SEQ ID NO:21 and SEQ ID NO:25 or sequence variants thereof, or e.g. SEQ ID NO:3, SEQ ID NO:7, SEQ ID NO:33, SEQ ID NO:21 and SEQ ID NO:25 or sequence variants thereof, or e.g. SEQ ID NO:3, SEQ ID NO:7, SEQ ID NO:11, SEQ ID NO:35 and SEQ ID NO:25 or sequence variants thereof, or e.g. SEQ ID NO:3, SEQ ID NO:7, SEQ ID NO:11, SEQ ID NO:21 and SEQ ID NO:37 or sequence variants thereof, or e.g. SEQ ID NO:3, SEQ ID NO:31, SEQ ID NO:33, SEQ ID NO:21 and SEQ ID NO:25 or sequence variants thereof, or e.g. SEQ ID NO:3, SEQ ID NO:31, SEQ ID NO:33, SEQ ID NO:35 and SEQ ID NO:25 or sequence variants thereof, or e.g. SEQ ID NO:3, SEQ ID NO:31, SEQ ID NO:33, SEQ ID NO:35 and SEQ ID NO:37 or sequence variants thereof, or e.g. SEQ ID NO:3, SEQ ID NO:31, SEQ ID NO:33, SEQ ID NO:21 and SEQ ID NO:37 or sequence variants thereof, or e.g. SEQ ID NO:3, SEQ ID NO:31, SEQ ID NO:11, SEQ ID NO:35 and SEQ ID NO:37 or sequence variants thereof, or e.g. SEQ ID NO:3, SEQ ID NO:7, SEQ ID NO:33, SEQ ID NO:35 and SEQ ID NO:37, SEQ ID NO:3, SEQ ID NO:7, SEQ ID NO:11, SEQ ID NO:21 and SEQ ID NO:25 or sequence variants thereof, which are comprised in UL128, UL130, UL131, gH or gL or sequence variants thereof.

Accordingly, the purification step (ii) according to the present invention may e.g. employ affinity chromatography utilizing the Strep-tag technology, if at least one of the proteins of the inventive soluble protein complex comprises the amino acid sequence according to SEQ ID NO:17 and/or SEQ ID NO:39, or SEQ ID NO:17 and SEQ ID NO:39, or e.g. the purification step may require purification by means of Nickle-NTA agarose, if at least one of the proteins of the inventive soluble protein complex comprises the amino acid sequence according to SEQ ID NO:13 and SEQ ID NO:41 (6×His-tagged TEV), or SEQ ID NO:41 (6×His tag). Protocols for purification of soluble protein complexes are known in the art. For example, the purification of an inventive soluble protein complex as disclosed above may be done according to the method as described by Alsarraf et al, Acta Crystallogr Sect F Struct Biol Cryst Commun. Oct. 1, 2011; 67(Pt 10): 1253-1256, e.g. the cell culture medium may be incubated with e.g. 20 ml NTA agarose beads (Qiagen; pre-equilibrated with buffer A) for 1 h. The beads may then e.g. be washed with buffer B (50 mM Tris-HCl pH 8, 1 M NaCl, 50 mM imidazole, 5 mM β-mercaptoethanol and 1 mM benzamidine) and the protein may then e.g. be eluted with buffer E (50 mM Tris-HCl pH 8, 400 mM NaCl, 500 mM imidazole and 5 mM β-mercaptoethanol). The eluted protein may then e.g. be dialyzed in dialysis bags (cutoff e.g. 5 kDa) overnight at 277 K against 21 anion-exchange buffer (50 mM Tris-HCl pH 8 and 5 mM β-mercaptoethanol). Subsequently, the proteins may e.g. be spun down at 30 000 g for 10 min to remove protein aggregates. The supernatant may then e.g. be loaded onto a 2×5 ml Hi-Trap Q-FF anion-exchange column (GE Healthcare Life Sciences) equilibrated with anion-exchange buffer and the protein may be collected in the flowthrough (while the rest of the contaminants bound to the column). The inventive soluble protein complex may then be concentrated to 1 mg ml/ml and dialyzed against storage buffer (e.g. 50 mM Tris-HCl pH 7.6, 5 mM β-mercaptoethanol and 50% glycerol). For example, the inventive soluble protein complex comprising SEQ ID NO:13 or sequence variants thereof may also be further purified by treatment with TEV protease and e.g. subsequent dialysis as disclosed above, e.g. the inventive soluble protein complex comprising SEQ ID NO:13 or sequence variants thereof may be incubated with TEV protease e.g. for about 1 h, 2 h, 3 h, 4 h, 5 h, 6 h, or for about 6 h to about 12 h, and subsequently dialyzed or e.g. the inventive soluble protein complex as disclosed above, comprising SEQ ID NO:13 and SEQ ID NO:41 or sequence variants thereof, wherein the 6×His tag as according to amino acid sequence according to SEQ ID NO:41 or sequence variants thereof is located C-terminally, e.g. ENLYFQG-HHHHHH- and linked via a peptide bond to the TEV cleave site, may be purified in a first step as disclosed above, e.g. by a metal-affinity resin, such as e.g. Nickel-NTA, followed by subsequent incubation with TEV protease treatment and a further metal-affinity resin purification step to remove the cleaved TEV-6×His-tag fragments. The purified soluble protein complex may then e.g. be recovered from the flow-through.

More specifically, the present invention provides a process for preparing a vaccine composition, comprising the following steps:
(a) Preparation of a vector according to the present invention;
(b) Transfection of a mammalian producer cell with the vector prepared in step (a);
(c) Harvesting a HCMV pentamer comprising the amino acid sequences according to SEQ ID NO:3, SEQ ID NO:7, SEQ ID NO:11, SEQ ID NO:21 and SEQ ID NO:25 or sequence variants thereof from the mammalian producer cell;
(d) Optionally purification of the HCMV pentamer harvested in step (c); and
(e) Formulation of the harvested and optionally purified HCMV pentamer as a liquid or solid formulation.

It is understood that the HCMV pentamer harvested in step (c) is in particular the soluble protein complex according to the present invention as described above.

In step (a) a vector according to the present invention, e.g. a vector comprising the sequences as defined herein, is prepared for example by molecular cloning techniques known to the person skilled in the art.

In step (b) a mammalian producer cell, such as preferably BHK, DUXB11, CHO-DG44, CHO-K1, CHO-K1SV, CHO-S, CHO-DXB11, CHO-K1SV GS knock-out (CHO-K1SV KO), CAP, PER.C6, NS0, Sp2/0, HEK293 T, HEK 293-F, HEK 6E, HEK293 EBNA, CAP-T, HELA, CVI, COS, R1610, BALBC/3T3, HAK, BFA-1c1BPT, RAJI, HT-1080, HKB-11, or, more preferably, CHO-DG44, CHO-K1, CHO-K1SV, CHO-S, CHO-DXB11, CHO-K1SV GS knock-out (CHO-K1SV KO) cells, is transfected, preferably stably transfected, more preferably nucleofected, with the vector according to the present invention obtained in step (a). To this end for example the Lonza system may be used, e.g. by using the Nucleofector® Technology. For example, a cell-type specific Nucleofector® Kit may be used. Preferably, the transfection in step (b) of the process according to the present invention is thus a Nucleofection®. Particularly preferably, in the process according to the present invention the mammalian producer cell is a stable cell line according to the present invention as described herein.

Thereafter, the at least one mammalian cell transfected with the vector according to the present invention may preferably be seeded at a desired density depending e.g. on the cell line used, for example for CHO-K1SV e.g. 500000-2 million cells/ml, preferably 750000-1.5 million cells/ml, more preferably 800.000-1.2 million cells/ml, e.g. 1 million cells/ml, and cultured, e.g. for 1, 2, 3, 4, 5, 6, 7, 8, 9, 10, 11, 12, 13, 14, 15, 16, 17, 18, 19, 20, or more days.

Then, for example after 5-15 days, e.g. after 10 days, of culturing the transfected mammalian cells, the HCMV pentamer comprising the amino acid sequences according to SEQ ID NO:3, SEQ ID NO:7, SEQ ID NO:11, SEQ ID NO:21 and SEQ ID NO:25 or sequence variants thereof is harvested from the mammalian producer cell in step (c).

Preferably, in the process according to the present invention the HCMV pentamer is secreted by the mammalian producer cell and in step (c) the supernatant of the mammalian producer cell culture is harvested. Alternatively, in particular if the mammalian producer cells do not secrete the HCMV pentamer, the mammalian producer cells are harvested and disrupted, whereby cell disruption is a method or process for releasing biological molecules from inside a cell. Thereby the HCMV pentamer is released and can be harvested. However, to avoid the additional step of cell disruption, secretion of the HCMV pentamer from the producing mammalian cells is preferred.

In step (d), the HCMV pentamer harvested in step (c) is optionally purified. As described above, the purification step (d) according to the present invention may e.g. employ affinity chromatography utilizing the Strep-tag technology, if at least one of the proteins of the inventive soluble protein complex comprises the amino acid sequence according to SEQ ID NO:17 and/or SEQ ID NO:39, or SEQ ID NO:17 and SEQ ID NO:39, or e.g. the purification step may require purification by means of Nickle-NTA agarose, if at least one of the proteins of the inventive soluble protein complex comprises the amino acid sequence according to SEQ ID NO:13 and SEQ ID NO:41 (6×His-tagged TEV), or SEQ ID NO:41 (6×His tag). Protocols for purification of soluble protein complexes are known in the art. For example, the purification of an inventive soluble protein complex as disclosed above may be done according to the method as described by Alsarraf et al, Acta Crystallogr Sect F Struct Biol Cryst Commun. Oct. 1, 2011; 67(Pt 10): 1253-1256, e.g. the cell culture medium may be incubated with e.g. 20 ml NTA agarose beads (Qiagen; pre-equilibrated with buffer A) for 1 h. The beads may then e.g. be washed with buffer B (50 mM Tris-HCl pH 8, 1 M NaCl, 50 mM imidazole, 5 mM β-mercaptoethanol and 1 mM benzamidine) 5 and the protein may then e.g. be eluted with buffer E (50 mM Tris-HCl pH 8, 400 mM NaCl, 500 mM imidazole and 5 mM (3-mercaptoethanol). The eluted protein may then e.g. be dialyzed in dialysis bags (cutoff e.g. 5 kDa) overnight at 277 K against 21 anion-exchange buffer (50 mM Tris-HCl pH 8 and 5 mM (3-mercaptoethanol). Subsequently, the proteins may e.g. be spun down at 30 000 g for 10 min to remove protein aggregates. The supernatant may then e.g. be loaded onto a 2×5 ml Hi-Trap Q-FF anion-exchange column (GE Healthcare Life Sciences) equilibrated with anion-exchange buffer and the protein may be collected in the flowthrough (while the rest of the contaminants bound to the column). The inventive soluble protein complex may then be concentrated to 1 mg ml/ml and dialyzed against storage buffer (e.g. 50 mM Tris-HCl pH 7.6, 5 mM β-mercaptoethanol and 50% glycerol). For example, the inventive soluble protein complex comprising SEQ ID NO:13 or sequence variants thereof may also be further purified by treatment with TEV protease and e.g. subsequent dialysis as disclosed above, e.g. the inventive soluble protein complex comprising SEQ ID NO:13 or sequence variants thereof may be incubated with TEV protease e.g. for about 1 h, 2 h, 3 h, 4 h, 5 h, 6 h, or for about 6 h to about 12 h, and subsequently dialyzed or e.g. the inventive soluble protein complex as disclosed above, comprising SEQ ID NO:13 and SEQ ID NO:41 or sequence variants thereof, wherein the 6×His tag as according to amino acid sequence according to SEQ ID NO:41 or sequence variants thereof is located C-terminally, e.g. ENLYFQG-HHHHHH- and linked via a peptide bond to the TEV cleave site, may be purified in a first step as disclosed above, e.g. by a metal-affinity resin, such as e.g. Nickel-NTA, followed by subsequent incubation with TEV protease treatment and a further metal-affinity resin purification step to remove the cleaved TEV-6×His-tag fragments. The purified soluble) protein complex may then e.g. be recovered from the flow-through.

It is also preferred that the purification step (d) of the process according to the present invention comprises a substep (d1a) of affinity chromatography, preferably by using a tag-sequence comprised by the HCMV pentamer, e.g., if the HCMV pentamer comprises a Strep-tag, in substep (d1a) StrepTactin II chromatography may be performed.

Moreover, it is also preferred that the purification step (d) of the process according to the present invention comprises a substep (d2a), in particular following the substep (d1a), wherein a peptide cleavage site, which is preferably located in the HCMV pentamer between a C-terminus of a HCMV pentamer subunit, preferably UL131, and a tag-sequence, is cleaved. Preferably, a TEV cleavage site, e.g. according to SEQ ID NO:13, is located in the HCMV pentamer between a C-terminus of a HCMV pentamer subunit, preferably the C-terminus of UL131, and a tag-sequence, preferably a Strep-tag. Thereby it is preferred that cleavage is performed by treatment with TEV protease.

Alternatively, the purification step (d) according to the present invention may preferably comprise, in particular if the HCMV pentamer harvested in step (c) is a tagless version of the HCMV pentamer, tangential flow filtration, ion exchange chromatography, hydrophobic interaction chromatography, and/or size-exclusion chromatography.

Tangential flow filtration (TFF, also known as “crossflow filtration”, cf. http://en.wikipedia.org/wiki/Cross-flow_filtration) is a type of filtration (a particular unit operation), in which the majority of the feed flow travels tangentially across the surface of the filter, rather than into the filter. Preferably, tangential flow filtration is performed by using a filter membrane. TFF may preferably be a continuous process, unlike batch-wise dead-end filtration. Moreover, TFF may be improved by backwashing, clean-in-place systems, concentration, diafiltration and/or process flow disruption. TFF may serve to (i) concentrate the supernatant harvested in step (c), for example 2 fold-20 fold, preferably 5 fold-10 fold, and/or to efficiently remove small molecules. In particular, the filter membrane may be selected such that undesired gH/gL dimers or UL subunits are removed, whereas the desired pentamer remains; e.g. by using non-adsorbing membrane material or derivatives thereof with a 100 KDa cut off, for example polyethersulfone or regenerated cellulose or other derivatives of non-adsorbing membrane material with a 1 OOKDa cut off. Alternatively, also dead-end filtration may be used, however, TFF is preferred. In dead-end filtration the feed is passed through a membrane or bed, the solids being trapped in the filter and the filtrate being released at the other end.

Ion exchange chromatography (or ion chromatography; cf. http://en.wikipedia.org/wiki/lon_chromatography) is a process that allows the separation of ions and polar molecules based on their affinity to the ion exchanger. Ion exchange chromatography separates proteins with regards to their net charge, which is dependent on the composition of the mobile phase. By adjusting the pH or the ionic concentration of the mobile phase, various protein molecules can be separated. For example, if a protein has a net positive charge at pH 7, then it will bind to a column of negatively charged beads, whereas a negatively charged protein would not. By changing the pH so that the net charge on the protein is negative, it too will be eluted. Elution by increasing the ionic strength of the mobile phase is a more subtle effect—it works as ions from the mobile phase will interact with the immobilized ions in preference over those on the stationary phase. This “shields” the stationary phase from the protein, (and vice versa) and allows the protein to elute. Separation can be achieved based on the natural isoelectric point of the protein, which is preferred in the process according to the present invention. Thereby, the use of anion exchange, in particular anion-exchange chromatography, is particularly preferred. Alternatively a peptide tag can be genetically added to the protein to give the protein an isoelectric point away from most natural proteins (e.g. 6 arginines for binding to a cation-exchange resin or 6 glutamates for binding to an anion-exchange resin such as DEAE-Sepharose). Elution from ion-exchange columns can be sensitive to changes of a single charge-chromatofocusing. Ion-exchange chromatography allows purification of specific complexes according to both the number and the position of charged amino acids or charged peptide tags.

Hydrophobic interaction chromatography (cf. http://en.wikibooks.org/wiki/Proteomics/Protein_Separations_{—-—}Chromatography/Hydrophobicinteraction_Chromatography_%28HIC %29) is a separation technique that uses the properties of hydrophobicity to separate proteins from one another. In this type of chromatography, hydrophobic groups such as phenyl, octyl, or butyl, are attached to the stationary column. Proteins that pass through the column that have hydrophobic amino acid side chains on their surfaces are able to interact with and bind to the hydrophobic groups on the column. HIC separations are often designed using the opposite conditions of those used in ion exchange chromatography. In this separation, a buffer with a high ionic strength, usually ammonium sulfate, is initially applied to the column. The salt in the buffer reduces the solvation of sample solutes thus as solvation decreases, hydrophobic regions that become exposed are adsorbed by the medium. The more hydrophobic the molecule, the less salt needed to promote binding. To elute the proteins, the salt concentration is gradually decreased in order of increasing hydrophobicity. Additionally, elution can also be achieved through the use of mild organic modifiers or detergent. The stationary phase is designed to form hydrophobic interactions with other molecules. These interactions are too weak in water. However, addition of salts to the buffer result in hydrophobic interactions. The following is a list of salts that increase hydrophobic interactions in the order of their ability to enhance interactions:
1. Na₂SO₄
2. K₂SO₄
3. (NH₄)₂SO₄
4. NaCl
5. NH₄Cl
6. NaBr
7. NaSCN.

Thereby, the preferred salt in the context of the present invention is NaCl.

Although reversed phase chromatography and hydrophobic interaction chromatography are very similar, the ligands in reversed phase chromatography are much more hydrophobic than the ligands in hydrophobic interaction chromatography. This enables hydrophobic interaction chromatography to make use of more moderate elution conditions, which do not disrupt the sample nearly as much.

Size-exclusion chromatography (SEC; cf. http://en.wikipedia.org/wiki/Size-exclusion _chromatography) is a chromatographic method in which molecules in solution are separated by their size, and in some cases molecular weight. It is usually applied to large molecules or macromolecular complexes such as proteins and industrial polymers. Typically, when an aqueous solution is used to transport the sample through the column, the technique is known as gel-filtration chromatography, versus the name gel permeation chromatography, which is used when an organic solvent is used as a mobile phase. SEC is a widely used polymer characterization method because of its ability to provide good molar mass distribution (Mw) results for polymers. Size exclusion chromatography allows for both, separation from contamination as well as buffer exchange.

Preferably, in the process for preparing a vaccine composition according to the present invention, the purification step (d) comprises a substep (d1b) of tangential flow filtration, which is preferably followed by a substep (d2b) of ion exchange chromatography, hydrophobic interaction chromatography, and/or size-exclusion chromatography. More preferably, the substep (d2b) comprises ion exchange chromatography or hydrophobic interaction chromatography.

It is also preferred in the process for preparing a vaccine composition according to the present invention that the purification step (d) comprises a substep (d3b), wherein size exclusion chromatography is performed. Optionally, substep (d3b) follows substep (d1b) and/or substep (d2b). In other words, size exclusion chromatography may optionally be performed after a substep (d1b) of tangential flow filtration or after a substep (d2b) comprising e.g. ion exchange chromatography or hydrophobic interaction chromatography or, preferably, after a substep (d2b) comprising e.g. ion exchange chromatography or hydrophobic interaction chromatography, which was performed after a substep (d1b) of tangential flow filtration.

Thus, in a particularly preferred process for preparing a vaccine composition according to the present invention the purification step (d) comprises the following substeps:
(d1b) tangential flow filtration;
(d2b) ion exchange chromatography and/or hydrophobic interaction chromatography; and
(d3b) size-exclusion chromatography,
   whereby each of the substeps (d1b)-(d3b) may be performed once or repeatedly. If each of the substeps (d1b)-(d3b) is performed repeatedly, it is preferred that the above order of the substeps (d1b)-(d3b) is maintained, i.e. all repetitions of substep (d1b) are performed, thereafter all repetitions of substep (d2b) are performed, and thereafter all repetitions of substep (d3b) are performed.

Regarding size-exclusion chromatography, in particular as performed in substep (d3b), it is preferred that no further purification method, in particular no further chromatography method, is performed thereafter. In other words, size exclusion chromatography is preferably the last chromatography step, in particular the last chromatography step included in step (d).

In step (e) the harvested and optionally purified HCMV pentamer is formulated as a liquid or solid formulation to obtain a vaccine composition as described above.

Thus, in the process according to the present invention preferably (a) the vector according to the present invention is prepared, (b) a mammalian producer cell is transfected with the vector according to (a), (c) the soluble protein complex according to the present invention is harvested from the mammalian producer cell, (d) the complex harvested according to (c) is optionally purified, and (e) the harvested and optionally purified soluble complex is formulated as a liquid or solid formulation. Thereby, a vaccine composition is obtained.

Accordingly, the present invention also provides a vaccine composition obtainable by a process according to the present invention as described herein, which comprises optionally one or more additional pharmaceutically active components and, optionally, one or more pharmaceutically inactive components.

In sixth aspect, the present invention provides for a nucleic acid comprising SEQ ID NO:2, SEQ ID NO:4, SEQ ID NO:8, SEQ ID NO:12, SEQ ID NO:22, and SEQ ID NO:26 or sequence variants thereof, or SEQ ID NO:2, SEQ ID NO:4, SEQ ID NO:8, SEQ ID NO:12, SEQ ID NO:22, SEQ ID NO:26, SEQ ID NO:14, SEQ ID NO:16 and SEQ ID NO:42 or sequence variants thereof. Accordingly, the inventive nucleic acid may comprise the above sequences in any order, for as long as the nucleic acid can be used to transfect, or nucleofect mammalian cells as disclosed above, to obtain the inventive soluble protein complex.

In one embodiment, the inventive nucleic acid sequence further comprises SEQ ID NO:6 and/or SEQ ID NO:10 and/or SEQ ID NO:24, and/or SEQ ID NO:28, and/or SEQ ID NO:30, preferably comprising SEQ ID NO:24 and/or SEQ ID NO:28 and/or SEQ ID NO:30 or sequence variants thereof. Accordingly, the inventive nucleic acid may comprise e.g.SEQ ID NO:6 or SEQ ID NO:10 or SEQ ID NO:24, or SEQ ID NO:28, or SEQ ID NO:30 or sequence variants thereof, or e.g. SEQ ID NO:6 and SEQ ID NO:10 or sequence variants thereof, or e.g. SEQ ID NO:24, and SEQ ID NO:28, SEQ ID NO:30 and SEQ ID NO:6 or sequence variants thereof, or e.g. SEQ ID NO:30 and SEQ ID NO:10 or sequence variants thereof, or e.g. SEQ ID NO:30 and SEQ ID NO:24 or sequence variants thereof, or e.g. SEQ ID NO:30 and SEQ ID NO:28 or sequence variants thereof, or e.g. or SEQ ID NO:10 or SEQ ID NO:24 or sequence variants thereof, or e.g. or SEQ ID NO:10 or SEQ ID NO:28 or sequence variants thereof, preferably the inventive nucleic acid comprises SEQ ID NO:24 and/or SEQ ID NO:28 and/or SEQ ID NO:30 or sequence variants thereof, e.g. SEQ ID NO:24 or SEQ ID NO:28 or SEQ ID NO:30 or sequence variants thereof, or e.g. SEQ ID NO:24 and SEQ ID NO:28 or sequence variants thereof, or e.g. SEQ ID NO:24 and SEQ ID NO:30 or sequence variants thereof, or e.g. SEQ ID NO:28 and SEQ ID NO:30 or sequence variants thereof, or e.g. SEQ ID NO:24 and SEQ ID NO:28 and SEQ ID NO:30 or sequence variants thereof.

More specifically, the inventive nucleic acid may comprise operably linked in 5′ to 3′ direction the nucleotide sequences according to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 and SEQ ID NO:26 or sequence variants thereof. The term “operably linked” as used with the inventive nucleic acid refers to nucleic acid which are juxtaposed in such a way that their respective functions are mutually dependent. For example, a promoter operably linked to a coding sequence is capable of effecting the expression of the coding sequence. The term “operably linked” may also be independent of the location a respective sequence, as long as the functional interrelationship between the two sequences is maintained, e.g. the nucleotide sequences as disclosed above may not be adjacent next to each other in 5′-3′ direction, but may e.g. be separated by nucleotide sequences of undefined length.

According to one embodiment, the inventive nucleic acid comprises the above nucleotide sequences in any given order operably linked in 5′ to 3′ direction, for as long as the inventive nucleotide sequence encodes the soluble protein complex according to the invention, e.g. the inventive nucleic acid comprises operably linked in 5′ to 3′ direction the nucleotide sequences according to SEQ ID NO:2, SEQ ID NO:4, SEQ ID NO:10, SEQ ID NO:8, SEQ ID NO:6, SEQ ID NO:12, SEQ ID NO:14, SEQ ID NO:16, SEQ ID NO:18, SEQ ID NO:20, SEQ ID NO:26, SEQ ID NO:24 and SEQ ID NO:22 or sequence variants thereof, or e.g. SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 and SEQ ID NO:26 or sequence variants thereof, or e.g. SEQ ID NO:20, SEQ ID NO:22, SEQ ID NO:24, SEQ ID NO:26, SEQ ID NO:24, SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18 or sequence variants thereof, or e.g. SEQ ID NO:20, SEQ ID NO:22, SEQ ID NO:10, SEQ ID NO:26, SEQ ID NO:10, SEQ ID NO:2, SEQ ID NO:4, SEQ ID NO:10, SEQ ID NO:8, SEQ ID NO:6, SEQ ID NO:12, SEQ ID NO:14, SEQ ID NO:16, SEQ ID NO:18 or sequence variants thereof, or e.g. SEQ ID NO:20, SEQ ID NO:22, SEQ ID NO:10, SEQ ID NO:26, SEQ ID NO:2, SEQ ID NO:4, SEQ ID NO:10, SEQ ID NO:8, SEQ ID NO:6, SEQ ID NO:12, SEQ ID NO:14, SEQ ID NO:16, SEQ ID NO:18 or sequence variants thereof.

According to one embodiment, the inventive nucleic acid comprises operably linked in 5′ to 3′ direction the nucleotide sequences according to SEQ ID NO:20, SEQ ID NO:4, SEQ ID NO:28, SEQ ID NO:32, SEQ ID NO:28, SEQ ID NO:34, SEQ ID NO:20, SEQ ID NO:36, SEQ ID NO:28 and SEQ ID NO:38 or sequence variants thereof. Accordingly, the inventive nucleic acid comprises the above nucleotide sequences in any given order operably linked in 5′ to 3′ direction, for as long as the inventive nucleic acid encodes the soluble protein complex according to the invention, e.g. the inventive nucleic acid comprises operably linked in 5′ to 3′ direction the nucleotide sequences according to SEQ ID NO:20, SEQ ID NO:36, SEQ ID NO:28 and SEQ ID NO:38, SEQ ID NO:20, SEQ ID NO:4, SEQ ID NO:28, SEQ ID NO:32, SEQ ID NO:28, SEQ ID NO:34 or sequence variants thereof, or e.g. SEQ ID NO:20, SEQ ID NO:38, SEQ ID NO:28 and SEQ ID NO:36, SEQ ID NO:20, SEQ ID NO:4, SEQ ID NO:28, SEQ ID NO:32, SEQ ID NO:28, SEQ ID NO:34 or sequence variants thereof, or.e.g. SEQ ID NO:20, SEQ ID NO:38, SEQ ID NO:28 and SEQ ID NO:36, SEQ ID NO:20, SEQ ID NO:4, SEQ ID NO:28, SEQ ID NO:34, SEQ ID NO:28, SEQ ID NO:32 or sequence variants thereof.

More specifically, the inventive nucleic acid may comprise operably linked in 5′ to 3′ direction the nucleotide sequences according to SEQ ID NO:20, SEQ ID NO:4, SEQ ID NO:30, SEQ ID NO:32, SEQ ID NO:30, SEQ ID NO:34, SEQ ID NO:20, SEQ ID NO:36, SEQ ID NO:30 and SEQ ID NO:38 or sequence variants thereof, or e.g. SEQ ID NO:20, SEQ ID NO:36, SEQ ID NO:30 SEQ ID NO:38, SEQ ID NO:20, SEQ ID NO:4, SEQ ID NO:30, SEQ ID NO:32, SEQ ID NO:30, SEQ ID NO:34 or sequence variants thereof, or e.g.SEQ ID NO:20, SEQ ID NO:38, SEQ ID NO:30 SEQ ID NO:36, SEQ ID NO:20, SEQ ID NO:4, SEQ ID NO:30, SEQ ID NO:32, SEQ ID NO:30, SEQ ID NO:34 or sequence variants thereof, or e.g. SEQ ID NO:20, SEQ ID NO:32, SEQ ID NO:30, SEQ ID NO:4, SEQ ID NO:30, SEQ ID NO:34, SEQ ID NO:20, SEQ ID NO:36, SEQ ID NO:30 and SEQ ID NO:38 or sequence variants thereof, or e.g. SEQ ID NO:20, SEQ ID NO:4, SEQ ID NO:30, SEQ ID NO:34, SEQ ID NO:30, SEQ ID NO:32, SEQ ID NO:20, SEQ ID NO:36, SEQ ID NO:30 and SEQ ID NO:38 or sequence variants thereof, or e.g. SEQ ID NO:20, SEQ ID NO:34, SEQ ID NO:30, SEQ ID NO:32, SEQ ID NO:30, SEQ ID NO:4, SEQ ID NO:20, SEQ ID NO:36, SEQ ID NO:30 and SEQ ID NO:38 or sequence variants thereof.

More specifically, the inventive nucleic acid may comprise operably linked in 5′ to 3′ direction SEQ ID NO:20, SEQ ID NO:4, SEQ ID NO:28, SEQ ID NO:32, SEQ ID NO:28, SEQ ID NO:34, SEQ ID NO:14, SEQ ID NO:16, SEQ ID NO:40, SEQ ID NO:42, SEQ ID NO:20, SEQ ID NO:36, SEQ ID NO:28 and SEQ ID NO:38 or sequence variants thereof, or e.g. SEQ ID NO:20, SEQ ID NO:36, SEQ ID NO:28, SEQ ID NO:38, SEQ ID NO:20, SEQ ID NO:4, SEQ ID NO:28, SEQ ID NO:32, SEQ ID NO:28, SEQ ID NO:34, SEQ ID NO:14, SEQ ID NO:16, SEQ ID NO:40, SEQ ID NO:42 or sequence variants thereof, or e.g. SEQ ID NO:20, SEQ ID NO:32, SEQ ID NO:28, SEQ ID NO:4, SEQ ID NO:28, SEQ ID NO:34, SEQ ID NO:14, SEQ ID NO:16, SEQ ID NO:40, SEQ ID NO:42, SEQ ID NO:20, SEQ ID NO:36, SEQ ID NO:28 and SEQ ID NO:38 or sequence variants thereof, or e.g. SEQ ID NO:20, SEQ ID NO:32, SEQ ID NO:28, SEQ ID NO:34, SEQ ID NO:28, SEQ ID NO:4, SEQ ID NO:14, SEQ ID NO:16, SEQ ID NO:40, SEQ ID NO:42, SEQ ID NO:20, SEQ ID NO:36, SEQ ID NO:28 and SEQ ID NO:38 or sequence variants thereof, or e.g. SEQ ID NO:20, SEQ ID NO:38, SEQ ID NO:28, SEQ ID NO:36, SEQ ID NO:20, SEQ ID NO:4, SEQ ID NO:28, SEQ ID NO:32, SEQ ID NO:28, SEQ ID NO:34, SEQ ID NO:14, SEQ ID NO:16, SEQ ID NO:40, SEQ ID NO:42 or sequence variants thereof, or e.g.SEQ ID NO:20, SEQ ID NO:32, SEQ ID NO:28, SEQ ID NO:4, SEQ ID NO:28, SEQ ID NO:34, SEQ ID NO:14, SEQ ID NO:16, SEQ ID NO:40, SEQ ID NO:42, SEQ ID NO:20, SEQ ID NO:38, SEQ ID NO:28 and SEQ ID NO:36 or sequence variants thereof, or e.g. SEQ ID NO:20, SEQ ID NO:32, SEQ ID NO:28, SEQ ID NO:34, SEQ ID NO:28, SEQ ID NO:4, SEQ ID NO:14, SEQ ID NO:16, SEQ ID NO:40, SEQ ID NO:42, SEQ ID NO:20, SEQ ID NO:38, SEQ ID NO:28 and SEQ ID NO:36 or sequence variants thereof.

More specifically, the inventive nucleic acid may comprise operably linked in 5′ to 3′ direction SEQ ID NO:20, SEQ ID NO:4, SEQ ID NO:30, SEQ ID NO:32, SEQ ID NO:30, SEQ ID NO:34, SEQ ID NO:14, SEQ ID NO:16, SEQ ID NO:40, SEQ ID NO:42, SEQ ID NO:20, SEQ ID NO:36, SEQ ID NO:30 and SEQ ID NO:38, or e.g. SEQ ID NO:20, SEQ ID NO:36, SEQ ID NO:30, SEQ 10 ID NO:38, SEQ ID NO:20, SEQ ID NO:4, SEQ ID NO:30, SEQ ID NO:32, SEQ ID NO:30, SEQ ID NO:34, SEQ ID NO:14, SEQ ID NO:16, SEQ ID NO:40, SEQ ID NO:42, or e.g. SEQ ID NO:20, SEQ ID NO:38, SEQ ID NO:30, SEQ ID NO:36, SEQ ID NO:20, SEQ ID NO:4, SEQ ID NO:30, SEQ ID NO:32, SEQ ID NO:30, SEQ ID NO:34, SEQ ID NO:14, SEQ ID NO:16, SEQ ID NO:40, SEQ ID NO:42, or e.g. SEQ ID NO:20, SEQ ID NO:36, SEQ ID NO:30, SEQ ID NO:38, SEQ ID NO:20, SEQ ID NO:32, SEQ ID NO:30, SEQ ID NO:4, SEQ ID NO:30, SEQ ID NO:34, SEQ ID NO:14, SEQ ID NO:16, SEQ ID NO:40, SEQ ID NO:42, or e.g. SEQ ID NO:20, SEQ ID NO:36, SEQ ID NO:30, SEQ ID NO:38, SEQ ID NO:20, SEQ ID NO:4, SEQ ID NO:30, SEQ ID NO:34, SEQ ID NO:30, SEQ ID NO:32, SEQ ID NO:14, SEQ ID NO:16, SEQ ID NO:40, SEQ ID NO:42, or e.g. SEQ ID NO:20, SEQ ID NO:36, SEQ ID NO:30, SEQ ID NO:38, SEQ ID NO:20, SEQ ID NO:32, SEQ ID NO:30, SEQ ID NO:34, SEQ ID NO:30, SEQ ID NO:4, SEQ ID NO:14, SEQ ID NO:16, SEQ ID NO:40, SEQ ID NO:42, or e.g. SEQ ID NO:20, SEQ ID NO:38, SEQ ID NO:30, SEQ ID NO:36, SEQ ID NO:20, SEQ ID NO:32, SEQ ID NO:30, SEQ ID NO:4, SEQ ID NO:30, SEQ ID NO:34, SEQ ID NO:14, SEQ ID NO:16, SEQ ID NO:40, SEQ ID NO:42, or e.g. SEQ ID NO:20, SEQ ID NO:38, SEQ ID NO:30, SEQ ID NO:36, SEQ ID NO:20, SEQ ID NO:4, SEQ ID NO:30, SEQ ID NO:34, SEQ ID NO:30, SEQ ID NO:32, SEQ ID NO:14, SEQ ID NO:16, SEQ ID NO:40, SEQ ID NO:42, or e.g. SEQ ID NO:20, SEQ ID NO:38, SEQ ID NO:30, SEQ ID NO:36, SEQ ID NO:20, SEQ ID NO:32, SEQ ID NO:30, SEQ ID NO:34, SEQ ID NO:30, SEQ ID NO:4, SEQ ID NO:14, SEQ ID NO:16, SEQ ID NO:40, SEQ ID NO:42.

More specifically, the inventive nucleic acid comprises operably linked in 5′ to 3′ direction SEQ ID NO:20, SEQ ID NO:4, SEQ ID NO:28, SEQ ID NO:32, SEQ ID NO:28, SEQ ID NO:34, SEQ ID NO:14, SEQ ID NO:16, SEQ ID NO:40, SEQ ID NO:20, SEQ ID NO:36, SEQ ID NO:28 and SEQ ID NO:38 or sequence variants thereof, or e.g. SEQ ID NO:20, SEQ ID NO:36, SEQ ID NO:28, SEQ ID NO:38, SEQ ID NO:20, SEQ ID NO:4, SEQ ID NO:28, SEQ ID NO:32, SEQ ID NO:28, SEQ ID NO:34, SEQ ID NO:14, SEQ ID NO:16, SEQ ID NO:40 or sequence variants thereof, or e.g. SEQ ID NO:20, SEQ ID NO:38, SEQ ID NO:28, SEQ ID NO:36, SEQ ID NO:20, SEQ ID NO:4, SEQ ID NO:28, SEQ ID NO:32, SEQ ID NO:28, SEQ ID NO:34, SEQ ID NO:14, SEQ ID NO:16, SEQ ID NO:40 or sequence variants thereof, or e.g.SEQ ID NO:20, SEQ ID NO:32, SEQ ID NO:28, SEQ ID NO:4, SEQ ID NO:28, SEQ ID NO:34, SEQ ID NO:14, SEQ ID NO:16, SEQ ID NO:40, SEQ ID NO:20, SEQ ID NO:36, SEQ ID NO:28 and SEQ ID NO:38 or 0.5 sequence variants thereof, or e.g. SEQ ID NO:20, SEQ ID NO:34, SEQ ID NO:28, SEQ ID NO:32, SEQ ID NO:28, SEQ ID NO:4, SEQ ID NO:14, SEQ ID NO:16, SEQ ID NO:40, SEQ ID NO:20, SEQ ID NO:36, SEQ ID NO:28 and SEQ ID NO:38 or sequence variants thereof, or e.g. SEQ ID NO:20, SEQ ID NO:4, SEQ ID NO:28, SEQ ID NO:34, SEQ ID NO:28, SEQ ID NO:32, SEQ ID NO:14, SEQ ID NO:16, SEQ ID NO:40, SEQ ID NO:20, SEQ ID NO:36, SEQ ID NO:28 and SEQ ID NO:38 or sequence variants thereof, or e.g. SEQ ID NO:20, SEQ ID NO:32, SEQ ID NO:28, SEQ ID NO:4, SEQ ID NO:28, SEQ ID NO:34, SEQ ID NO:14, SEQ ID NO:16, SEQ ID NO:40, SEQ ID NO:20, SEQ ID NO:38, SEQ ID NO:28 and SEQ ID NO:36 or sequence variants thereof, or e.g. SEQ ID NO:20, SEQ ID NO:34, SEQ ID NO:28, SEQ ID NO:32, SEQ ID NO:28, SEQ ID NO:4, SEQ ID NO:14, SEQ ID NO:16, SEQ ID NO:40, SEQ ID NO:20, SEQ ID NO:38, SEQ ID NO:28 and SEQ ID NO:36 or sequence variants thereof, or e.g. SEQ ID NO:20, SEQ ID NO:4, SEQ ID NO:28, SEQ ID NO:34, SEQ ID NO:28, SEQ ID NO:32, SEQ ID NO:14, SEQ ID NO:16, SEQ ID NO:40, SEQ ID NO:20, SEQ ID NO:38, SEQ ID NO:28 and SEQ ID NO:36 or sequence variants thereof.

More specifically, the inventive nucleic acid may comprise operably linked in 5′ to 3′ direction SEQ ID NO:20, SEQ ID NO:4, SEQ ID NO:30, SEQ ID NO:32, SEQ ID NO:30, SEQ ID NO:34, SEQ ID NO:14, SEQ ID NO:16, SEQ ID NO:40, SEQ ID NO:20, SEQ ID NO:36, SEQ ID NO:30 and SEQ ID NO:38 or sequence variants thereof, or e.g. SEQ ID NO:20, SEQ ID NO:38, SEQ ID NO:30 and SEQ ID NO:36, SEQ ID NO:20, SEQ ID NO:4, SEQ ID NO:30, SEQ ID NO:32, SEQ ID NO:30, SEQ ID NO:34, SEQ ID NO:14, SEQ ID NO:16, SEQ ID NO:40 or sequence variants thereof, or e.g. SEQ ID NO:20, SEQ ID NO:32, SEQ ID NO:30, SEQ ID NO:4, SEQ ID NO:30, SEQ ID NO:34, SEQ ID NO:14, SEQ ID NO:16, SEQ ID NO:40, SEQ ID NO:20, SEQ ID NO:36, SEQ ID NO:30 and SEQ ID NO:38 or sequence variants thereof, or e.g. SEQ ID NO:20, SEQ ID NO:32, SEQ ID NO:30, SEQ ID NO:34, SEQ ID NO:30, SEQ ID NO:4, SEQ ID NO:14, SEQ ID NO:16, SEQ ID NO:40, SEQ ID NO:20, SEQ ID NO:36, SEQ ID NO:30 and SEQ ID NO:38 or sequence variants thereof, or e.g. SEQ ID NO:20, SEQ ID NO:34, SEQ ID NO:30, SEQ ID NO:32, SEQ ID NO:30, SEQ ID NO:4, SEQ ID NO:14, SEQ ID NO:16, SEQ ID NO:40, SEQ ID NO:20, SEQ ID NO:36, SEQ ID NO:30 and SEQ ID NO:38 or sequence variants thereof, or e.g. SEQ ID NO:20, SEQ ID NO:36, SEQ ID NO:30, SEQ ID NO:38, SEQ ID NO:20, SEQ ID NO:32, SEQ ID NO:30, SEQ ID NO:4, SEQ ID NO:30, SEQ ID NO:34, SEQ ID NO:14, SEQ ID NO:16, SEQ ID NO:40 or sequence variants thereof, or e.g. SEQ ID NO:20, SEQ ID NO:36, SEQ ID NO:30, SEQ ID NO:38, SEQ ID NO:20, SEQ ID NO:32, SEQ ID NO:30, SEQ ID NO:34, SEQ ID NO:30, SEQ ID NO:4, SEQ ID NO:14, SEQ ID NO:16, SEQ ID NO:40 or sequence variants thereof, or e.g. SEQ ID NO:20, SEQ ID NO:36, SEQ ID NO:30, SEQ ID NO:38, SEQ ID NO:20, SEQ ID NO:34, SEQ ID NO:30, SEQ ID NO:32, SEQ ID NO:30, SEQ ID NO:4, SEQ ID NO:14, SEQ ID NO:16, SEQ ID NO:40 or sequence variants thereof.

In one embodiment, the inventive nucleic acid comprises the nucleotide sequence according to SEQ ID NO:44, or SEQ ID NO:46, or SEQ ID NO:48, or SEQ ID NO:50 or sequence variants thereof. For example, the inventive nucleic acid comprising the nucleotide sequence according to SEQ ID NO:44 or sequence variants thereof encodes the amino acid sequence of the inventive soluble protein complex comprising the amino acid sequence according to SEQ ID NO:43 or sequence variants thereof, or e.g. the inventive nucleic acid comprising the nucleotide sequence according to SEQ ID NO:46 or sequence variants thereof encodes the amino acid sequence of the inventive soluble protein complex comprising the amino acid sequence according to SEQ ID NO:45 or sequence variants thereof, or e.g. the inventive nucleic acid comprising the nucleotide sequence according to SEQ ID NO:48 or sequence variants thereof encodes the amino acid sequence of the inventive soluble protein complex comprising the amino acid sequence according to SEQ ID NO:47 or sequence variants thereof, or e.g. the inventive nucleic acid comprising the nucleotide sequence according to SEQ ID NO:50 or sequence variants thereof encodes the amino acid sequence of the inventive soluble protein complex comprising the amino acid sequence according to SEQ ID NO:49 or sequence variants thereof.

In one embodiment, the invention provides for a nucleic acid as disclosed above for use in a process according to any one of the above embodiments, e.g. for use in the inventive gene expression system, or e.g. to obtain the inventive soluble protein complex as disclosed above, or e.g. in a process to obtain the inventive vaccine composition as disclosed above.

In a seventh aspect, the present invention provides for a mammalian cell comprising at least one nucleic acid according to the present invention for use in a process according to the present invention.

In a more specific embodiment, the present invention provides for a CHO cell, which comprises at least one inventive nucleic acid as disclosed above for use in a process for the preparation of a vaccine according to the invention. The term “CHO cell” as used in the above embodiment of the present invention refers to any cell selected from CHO-DG44, CHO-K1, CHO-K1SV, CHO-S, CHO-DX811, or CHO-K1SV GS knock-out (CHO-K1SV KO) cell types. The term CHO cell as used also includes at least one CHO cell as disclosed above, e.g. the term CHO cell refers to at least 1, or at least 10, or at least 100, or at least 1000, or at least about 10,000 cells, or of at least about 10⁵, 10⁶, 10⁷, 10⁸, 10⁹, 10¹⁰, 10¹¹, 10¹² CHO cells as disclosed above, or e.g. if the CHO cells are grown in a non-adherent culture of about 10³ cells/ml, or of about 10⁴ cells/ml, to about 10⁹ cells/ml, e.g. 10⁵ cells/ml, 10⁶ cells/ml, 10⁷ cells/ml, 10⁸ cells/ml, or of about 2.5×10² cells/ml, 3×10² cells/ml, 5×10² cells/ml, 10³ cells/ml, 1.25×10³ cells/ml, 2.5×10³ cells/ml, 5×10³ cells/ml, 7.5×10³ cells/ml, 1×10⁴ cells/ml, 2.5×10⁴ cells/ml, 5×10⁴ cells/ml, 7.5×10⁴ cells/ml, 1×10⁵ cell/ml to about 2.5×10⁵ ells/ml, 5×10⁵ cells/ml, 7.5×10⁵ cells/ml, 1×10⁶ cells/ml, 2.5×10⁶ cells/ml, 5×10⁶ cells/ml, 7.5×10⁶ cells/ml, 1×10⁷ cells/ml, 5×10⁸ cells/ml, 1×10⁸ cells/ml, 2.5×10⁸ cells/ml, 5×10⁸ cells/ml, 1×10⁹ cells/ml. The CHO cell comprising at least one nucleic acid according to the present invention may e.g. comprise 1, 2, 3, 4, 5, 6, 7, 8, 9, 10, 10², 10³, 10⁴ nucleic acids according to the invention, or e.g. of about 1, 2, 3, 4, 5, 6, 7, 8, 9, 10 inventive nucleic acid molecules to about 10², 10³ inventive nucleic acid molecules, e.g. expression vectors. The expression vector may be any of e.g. a viral vector selected from the group consisting of a plasmid or an adeno-associated virus, a retrovirus, a vaccinia virus, an oncolytic adenovirus, and the like, or e.g. a as comprised in the inventive gene expression system, e.g. such as those disclosed in the appended examples.

According to an eight aspect, the present invention provides for a kit of parts, which comprises the inventive gene expression system as disclosed above. Accordingly, the present invention provides for, or relates to a kit, such as a kit of parts, that includes a plurality of components for the construction and/or use of the inventive gene expression system. For example, the kit of parts according to the invention may comprise at least two components that include (preferably separately): (i) a vector comprising the inventive transcription system, and (ii) at least one other component for the use of the inventive gene expression system, such as e.g. at least one mammalian cell, e.g. preferably at least one CHO cell as defined above and (iii) optionally reagents, such as e.g. reagents for the transfection of the at least one mammalian cell comprised in the kit with the inventive nucleic acid. Such reagents may include e.g. liposomal transfection agents, or non-liposomal transfection agents, such as FuGene® or Lipofectamine 2000® transfection reagents. The vector comprised in the inventive kit of parts may e.g. be provided as an ethanolic precipitate, lyophilized and may be provided in an amount e.g. about 1 μg to about 100 μg, or e.g. in an amount of e.g. 10 μg to about 50 μg, or in an amount of about 25 μg to about 75 μg, e.g. in an amount of about 15 μg, of about 20 μg, of about 25 μg, of about 30 μg, of about 35 μg, of about 40 μg, of about 50 μg, of about 60 μg, of about 70 μg, of about 80 μg or of about 90 μg. The inventive kit of parts may e.g. also comprise as second (ii) component at least one mammalian cell as defined above, such as e.g. CHO cells as defined above, which have been transfected with the inventive nucleic acid. The at least one mammalian cell may e.g. also be provided in a suitable culture medium, such as e.g. Freestyle® CHO expression medium, or ProCHO™ medium, or PowerCHO™, or UltraCHO™, or any other culture medium suited for the expression of the HCMV surface glycoproteins according to the invention. The culture medium may, however, also form a separate part of the inventive kit of parts.

The plurality of components in the inventive kit may be presented, packaged or stored separately. For example, the components of the inventive kit of parts may be isolated from one another by being held in separate containers, e.g. such components, although held separately, may be boxed or otherwise associated together to aid storage and/or transport, and such association may include additional components. The term “transfection” as used with the inventive kit, or with the present invention, refers to the uptake of foreign DNA by a cell, e.g. by the at least one mammalian cell as disclosed above. Accordingly, a cell has been “transfected” when exogenous DNA, such as any one of the inventive nucleic acids as disclosed above, has been introduced into a cell. A number of transfection techniques are generally known in the art, see, e.g., Graham et al. (1973) Virology, 52:456, or Green et al. “Molecular Cloning—a laboratory manual” CSH Laboratory Press, 2012, or Davis et al. (1986) Basic Methods in Molecular Biology, Elsevier, and Chu et al. (1981) Gene 13:197. Such techniques can be used to introduce one or more exogenous DNA moieties into suitable host cells. The term refers to both stable and transient uptake of the genetic material e.g. of the inventive nucleic acid, and includes uptake of peptide- or antibody-linked DNAs. The inventive vector comprising a transcription system as disclosed above refers to an assembly which is capable of directing the expression of a one or more sequences or genes of interest. The inventive nucleic acid expression vector includes one or more promoters, e.g. two, three, four or more promoters, which are operably linked to the nucleotide sequences according to the invention and optionally to additional gene(s) of interest. For example, other control elements may be present on the vector as well. The inventive vector as disclosed above may e.g. also comprise and in addition to the components of the transcription system, a bacterial origin of replication, and e.g. one or more selectable markers, such as e.g. blasticidin resistance, G-418 resistance, hygromycin B resistance, puromycin resistance, zeocin resistance, or e.g. ampicillin resistance and/or kanamycin resistance genes. The vector may further comprise e.g. a signal which allows the plasmid construct to exist as single-stranded DNA (e.g., a MI 3 origin of replication), a multiple cloning site, and a “mammalian” origin of replication (e.g., a SV40 or adenovirus origin of replication).

In a ninth aspect, the present invention relates to a method of vaccinating a human, wherein the method comprises administering to a person the inventive vaccine composition as disclosed above in therapeutically effective amounts. Accordingly, the present invention relates to a method of administering to a person a therapeutically effective amount of the inventive vaccine composition as disclosed above. The term “therapeutically effective amount” as used herein means an amount of the inventive vaccine composition administered which is of sufficient quantity to achieve the intended purpose, e.g. to induced a protective immune response, involving e.g. both innate and adaptive immune responses. For example, the inventive method of vaccinating a human may comprise providing the inventive vaccine or vaccine composition as disclosed above to a human, e.g. the inventive vaccine or vaccine composition as disclosed above may be administered orally (p.o.), or e.g. intravenously (i.v.), or intra muscular (i.m.), or e.g. transdermally, or e.g. via inhalation, or e.g. subcutaneously, e.g. by injection or by a particle delivery system, such as a gene gun. Herein, the vaccine may e.g. be comprised in or on the particles delivered by the gene gun.

More specifically, the inventive method of vaccination may comprise administering to a human about 0.2 to about 200 μg, or about 2 μg to about 150 μg, or about 5 μg- to about 100 μg, or about 10 μg to about 90 μg, or about 15 to about 80 μg of the vaccine composition according to the invention as disclosed above. Accordingly, the inventive method of vaccination comprises administering to a human about 0.2 μg to about 200 μg of the inventive vaccine composition, e.g. about 0.5 μg to about 195 μg, or e.g. about 1 μg to about 190 μg, or e.g. about 1.5 μg to about 185 μg, or e.g. about 2 μg to about 180 μg, or e.g. about 2.5 μg to about 175 μg, or e.g. about 5 μg to about 170 μg, or e.g. about 10 μg to about 160 μg, or e.g. 15 μg to about 150 μg, or e.g. 20 μg to about 145 μg, or e.g. 25 μg to about 140 μg, or e.g. about 30 μg to about 130 μg, or e.g. about 35 μg to about 125 μg, or e.g. about 40 μg to about 120 μg, or e.g. about 45 μg to about 115 μg, or e.g. about 50 μg to about 110 μg, or e.g. about 55 μg to about 100 μg, or e.g. about 60 μg to about 95 μg, or e.g. 65 μg to about 90 μg, or e.g. about 70 μg to about 85 μg, or e.g. about 75 μg to about 80 μg, or e.g., or e.g. about 2.0 μg, 2.5 μg, 3.0 μg, 3.5 μg, 4 g, 4.5 g, 5 μg, 5.5 μg, 6 μg, 6.5 μg, 7 μg, 7.5 μg, 8 μg, 8.5 μg, 9 μg, 9.5 μg, 10 μg, 10.5 μg, 11 g, 11.5 μg, 12 μg, 12.5 μg, 13 μg, 13.5 μg, 14 μg, 14.5 μg, 15 μg, 15.5 μg, 16 μg, 16.5 μg, 17 μg, 17.5 μg, 18 μg, 18.5 μg, 19 μg 19.5 μg, 20 μg to about 25 g, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 47.5 μg, 50 μg, 52.5 μg, 55 μg, 57.5 μg, 60 μg, 62.5 μg, 65 μg, 67.5 μg, 70 μg, 72.5 μg, 75 μg, 77.5 μg, 80 μg, 82.5 μg, 85 μg, 87.5 μg, 90 μg, 92.5 μg, 95 97.5 μg, 100 μg to e.g. about 105 μg, 107.5 μg, 110 μg, 112.5 μg, 115 μg, 117.5 μg, 120 μg, 122.5 μg, 125 μg, 127.5 μg, 130 μg, 135 μg, 140 μg, 150 μg, 155 μg, 160 μg, 165 μg, 170 μg, 175 μg, 180 μg, 185 μg, 190 μg, 195 μg, or 200 μg, or e.g. about 0.5 μg, 1 μg, 2.0 μg, 2.5 μg, 3.0 μg, 3.5 μg, 4 μg, 4.5 μg, 5 μg, 5.5 μg, 6 μg, 6.5 μg, 7 μg, 7.5 μg, 8 μg, 8.5 μg, 9 μg, 9.5 μg, 10 μg, 10.5 μg, 11 g, 11.5 μg, 12 μg, 12.5 μg, 13 μg, 13.5 μg, 14 μg, 14.5 μg, 15 μg, 15.5 μg, 16 μg, 16.5 μg, 17 μg, 17.5 μg, 18 μg, 18.5 μg, 19 μg, 19.5 μg, 20 μg, 21 μg, 22 μg, 23 μg, 24 μg, 25 g, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 47.5 μg, 50 μg, 52.5 μg, 55 μg, 57.5 μg, 60 μg, 62.5 μg, 65 μg, 67.5 μg, 70 μg, 72.5 μg, 75 μg, 77.5 μg, 80 μg, 82.5 μg, 85 μg, 87.5 μg, 90 μg, 92.5 μg, 95 μg, 97.5 μg, 100 μg, 105 μg, 107.5 μg, 110 μg, 112.5 μg, 115 μg, 117.5 μg, 120 μg, 122.5 μg, 125 μg, 127.5 μg, 130 μg, 135 μg, 140 μg, 150 μg, 155 μg, 160 μg, 165 μg, 170 μg, 175 μg, 180 μg, 185 μg, 190 μg, 195 μg, or 200 μg of the inventive vaccine or vaccine composition, wherein the amount of the inventive vaccine or vaccine composition administered e.g. refers to the amount of the inventive soluble protein complex in the inventive vaccine or vaccine composition, or e.g. to the total amount of the inventive vaccine or vaccine composition administered, e.g. the inventive soluble protein complex, one or more adjuvants and/or one or more pharmaceutically active components as disclosed above.

More specifically, the inventive method of vaccination may comprise administering the inventive vaccine composition or vaccine to a human at least once, twice or three times. Accordingly, the inventive vaccine composition or vaccine as disclosed above, may be administered in any amount as disclosed above, following e.g. any vaccination schedule for a 2 or 3 or more dose vaccination, for example a 0, 1 month schedule, a 0, 2 month schedule, a 0, 3 month schedule, a 0, 4 month schedule, a 0, 5 month schedule or a 0, 6 month schedule for a 2 dose vaccine; a 0, 1 6 month schedule, a 0, 2, 6 month schedule, a 0, 3, 6 month schedule, a 0.4, 6 schedule for a 3 dose vaccination. Thus the second dose may e.g. be administered one month, or e.g. two months, or e.g. three months, or e.g. four months, or e.g. five months, or e.g. six months, or e.g. of about 8 months to about 24 months after the first dose, e.g. 8, 9, 10, 11, 12, 13, 14, 15, 16, 17, 18, 19, 20, 21, 22, 23 or 24 months. Similarly, a third dose may e.g. be administered one month, or e.g. two months, or e.g. three months, or e.g. four months, or e.g. five months, or e.g. six months, or e.g. up to twelve months, or e.g. up to twenty-four months after the second dose.

According to one embodiment, the inventive vaccine composition as disclosed above may be e.g. administered subcutaneously, e.g. in any amount as disclosed above and according to any vaccination schedule, e.g. according to a vaccination schedule as disclosed above. The term “subcutaneous” or “subcutaneously” as used with the inventive method refers to an injection, or delivery of the inventive vaccine or vaccine composition to the layer of skin directly below the dermis and epidermis, which is also collectively referred to as cutis. The subcutaneous administration may be done by any appropriate means, such as e.g. a needle, or e.g. single use injection devices, or e.g. needle-free injection devices such as e.g. Bioject™, Zetajet™ injection devices.

According to a preferred embodiment, the inventive vaccine composition as disclosed above is administered intra-muscularly (i.m.), e.g. in any amount as disclosed above and according to any vaccination schedule, e.g. according to a vaccination schedule as disclosed above. The term “intra-muscular” or “intra-muscularly” as used with the inventive method refers to an injection, or delivery of the inventive vaccine or vaccine composition refers to the injection of a substance directly into a muscle, e.g. preferably to an injection of the inventive vaccine or vaccine composition into a muscle of e.g. the upper thigh, or e.g. vastus lateralis, vastus medialis, or e.g. vastus intermedius muscle, or e.g. deltoid muscle of the arm, or e.g. gluteal muscles. The intra muscular administration may be done by any appropriate means, such as e.g. an injection device, such as e.g. a syringe, or e.g. single use injection devices, e.g. single-use injection syringes. For example, the single-use injection syringes, or single-use injection devices may comprise, pre-filled, a single dose of the inventive vaccine or vaccine composition as disclosed above, in an amount as disclosed above, e.g. of about 2 μg to about 200 μg of the inventive vaccine or vaccine composition, preferably about 20 μg to about 50 μg, or 50 μg to about 200 μg of the inventive vaccine or vaccine composition in a total volume of e.g. about 100μl to about 1000μl, or of about 200 μl, 300 μl, 400 μl, 500μl, 600 μl to about 700 μl, 750 μl, 800 μl, 850 μl, 900 μl, or e.g. of about 300 μl to about 500 μl, or e.g. of about 400 μl to about 650 μl, or e.g. of about 500 μl to about 750 μl. The single-use injection device for i.m. injection of the inventive vaccine or vaccine composition may e.g. be provided in different doses as may be required for the vaccination of newborns, infants or adults, e.g. in lower or larger amounts.

The inventive vaccine or inventive vaccine composition as disclosed above may also be administered in combination with one or more HCMV vaccines, e.g. the inventive vaccine or vaccine composition may be administered in combination with e.g. one or more vaccines selected from the group comprising e.g. gB, or e.g. gB-based vaccines, or HCMV vaccines comprising the AD169 HCMV strain (cf. e.g. Neff et al. (1979) Proc Soc Exp Biol Med, 160:32-7), or e.g. Towne vaccine (cf. e.g. Plotkin et al. (1976) J Infect Dis 134:470-5), or e.g. UL130, UL131 peptide conjugate. vaccines (cf. e.g. Saccoccio et al. (2011) Vaccine 29:2705-11), or e.g. pp65 vaccine (cf. e.g. Berencsi et al., (2001) J Infect Dis 2001; 183:1171-9). The inventive vaccine or vaccine composition may thus be administered as e.g. an admixture of the inventive vaccine or vaccine composition with one or more of the above HCMV vaccines, e.g. as an admixture of the inventive vaccine or inventive vaccine composition with e.g. gB, or with e.g. AD169 HCMV strain vaccine, or with e.g. Towne vaccine, or e.g. with UL130, UL131 peptide conjugate vaccines, or e.g. the inventive vaccine or inventive vaccine composition may e.g. be administered e.g. prior to, or e.g. concurrent with, or e.g. subsequent, with one of the HCMV vaccines as disclosed above, for example, the inventive vaccine or vaccine composition may be e.g. administered 6 months, or e.g. 3 months, or e.g. 1 month, or e.g. 14 days or e.g. 7 days prior to the administration of any of the above HCMV vaccines, or e.g. 1 month, or e.g. 14 days or e.g. 7 days subsequent to the administration of one or more of the above HCMV vaccines, following the vaccination schedule of the respective HCMV vaccine. The term “in combination” as used in the present invention for the administration of the inventive vaccine may e.g. also refer to the separate administration of one of the vaccines as disclosed above with regard to the inventive vaccine, e.g. the term administered in combination may comprise a first administration of the inventive vaccine and a separate, e.g. later administration of one or more vaccines as disclosed above, or the term may also refer to a first administration of a HCMV vaccine as disclosed above, followed by an administration of the inventive vaccine, according to any vaccination schedule as disclosed above.

It is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

### EXAMPLES

### Example 1

### Generation of a DNA Construct Encoding the HCMV Pentameric Protein Complex

In order to obtain the HCMV pentameric protein complex expressed by mammalian cells, the expression system was based on the LONZA GS Gene Expression System™ using the pEE12.4 and pEE6.4 expression vectors as provided by LONZA Biologics. The genes encoding the five subunits of the HCMV pentameric complex (gH, gL, pUL128, pUL130 and pUL131) were engineered and cloned into these vectors and a double gene vector was obtained according to the LONZA GS Gene Expression System™ Manual. The principle thereof is described for example in WO 2008/148519 A2.

Expression of the genes encoding gH and gL was driven by a first human CMV promoter. The genes encoding gH and gL were separated by a sequence encoding the self-processing peptide P2A of the Foot-and-Mouth Disease virus. In order to obtain optimized secretion of the soluble complex, the gH gene was deleted of the transmembrane and cytoplasmic domains. Expression of UL128, UL130 and UL131 was driven by a second human CMV promoter having the same sequence as the first human CMV promoter driving the expression of the genes encoding gH and gL. Genes encoding the self-processing peptide T2A and F2A of the Foot-and-Mouth Disease virus were inserted between UL128 and UL130 and UL130 and UL131, respectively.

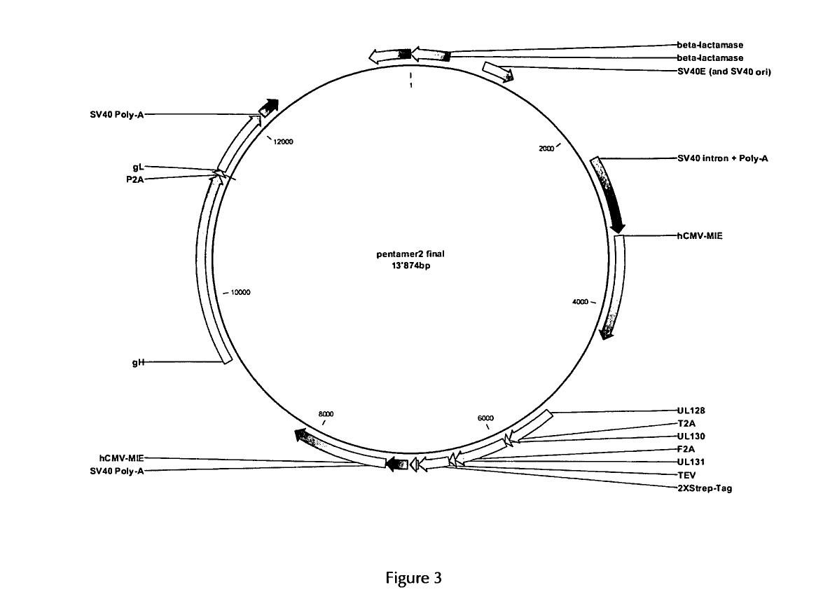

Additional modifications were added to optimize gene transcription, and protein secretion and purification: Firstly, all sequences were codon optimized for expression in mammalian cells. Secondly, the sequence encoding the gH signal peptide was replaced by a sequence encoding the IgG leader sequence MGWSCIILFLVATATGVHS. A sequence encoding a TEV protease cleavage site (ENLYFQG) followed by two Strep-Tags (amino acid sequence WSHPQFEK) was added downstream of UL131. A schematic map of the double gene vector construct is depicted in FIG. 3.

### Example 2

### Generation of a Stable CHO Line Producing the HCMV Pentameric Complex

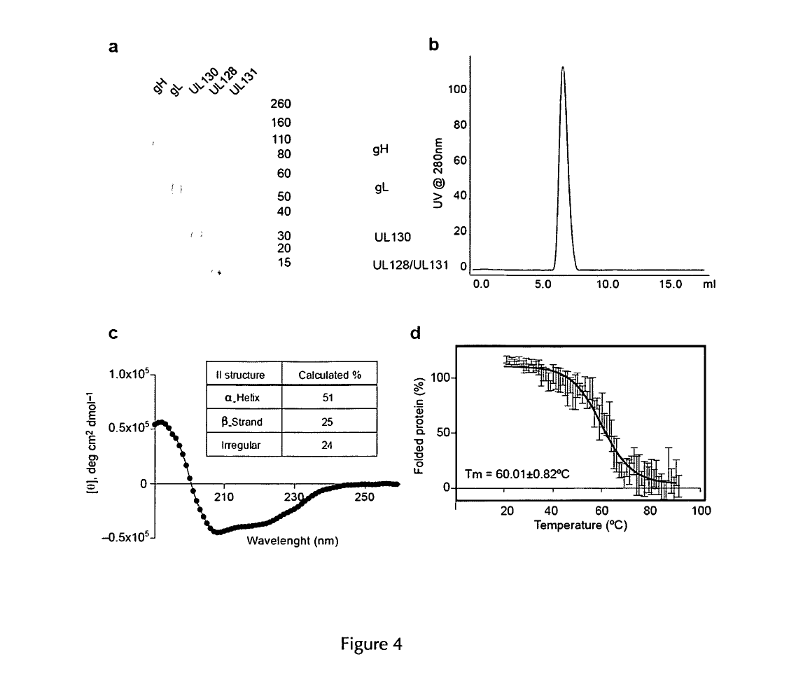

The DNA construct according to Example 1 was used to produce a stable cell line producing a soluble HCMV pentameric complex. CHO-K1SV line (GS-system, licensed by IRB from Lonza) were nucleofected with the prepared vector. Stably transfected CHO clones were obtained. The best clone was further sub-cloned to get a stable cell line with high level production of HCMV pentameric complex. The product of these cell line was characterized (FIG. 4). The preparation of purified, tag-free, HCMV pentameric complex was monodisperse with no signs of aggregation (panel a, b). Secondary structure analysis by circular dichroism revealed that the complex was mainly α-helical and possessed a high stability (Tm˜60° C.), as measured by thermal denaturation analysis (panel c, d).

### Example 3

### Quality Assessment of the Soluble HCMV Pentameric Complex

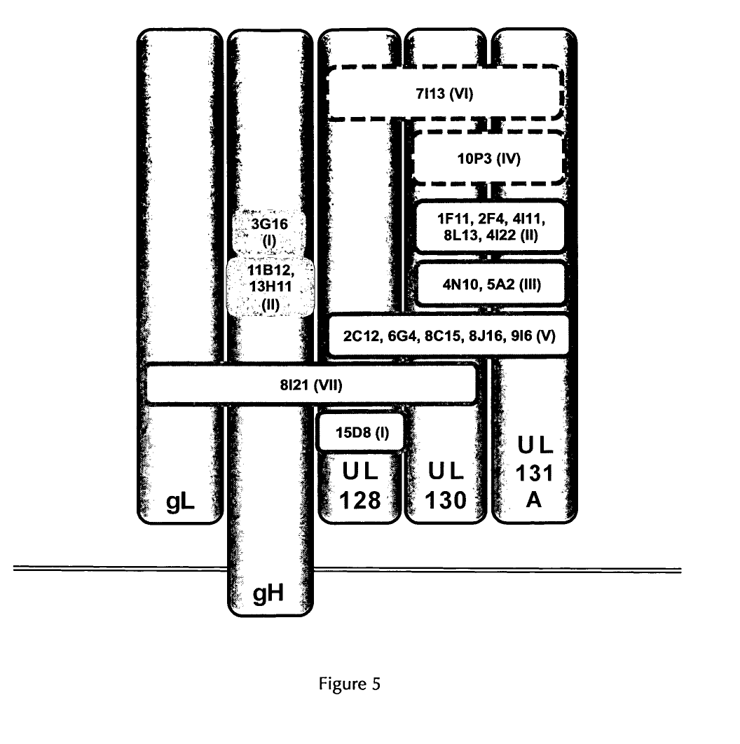

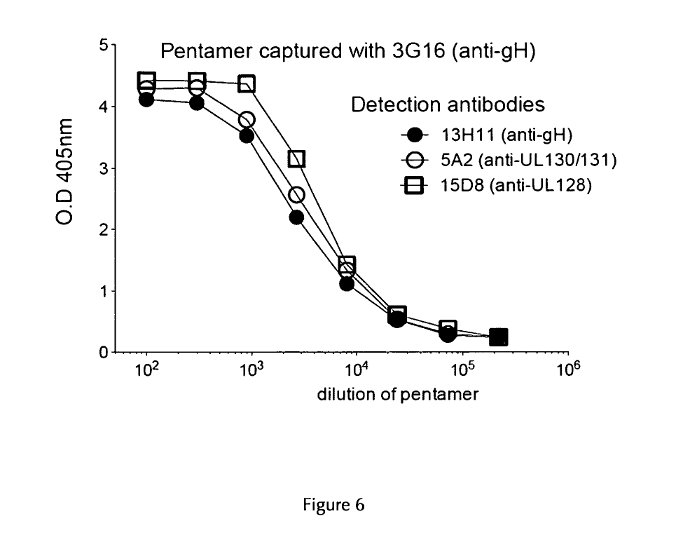

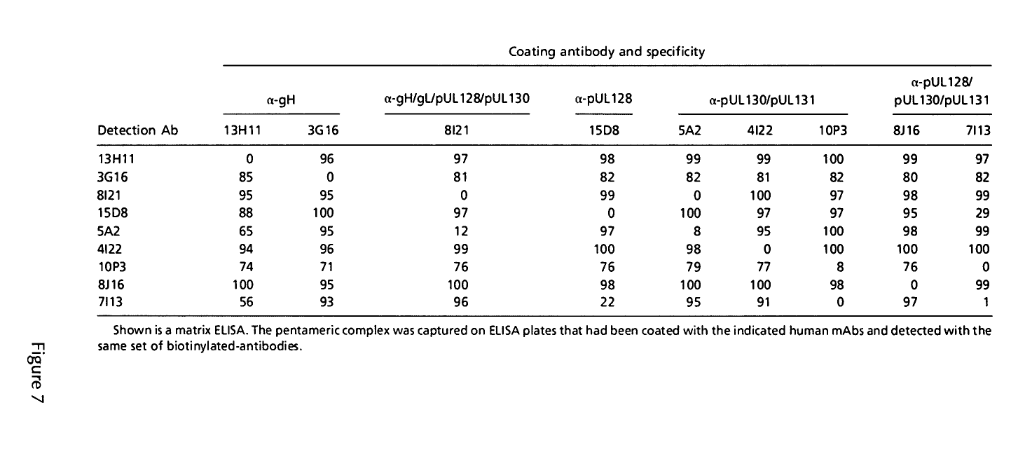

The correct folding of the soluble HCMV pentameric complex was assessed by ELISA using a large panel of human monoclonal antibodies directed against different epitopes displayed on the complex. An overview over the multiple antigenic sites present in the HCMV pentameric complex along with the human neutralizing antibodies specifically binding to these antigenic sites is shown in FIG. 5. A sensitive sandwich ELISA was set up using specific antibodies, namely antibodies 5A2 (anti-pUL130-131), 10P3 (anti-pUL130-131), 8121 (anti-gH/gL/pUL128-130), 13H11 (anti-gH), 3G16 (anti-gH), 15D8 (anti-pUL128), 4122 (anti-pUL130-131), 8J16 (anti-pUL128-130-131), and 7113 (anti-pUL128-130-131), for capture of soluble gHgLpUL128L pentamer to the plastic. Half area 96-well polystyrene plates (high binding, Corning) were coated o.n. at +4° C. with the same set of human antibodies (2 μg/ml) anti-gH, anti-gHgLpUL128pUL130, anti-pUL128, anti-pUL130pUL131 or anti-pUL128pUL130pUL131 mAbs as described above. Plates were blocked with 1% BSA in PBS for 1 h at room temperature. After two washes with PBS-0.05% Tween 20, plates were incubated for 90 min at room temperature with the pentamer in 1% BSA in PBS. Following four washings, primary murinized or biotin-labelled antibodies at (2 microgrammes/ml) diluted in 1% BSA/PBS were added and incubated for 90 min at room temperature. After four washings, alkaline phosphatase-labelled secondary antibody or alkaline phosphatase-labelled streptavidin was added and incubated for 45 min at room temperature, followed by four washings and addition of p-nitrophenyl phosphate substrate solution (Sigma-Aldrich). Absorbance was read at 405 nm after 1 hour and the signal was subtracted from blank (additional plate with the same procedure, but without addition of pentamer). The results are shown in FIG. 6 and FIG. 7. FIG. 6 shows that the soluble purified HCMV pentameric complex obtained according to the present invention is composed by a balanced stoichiometry of the gH and UL128/UL130/UL131 subunits. The results shown in FIG. 7 indicate that (i) all antigenic sites are present in the soluble purified HCMV pentameric complex obtained according to the present invention and (ii) all antigenic sites are present only once in the soluble HCMV pentameric complex obtained according to the present invention, i.e. no multimers occur, since no signal is detected when capture and detection antibodies coincide.

Antibodies specific for epitopes requiring a combination pUL130 and pUL131 or all 5 proteins present in the HCMV pentameric complex (i.e. gH, gL, pUL128, pUL130 and pUL131) reacted with the soluble HCMV pentameric complex produced by the selected CHO cell clone. The antibodies 8L13 (anti-pUL130-131), 5A2 (anti-pUL130-131), 10P3 (anti-pUL130-131), 8121 (anti-gH/gL/pUL128-130), 13H11 (anti-gH), and H1P73 (anti-gH) all bound in ELISA the soluble HCMV pentameric complex present in the CHO supernatant but failed to detect any proteins after the supernatant was immunoprecipitated using an anti-gH (13H11) antibody, indicating that most of the proteins in the supernatants are assembled in the pentameric complex.

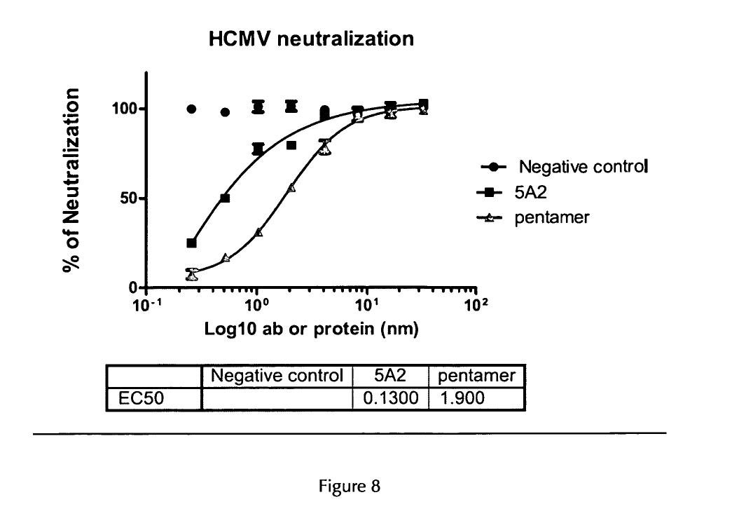

A neutralization assay of HCMV was performed using the epithelial cell lines ARPE 19 as target and either a monoclonal human anti-HCMV antibody (5A2) as control or the soluble HCMV pentameric complex (FIG. 8). The antibody was pre-incubated with the virus for 1 h at 37° C. before addition to the target cells while the complex was pre-incubated with the target cells for 1 h at 37° C. before addition of the virus. Both the antibody and the soluble pentameric complex interfere with virus entry, with IC50 of 0.13 nM or 1.9 nM, respectively. This data further supports the concept that the soluble HCMV pentameric complex has the correct folding capable of binding to the cellular receptor used by the virus to infect target cells.

### Example 4

High Neutralizing Antibody Titers Elicited In Vivo by a Soluble gHgLpUL128L Pentameric Complex Vaccine

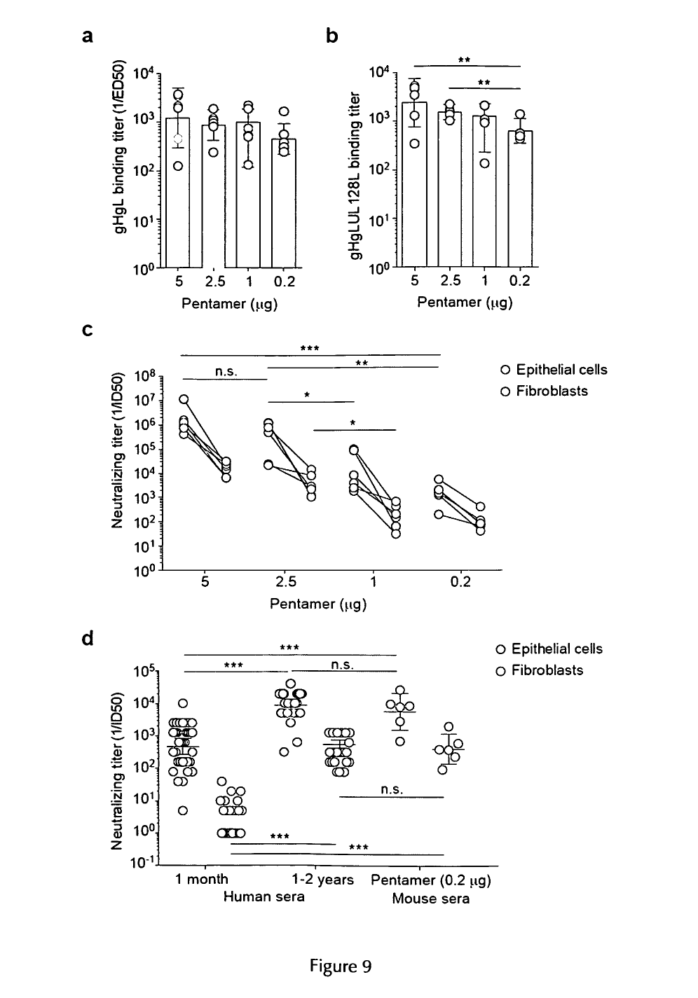

The ability of the HCMV pentameric complex produced as in Example 2 to induce an immune response in vivo was assessed by immunizing Balb/c mice subcutaneously into flank on day 0. Two booster immunization were given on day +14 and day +28. Sera were analyzed on day +40. Dose-finding experiments showed that high serum binding titers to gHgL or gHgLpUL128L were induced at doses as low as 1 μg/mouse (FIG. 9a, b). Extraordinarily high serum neutralizing titers of HCMV infection of epithelial cells were induced at a dose of 5 μg/mouse and 2.5 μg/mouse. These titers were significantly higher to that induced by a dose of 0.2 μg/mouse (FIG. 9c). Sera of mice immunized 40 days before with 0.2 μg pentamer had neutralizing titers that inhibited infection of both epithelial cells or fibroblasts significantly higher to those found in the sera of patients 1 months after HCMV infection (FIG. 9d).

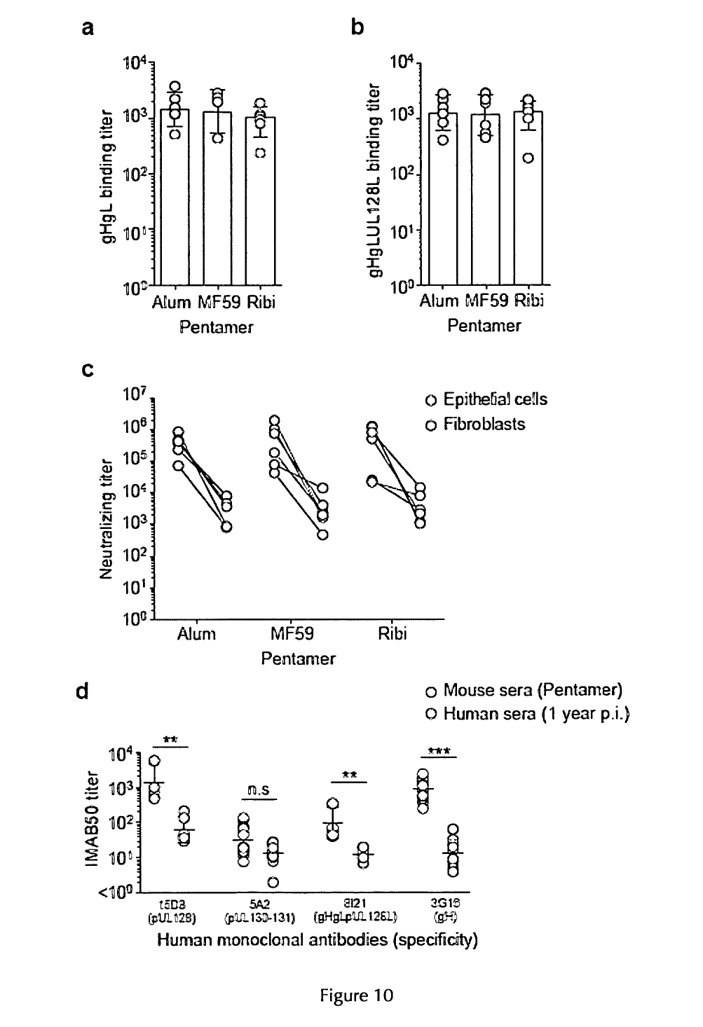

To evaluate different adjuvants, mice were immunized with the HCMV pentameric complex (2.5 μg/mouse) formulated in three different clinically used adjuvants: Alum, MF59, and Ribi. When normalized on total IgG serum content, the three preparations were equally effective in inducing high serum binding and neutralizing titers (FIG. 10).

### Example 5

### The HCMV Pentameric Complex Vaccine Elicits an Antibody Response of High Specific Activity

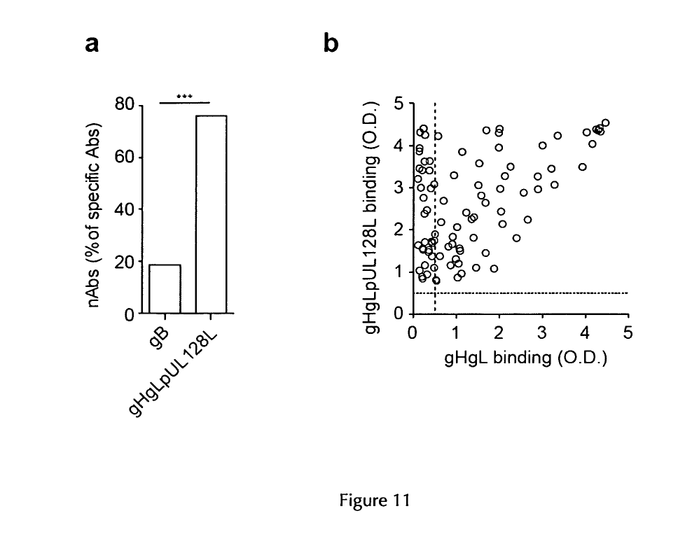

To precisely define the specific activity of the antibody response induced by the soluble gB and the soluble HCMV pentameric complex vaccines, memory B cells from immunized mice were fused with myeloma cells and monoclonal antibodies were isolated from hybridomas. Three hundred forty two (342) monoclonal antibodies that bound to soluble gB were obtained from 4 gB-vaccinated mice, while 247 monoclonal antibodies that bound to the soluble HCMV pentameric complex were obtained from 4 complex-vaccinated mice (FIG. 11a).). Importantly, however, while only a minor fraction of antibodies elicited by the gB vaccine was capable of neutralizing HCMV infection (19.9±4.2%, range 15%-20%), the large majority of antibodies elicited by the HCMV pentameric complex vaccine was neutralizing (75.7±11.5%, range 63%-91%) (FIG. 11b). Thus, the HCMV pentameric complex vaccine preferentially elicit neutralizing antibodies and has therefore a higher specific activity than the gB vaccine.

### Example 6

### The HCMV Pentameric Complex Vaccine Elicits a Broad Repertoire of Antibodies Neutralizing Infection of Both Fibroblasts as Well as Epithelial, Endothelial, and Myeloid Cells

The fine specificity and functional properties of the monoclonal antibodies isolated from mice immunized with the HCMV pentameric vaccine was studied using binding and neutralization assays. A large fraction of the antibodies (67%) was specific for gHgL, since they bound to both gHgL dimer and gHgLpUL128L pentameric complexes and neutralized infection of both fibroblasts and epithelial cells, with IC80 values in the nanomolar range (IC80 0.5-10 nM). The remaining 33% of the antibodies bound to the gHgLpUL128L pentameric complexe and selectively neutralized infection of epithelial cells in the picomolar range (IC80 0.8-500 μM). A side-to-side comparison showed that mouse antibodies elicited by the HCMV pentameric complex vaccine and human antibodies induced by natural HCMV infection had comparable potencies and fine specificities, as determined by their capacity to bind to cells transfected with gH, gL UL128, UL130, and UL131 genes in different combinations. In addition, cross-competition experiments showed that some of the most potent neutralizing antibodies produced by vaccinated mice targeted novel antigenic sites on the pentamer that were not identified using the large panel of human monoclonal antibodies previously isolated (Table 1/FIG. 9). The above findings demonstrate that the gHgLpUL128L pentameric vaccine can elicit a strong antibody response that is largely composed of potent neutralizing antibodies that inhibit HCMV infection of fibroblasts, epithelial, endothelial, and myeloid I cells similar to those produced in humans in HCMV infection.

TABLE 1
Characterization of monoclonal antibodies (mAbs) from mice immunized with soluble
HCMV pentameric complex

| | Target | Log | | Cross- |
|---|---|---|---|---|
| mAb | cells | IC₈₀ | Target antigen | competing |
| m-Ab P25 | Epithelial | −12.1 | pUL128pUL130pUL131 | — |
| m-Ab P40 | Epithelial | −11.4 | pUL128pUL130pUL131 | — |
| m-Ab P38 | Epithelial | −11.3 | pUL128pUL130pUL131 | — |
| m-Ab P39 | Epithelial | −11.2 | pUL128pUL130pUL131 | — |
| m-Ab P53 | Epithelial | −10.9 | pUL128pUL130pUL131 | — |
| m-Ab P31 | Epithelial | −10.9 | pUL128pUL130pUL131 | — |
| m-Ab P42 | Epithelial | −10.6 | pUL128pUL130pUL131 | h-mAb 8J16 |
| m-Ab P2 | Epithelial | −10.8 | gHgLpUL128 | h-mAb 15D8 |
| m-Ab P30 | Epithelial | −10.4 | pUL130pUL131 | h-mAb 4I22 |
| m-Ab P37 | Epithelial | −10.4 | gHgLpUL128 | h-mAb 15D8 |
| m-Ab P46 | Epithelial | −9.5 | pUL128pUL130pUL131 | h-mAb 4I22 |
| m-Ab P7 | Epithelial | −9.5 | pUL128pUL130pUL131 | — |
| m-Ab P16 | Epithelial | −9.3 | UL128 | h-mAb 5A2 |
| | | | | h-mAb 8I21 |
| m-Ab D1 | Epithelial/Fibroblasts | −9.3 | gH | h-mAb 13H11 |
| m-Ab D7 | Epithelial/Fibroblasts | −8.9 | gH | — |
| m-Ab D12 | Epithelial/Fibroblasts | −8.9 | gH | — |
| m-Ab D13 | Epithelial/Fibroblasts | −8.4 | gH | — |
| h-Ab 8J16 | Epithelial | −12.3 | pUL128pUL130pUL131 | — |
| h-Ab 8L13 | Epithelial | −11.6 | pUL130pUL131 | — |
| h-Ab 7I13 | Epithelial | −11.0 | pUL128pUL130pUL131 | h-mAb 10P3 |
| | | | | h-mAb 15D8 |
| h-Ab 15D8 | Epithelial | −11.0 | pUL128 | h-mAb 7I13 |
| h-Ab 10P3 | Epithelial | −10.5 | pUL130pUL131 | h-mAb 7I13 |
| h-Ab 5A2 | Epithelial | −10.0 | pUL130pUL131 | h-mAb 8I21 |
| h-Ab 8I21 | Epithelial | −9.5 | gHgLpUL128pUL130 | h-mAb 5A2 |
| h-Ab 13H11 | Epithelial/Fibroblasts | −8.6 | gH | — |

Mouse monoclonal antibodies (m-Abs) and human monoclonal antibodies (h-Abs) are grouped according to their ability to neutralize HCMV infection of epithelial cells only or epithelial cells and fibroblasts. Shown are the log IC80 values, corresponding to the concentration that inhibits 80% infection. Ab target antigen was determined using HEK293T cells transfected with different combination of HCMV genes. Cross-competition ELISA assays were performed to identify the m-Abs binding to overlapping sites bound by a panel of human monoclonal antibodies previously isolated (Macagno et al, J Virol. 2010 January; 84(2):1005-13. doi: 10.1128/JVI.01809-09).

1. A vector for expressing HCMV glycoproteins in a mammalian cell, wherein the vector comprises a transcription system comprising
(i) at least one promoter operable in a mammalian cell and operably linked to
(ii) at least one open reading frame comprising at least one nucleotide sequence selected from the group consisting of nucleotide sequences encoding gH, gL, UL128, UL130 and UL131 or sequence variants thereof,
whereby the vector comprises a nucleotide sequence encoding gH, a nucleotide sequence encoding gL, a nucleotide sequence encoding UL128, a nucleotide sequence encoding UL130 and a nucleotide sequence encoding UL131 or the sequence variants thereof.

2. The vector according to claim 1, wherein:
(a) said vector is not a self-replicating RNA molecule nor does it comprise a self-replicating RNA molecule;
(b) said vector is not an alphavirus replicon nor does it comprise an alphavirus replicon;
(c) said vector does not comprise any sequence encoding an alphavirus non-structural protein such as NSP1, NSP2, NSP3 and NSP4;
(d) said vector is not packaged into viral replicon particles, is not encapsulated in lipid nanoparticles, and is not formulated with CMF34;
(e) said vector is not derived from and not comprised by a bacterial artificial chromosome (BAC) construct;
(f) said vector is not an MVA-derived vector;
(g) said vector does not comprise a sequence encoding a viral capsid or capsid precursor protein;
(h) the backbone of said vector is neither pRBT136 nor pRBT393; and/or
(i) said vector is not derived from or is not a retroviral vector, a lentiviral vector, an adenoviral vector, or an adeno-associated viral vector.

3.-6. (canceled)

7. The vector according to claim 1, wherein said vector is a DNA construct.

8. The vector according to claim 1, wherein the nucleotide sequences encoding gH, gL, UL128, UL130 and UL131 are nucleotide sequences encoding the amino acid sequences according to SEQ ID NO:21, SEQ ID NO:25, SEQ ID NO:3, SEQ ID NO:7 and SEQ ID NO: 11 or sequence variants thereof.

9. The vector according to claim 8, wherein the nucleotide sequences encoding the amino acid sequences according to SEQ ID NO:21, SEQ ID NO:25, SEQ ID NO:3, SEQ ID NO:7 and SEQ ID NO: 11 are nucleotide sequences according to SEQ ID NO:22, SEQ ID NO:26, SEQ ID NO:4, SEQ ID NO:8 and SEQ ID NO:12 or sequence variants thereof.

10. (canceled)

11. The vector according to claim 1, wherein the vector comprises:
(a) one promoter operably linked to an open reading frame, which comprises a nucleotide sequence encoding gH, a nucleotide sequence encoding gL, a nucleotide sequence encoding UL128, a nucleotide sequence encoding UL130 and a nucleotide sequence encoding UL131 or sequence variants thereof; or
(b) two promoters, each of which is operably linked to a nucleotide sequence comprising a first open reading frame and a second open reading frame, wherein the first open reading frame comprises 1 to 4 nucleotide sequences encoding a gH, gL, UL128, UL130 and UL131 or sequence variants thereof, and the second open reading frame comprises nucleotide sequences encoding those of gH, gL, UL128, UL130 and UL131 or sequence variants thereof, that are not contained in the first open reading frame; or
(c) no more than two promoters operably linked to at least one open reading frame comprising at least one nucleotide sequence selected from the group consisting of a nucleotide sequence encoding gH, a nucleotide sequence encoding gL, a nucleotide sequence encoding UL128, a nucleotide sequence encoding UL130, a nucleotide sequence encoding UL131 or sequence variants thereof.

12. (canceled)

13. The vector according to claim 10, wherein the vector comprises a transcription system comprising:
(i) a first promoter operable in a mammalian cell and operably linked to
(ii) a first open reading frame comprising a nucleotide sequence encoding gH or sequence variants thereof and a nucleotide sequence encoding gL or sequence variants thereof,
(iii) a second promoter operable in a mammalian cell and operably linked to
(iv) a second open reading frame comprising a nucleotide sequence encoding UL128 or sequence variants thereof, a nucleotide sequence encoding UL130 or sequence variants thereof and a nucleotide sequence encoding UL131 or sequence variants thereof.

14. The vector according to claim 13, wherein the first and/or the second open reading frame comprise(s):
(a) a nucleotide sequence further encoding at least one of a linker sequence, a tag sequence, a peptide cleavage site, a ribosomal skipping site and a signal peptide; and/or
(b) at least one nucleotide sequence encoding an amino acid sequence selected from the group consisting of SEQ ID NO:5, SEQ ID NO:9, and SEQ ID NO:23 or sequence variants thereof, in particular the sequence variants SEQ ID NO:27 or SEQ ID NO:29.

15. The vector according to claim 14, wherein the tag sequence is selected from a His-Tag or a Strep-Tag sequence, the signal peptide sequence is selected from an IgG signal peptide sequence, the cleavage site is selected from a TEV site, the ribosomal skipping site is selected from the sequence motif D-V/I-E-X-N-P-G≠P, and the linker sequence is selected from a GS linker.

16. (canceled)

17. The vector according to claim 1, wherein within each open reading frame the nucleotide sequences selected from the group consisting of a nucleotide sequence encoding gH, a nucleotide sequence encoding gL, a nucleotide sequence encoding UL128, a nucleotide sequence encoding UL130, a nucleotide sequence encoding UL131 and sequence variants thereof are separated from each other by a nucleotide sequence encoding a ribosomal skipping site.

18. The vector according to claim 13, wherein:
(a) the first and the second open reading frames each comprise at least one nucleotide sequence encoding a ribosomal skipping site having an amino acid selected from SEQ ID NO:23, SEQ ID NO:27 and SEQ ID NO:29 or sequence variants thereof;
(b) the first open reading frame comprises a nucleotide sequence encoding a ribosomal skipping site having an amino acid sequence according to SEQ ID NO:23, SEQ ID NO:27, SEQ ID NO:29 or sequence variants thereof; and the second open reading frame comprises at least one nucleotide sequence encoding a ribosomal skipping site having an amino acid sequence according to SEQ ID NO:5, SEQ ID NO:9, SEQ ID NO:23, SEQ ID NO:27, SEQ ID NO:29 or sequence variants thereof; or
(c) the first open reading frame comprises a nucleotide sequence encoding a ribosomal skipping site having an amino acid sequence according to SEQ ID NO:23, SEQ ID NO:27, SEQ ID NO:29 or sequence variants thereof; and the second open reading frame comprises at least one nucleotide sequence encoding a ribosomal skipping site having an amino acid sequence according to SEQ ID NO:5, SEQ ID NO:9 or sequence variants thereof.

19. (canceled)

20. The vector according to claim 18, wherein the ribosomal skipping site having an amino acid sequence according to SEQ ID NO:23, SEQ ID NO:27, SEQ ID NO:29, or sequence variants thereof is located in the first open reading frame between the nucleotide sequence encoding gH or sequence variants thereof and the nucleotide sequence encoding gL or sequence variants thereof; and wherein the second open reading frame comprises a nucleotide sequence encoding a first ribosomal skipping site and a second ribosomal skipping site, wherein the first ribosomal skipping site has an amino acid sequence according to SEQ ID NO:5, SEQ ID NO:9, SEQ ID NO:23, SEQ ID NO:27, SEQ ID NO:29 or sequence variants thereof and is located in the second open reading frame between the nucleotide sequence encoding UL128 or sequence variants thereof and the nucleotide sequence encoding UL130 or sequence variants thereof, and the second ribosomal skipping site has an amino acid sequence according to SEQ ID NO:5, SEQ ID NO:9, SEQ ID NO:23, SEQ ID NO:27, SEQ ID NO:29 or sequence variants thereof and is located in the second open reading frame between the nucleotide sequence encoding UL130 or sequence variants thereof and the nucleotide sequence encoding UL131 or sequence variants thereof.

21. The vector according to claim 13, wherein the second open reading frame comprises the nucleotide sequences encoding UL128, UL130 and UL131, or sequence variants thereof, and at least one nucleotide sequence encoding an amino acid sequence selected from the group consisting of SEQ ID NO: 13, SEQ ID NO:15, SEQ ID NO:17 and SEQ ID NO:41 or sequence variants thereof.

22. The vector according to claim 1, wherein the vector comprises a nucleotide sequence encoding a tag sequence that is located no more than 100 nucleotides downstream of the 3′-end of a nucleotide sequence encoding UL131, whereby the nucleotide sequence encoding the tag sequence is optionally separated from nucleotide sequence encoding UL131 by a nucleotide sequence encoding a linker and/or a nucleotide sequence encoding a peptide cleavage site.

23. The vector according to claim 22, wherein the vector does not comprise a nucleotide sequence encoding the tag sequence that is located adjacently to the 3′-end of a nucleotide sequence encoding gH, gL, UL128 or UL130.

24. The vector according to claim 22, wherein the vector comprises a nucleotide sequence encoding the tag sequence, the peptide cleavage site and the linker sequence, wherein the tag sequence comprises or consists of an amino acid sequence according to SEQ ID NOs: 17, 39, 41 or sequence variants thereof, the peptide cleavage site comprises or consists of an amino acid sequence according to SEQ ID NO: 13 or sequence variants thereof, and the linker sequence comprises or consists of an amino acid sequence according to SEQ ID NO: 15 or sequence variants thereof.

25. The vector according to claim 24, wherein the vector comprises a nucleotide sequence encoding the tag sequence, which comprises or consists of an nucleotide sequence according to SEQ ID NOs: 18, 40, and 42, or sequence variants thereof, a nucleotide sequence encoding the peptide cleavage site, which comprises or consists of a nucleotide sequence according to SEQ ID NO: 14 or sequence variants thereof, and a nucleotide sequence encoding the linker sequence, which comprises or consists of a nucleotide sequence according to SEQ ID NO: 16 or sequence variants thereof.

26. The vector according to claim 13, wherein
(a) the first open reading frame comprises a nucleic acid sequence, operably linked in 5′ to 3′ direction, containing SEQ ID NO:20, SEQ ID NO:22, SEQ ID NO:24 and SEQ ID NO:26, or sequence variants thereof; or a nucleic acid sequence, operably linked in 5′ to 3′ direction, containing SEQ ID NO:20, SEQ ID NO:36, SEQ ID NO:28 and SEQ ID NO:38, or sequence variants thereof; or a nucleic acid sequence containing SEQ ID NO:20, SEQ ID NO:36, SEQ ID NO:30 and SEQ ID NO:38, or sequence variants thereof, and/or
(b) a second open reading frame comprises a nucleic acid sequence, operably linked in 5′ to 3′ direction, containing SEQ ID NO:2, SEQ ID NO:4, SEQ ID NO:24, SEQ ID NO:8, SEQ ID NO:24 and SEQ ID NO: 12, or sequence variants thereof, or a nucleic acid sequence, operably linked in 5′ to 3′ direction, containing SEQ ID NO:20, SEQ ID NO:4, SEQ ID NO:24, SEQ ID NO:8, SEQ ID NO:24, SEQ ID NO:12, SEQ ID NO:14, SEQ ID NO:16, SEQ ID NO:18 and SEQ ID NO:42, or sequence variants thereof, or a nucleic acid sequence, operably linked in 5′ to 3′ direction, containing SEQ ID NO:2, SEQ ID NO:4, SEQ ID NO:24, SEQ ID NO:8, SEQ ID NO:10, SEQ ID NO:12, SEQ ID NO: 14, SEQ ID NO:16 and SEQ ID NO: 18, or sequence variants thereof, or a nucleic acid sequence, operably linked in 5′ to 3′ direction, containing SEQ ID NO:20, SEQ ID NO:4, SEQ ID NO:28, SEQ ID NO:32, SEQ ID NO:28 and SEQ ID NO:34, or sequence variants thereof, or a nucleic acid sequence, operably linked in 5′ to 3′ direction, containing SEQ ID NO:20, SEQ ID NO:4, SEQ ID NO:30, SEQ ID NO:32, SEQ ID NO:30 and SEQ ID NO:34, or sequence variants thereof, or a nucleic acid sequence, operably linked in 5′ to 3′ direction, containing SEQ ID NO:20, SEQ ID NO:4, SEQ ID NO:28, SEQ ID NO:32, SEQ ID NO:28, SEQ ID NO:34, SEQ ID NO: 14, SEQ ID NO: 16, SEQ ID NO:40 and SEQ ID NO:42, or sequence variants thereof, or a nucleic acid sequence, operably linked in 5′ to 3′ direction, containing SEQ ID NO:20, SEQ ID NO:4, SEQ ID NO:30, SEQ ID NO:32, SEQ ID NO:30, SEQ ID NO:34, SEQ ID NO:14, SEQ ID NO:16, SEQ ID NO:40 and SEQ ID NO:42, or sequence variants thereof, or a nucleic acid sequence, operably linked in 5′ to 3′ direction, containing SEQ ED NO:20, SEQ ID NO:4, SEQ ID NO:28, SEQ ID NO:32, SEQ ID NO:28, SEQ ID NO:34, SEQ ID NO: 14, SEQ ID NO: 16 and SEQ ID NO:40, or sequence variants thereof, or a nucleic acid sequence, operably linked in 5′ to 3′ direction, containing SEQ ID NO:20, SEQ ID NO:4, SEQ ID NO:30, SEQ ID NO:32, SEQ ID NO:30, SEQ ID NO:34, SEQ ID NO: 14, SEQ ID NO: 16 and SEQ ID NO:40, or sequence variants thereof.

27. (canceled)

28. The vector according to claim 1, wherein the vector comprises the nucleotide sequence, operably linked in 5′ to 3′ direction, containing SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 and SEQ ID NO:26, or sequence variants thereof.

29. The vector according to claim 1, wherein a first open reading frame and/or second open reading frame comprise:
(a) a nucleotide sequence, operably linked in 5′ to 3′ direction, containing SEQ ID NO:20, SEQ ID NO:4, SEQ ID NO:28, SEQ ID NO:32, SEQ ID NO:28, SEQ ID NO:34, SEQ ID NO:20, SEQ ID NO:36, SEQ ID NO:28 and SEQ ID NO:38, or sequence variants thereof; or
(b) a nucleotide sequence, operably linked in 5′ to 3′ direction, containing SEQ ID NO:20, SEQ ID NO:4, SEQ ID NO:30, SEQ ID NO:32, SEQ ID NO:30, SEQ ID NO:34, SEQ ID NO:20, SEQ ID NO:36, SEQ ID NO:30 and SEQ ID NO:38, or sequence variants thereof.

30. (canceled)

31. The vector according to claim 1, wherein the vector comprises, operably linked in 5′ to 3′ direction:
(a) a nucleotide sequence containing SEQ ID NO:20, SEQ ID NO:4, SEQ ID NO:28, SEQ ID NO:32, SEQ ID NO:28, SEQ ID NO:34, SEQ ID NO: 14, SEQ ID NO: 16, SEQ ID NO:40 and SEQ ID NO:42, or sequence variants thereof, and a nucleotide sequence containing SEQ ID NO:20, SEQ ID NO:36, SEQ ID NO:28 and SEQ ID NO:38, or sequence variants thereof;
(b) a nucleotide sequence containing SEQ ID NO:20, SEQ ID NO:4, SEQ ID NO:30, SEQ ID NO:32, SEQ ID NO:30, SEQ ID NO:34, SEQ ID NO: 14, SEQ ID NO: 16, SEQ ID NO:40 and SEQ ID NO:42, or sequence variants thereof, and a nucleotide sequence containing SEQ ID NO:20, SEQ ID NO:36, SEQ ID NO:30 and SEQ ID NO:38, or sequence variants thereof;
(c) a nucleotide sequence containing SEQ ID NO:20, SEQ ID NO:4, SEQ ID NO:28, SEQ ID NO:32, SEQ ID NO:28, SEQ ID NO:34, SEQ ID NO: 14, SEQ ID NO: 16, SEQ ID NO:40, SEQ ID NO:20, SEQ ID NO:36, SEQ ID NO:28 and SEQ ID NO:38, or sequence variants thereof; or
(d) a nucleotide sequence containing SEQ ID NO:20, SEQ ID NO:4, SEQ ID NO:30, SEQ ID NO:32, SEQ ID NO:30, SEQ ID NO:34, SEQ ID NO:14, SEQ ID NO: 16, SEQ ID NO:40, SEQ ID NO:20, SEQ ID NO:36, SEQ ID NO:30 and SEQ ID NO:38, or sequence variants thereof.

32.-34. (canceled)

35. The vector according to claim 1, wherein the vector comprises a first or a second promoter selected from a viral promoter or a non-viral promoter or both.

36. The vector according to claim 35, wherein a first and/or a second promoter is a MCMV, a HCMV, a SV40, a HSV-TK, an EF1-1α or a PGK promoter, or hCMV-MIE promoter.

37.-38. (canceled)

39. A gene expression system, comprising at least one mammalian cell containing a vector according to claim 1.

40.-42. (canceled)

43. The gene expression system according to claim 39, wherein the at least one mammalian cell is selected from the group comprising BHK, DUXB11, CHO-DG44, CHO-K1, CHO-K1SV, CHO-S, CHO-DXB11, CHO-K1SV GS knock-out (CHO-K1SV KO), CAP, PER.C6, NS0, Sp2/0, HEK293 T, HEK 293-F, HEK 6E, HEK293 EBNA, CAP-T, HELA, CVI, COS, R1610, BALBC/3T3, HAK, BFA-1c1BPT, RAJI, HT-1080, and HKB-11.

44.-46. (canceled)

47. A soluble protein complex encoded by a nucleotide sequence according to claim 1.

48. The soluble protein complex according to claim 47, wherein the nucleotide sequence encodes the protein complex comprising an amino acid sequence containing SEQ ID NO:3, SEQ ID NO:7, SEQ ID NO:11, SEQ ID NO:21 and SEQ ID NO:25, or sequence variants thereof.

49. A composition, comprising the vector according to claim 1 and a pharmaceutically acceptable carrier.

50. The composition to claim 49, further comprising one or more adjuvants.

51. The composition according to claim 49, wherein the adjuvant is selected from the group consisting of Alum, Ribi (Monophosphoryl lipid A, MPL), and MF59.

52.-58. (canceled)

59. A process for preparing a vaccine composition, comprising the following steps:
(a) culturing a mammalian producer cell transfected with the vector according to claim 1;
(b) harvesting a HCMV pentamer comprising an amino acid sequence containing SEQ ID NO:3, SEQ ID NO:7, SEQ ID NO: 11, SEQ ID NO:21 and SEQ ID NO:25, or sequence variants thereof, from the mammalian producer cell, optionally purifying the harvested HCMV pentamer; and
(c) formulating the harvested HCMV pentamer into a liquid or solid formulation.

60.-71. (canceled)

72. A nucleic acid, comprising SEQ ID NO:2, SEQ ID NO:4, SEQ ID NO:8, SEQ ID NO:12, SEQ ID NO:22, and SEQ ID NO:26, or SEQ ID NO:2, SEQ ID NO:4, SEQ ID NO:8, SEQ ID NO:12, SEQ ID NO:22, SEQ ID NO:26, SEQ ID NO:14, SEQ ID NO: 16 and SEQ ID NO:42, or sequence variants thereof.

73. The nucleic acid according to claim 72, further comprising SEQ ID NO:6 and/or SEQ ID NO:10 and/or SEQ ID NO:24, and/or SEQ ID NO:28, and/or SEQ ID NO:30, or sequence variants thereof.

74. The nucleic acid according to claim 72, comprising operably linked in 5′ to 3′ direction:
(a) a nucleic acid sequence containing SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 and SEQ ID NO:26, or sequence variants thereof;
(b) a nucleic acid sequence containing SEQ ID NO:20, SEQ ID NO:4, SEQ ID NO:28, SEQ ID NO:32, SEQ ID NO:28, SEQ ID NO:34, SEQ ID NO:20, SEQ ID NO:36, SEQ ID NO:28 and SEQ ID NO:38 or sequence variants thereof;
(c) a nucleic acid sequence containing SEQ ID NO:20, SEQ ID NO:4, SEQ ID NO:30, SEQ ID NO:32, SEQ ID NO:30, SEQ ID NO:34, SEQ ID NO:20, SEQ ID NO:36, SEQ ID NO:30 and SEQ ID NO:38, or sequence variants thereof;
(d) a nucleic acid sequence containing SEQ ID NO:20, SEQ ID NO:4, SEQ ID NO:28, SEQ ID NO:32, SEQ ID NO:28, SEQ ID NO:34, SEQ ID NO:14, SEQ ID NO:16, SEQ ID NO:40, SEQ ID NO:42, SEQ ID NO:20, SEQ ID NO:36, SEQ ID NO:28 and SEQ ID NO:38, or sequence variants thereof;
(e) a nucleic acid sequence containing SEQ ID NO:20, SEQ ID NO:4, SEQ ID NO:30, SEQ ID NO:32, SEQ ID NO:30, SEQ ID NO:34, SEQ ID NO: 14, SEQ ID NO: 16, SEQ ID NO:40, SEQ ID NO:42, SEQ ID NO:20, SEQ ID NO:36, SEQ ID NO:30 and SEQ ID NO:38, or sequence variants thereof;
(f) a nucleic acid sequence containing SEQ ID NO:20, SEQ ID NO:4, SEQ ID NO:28, SEQ ID NO:32, SEQ ID NO:28, SEQ ID NO:34, SEQ ID NO: 14, SEQ ID NO: 16, SEQ ID NO:40, SEQ ID NO:20, SEQ ID NO:36, SEQ ID NO:28 and SEQ ID NO:38, or sequence variants thereof; or
(g) a nucleic acid sequence containing SEQ ID NO:20, SEQ ID NO:4, SEQ ID NO:30, SEQ ID NO:32, SEQ ID NO:30, SEQ ID NO:34, SEQ ID NO: 14, SEQ ID NO:16, SEQ ID NO:40, SEQ ID NO:20, SEQ ID NO:36, SEQ ID NO:30 and SEQ ID NO:38, or sequence variants thereof.

75.-80. (canceled)

81. The nucleic acid according to claim 72, comprising a nucleotide sequence according to SEQ ID NO:44, or SEQ ID NO:46, or SEQ OD NO:48, or SEQ ID NO:50, or sequence variants thereof.

82.-84. (canceled)

85. A method for vaccination, wherein the method comprises administering to a human a therapeutically effective amount of the composition according to claim 49, optionally further comprising a distinct HCMV vaccination compound/complex.

86.-89. (canceled)

90. A host cell, comprising a mammalian cell containing a stable transcription system comprising a promoter operably linked to a polynucleotide sequence encoding gH, gL, UL128, UL130 and/or UL131, or sequence variants thereof,
whereby the mammalian cell containing the stable transcription system expresses a HCMV pentamer comprising the amino acid sequences according to SEQ ID NO:3, SEQ ID NO:7, SEQ ID NO: 11, SEQ ID NO:21 and SEQ ID NO:25, or sequence variants thereof.


SEQUENCE LISTING (59 sequences)

SEQ ID NO: 1 - protein, 22 aa, Artificial Sequence
  signal peptide
MSPKDLTPFLTALWLLLGHSRV

SEQ ID NO: 2 - DNA, 66 bp, Artificial Sequence
  signal sequence
atgtctcctaaagacctgactccattcctgaccgccctgtggctgctgctggggcattcccgtgtc

SEQ ID NO: 3 - protein, 151 aa, Artificial Sequence
  UL128
PRVRAEECCEFINVNHPPERCYDFKMCNRFTVAYVLRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 4 - DNA, 453 bp, Artificial Sequence
  UL128
cctcgtgtccgtgctgaagagtgctgtgagttcatcaacgtgaatcacccccctgaaaggtgctacgacttcaagatgtgtaataggttcaccgtggcttacgtcctgcgctgcccagatggcgaagtgtgttattcccccgagaagactgcagaaatcaggggcattgtcaccacaatgacacacagtctgactcggcaggtggtccataacaaactgacctcatgcaactacaatcctctgtatctggaagccgacggtcgaatccgttgtggcaaggtcaatgataaagctcagtacctgctgggagccgctggctcagtcccatacagatggattaacctggagtatgacaagatcacccgcattgtgggactggatcagtacctggaaagcgtgaagaaacacaagagactggacgtctgccgcgcaaaaatgggatatatgctgcag

SEQ ID NO: 5 - protein, 18 aa, Artificial Sequence
  T2A
EGRGSLLTCGDVEENPGP

SEQ ID NO: 6 - DNA, 54 bp, Artificial Sequence
  T2A
gaggggagagggtccctgctgacatgtggcgacgtggaggaaaaccccggccct

SEQ ID NO: 7 - protein, 186 aa, Artificial Sequence
  UL130v1
WSTLTANQNPSPPWSKLTYSKPHDAATFYCPFLYPSPPRSPSQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLI

SEQ ID NO: 8 - DNA, 558 bp, Artificial Sequence
  Nucleotide sequence encoding UL130_v1
tggagcacactgactgctaaccagaatccctccccaccctggagcaagctgacctactctaaacctcatgacgcagccacattctactgcccttttctgtatccaagtcctccaaggtctccaagtcagttcagcggatttcagcgagtgtctactgggcccgagtgtcgtaatgaaaccctgtacctgctgtataacagggagggacagaccctggtggaacggtccagcacatgggtgaagaaagtcatctggtatctgtctgggcggaatcagacaattctgcagaggatgccccggactgccagtaagccttcagacggaaacgtgcagatcagtgtcgaggatgctaaaattttcggggcacatatggtgcccaagcagacaaaactgctgagatttgtggtcaatgacggcactcgctaccagatgtgcgtgatgaagctggagagctgggcccacgtctttcgagattattcagtgtccttccaggtccgtctgacctttacagaagctaacaatcagacttacaccttctgtactcatcctaatctgatc

SEQ ID NO: 9 - protein, 22 aa, Artificial Sequence
  F2A
VKQTLNFDLLKLAGDVESNPGP

SEQ ID NO: 10 - DNA, 66 bp, Artificial Sequence
  F2A
gtgaagcagaccctgaactttgacctgctgaaactggcaggcgacgtggagagcaatccaggacca

SEQ ID NO: 11 - protein, 111 aa, Artificial Sequence
  UL131Av1
QCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 12 - DNA, 333 bp, Artificial Sequence
  Nucleotide sequence encoding UL131_v1
cagtgccagcgagagaccgcagaaaagaacgactactatcgggtgcctcactattgggacgcatgttctcgagccctgccagatcagacccgttacaaatatgtggagcagctggtcgacctgacactgaactaccactatgatgcctcccatggcctggacaatttcgatgtgctgaagagaatcaacgtgactgaagtctctctgctgattagtgatttcaggcggcagaacagacgcggcggaaccaataagcgcactacctttaacgctgcaggctccctggccccacatgctaggtcactggagttctccgtgcggctgtttgctaac

SEQ ID NO: 13 - protein, 7 aa, Artificial Sequence
  TEV site
ENLYFQG

SEQ ID NO: 14 - DNA, 21 bp, Artificial Sequence
  TEV site
gaaaatctgtactttcagggg

SEQ ID NO: 15 - protein, 4 aa, Artificial Sequence
  GS linker
SGSG

SEQ ID NO: 16 - DNA, 12 bp, Artificial Sequence
  GS linker
agcggttctggc

SEQ ID NO: 17 - protein, 20 aa, Artificial Sequence
  tandem Strep-tag_v1
WSHPQFEKGSGSWSHPQFEK

SEQ ID NO: 18 - DNA, 60 bp, Artificial Sequence
  tandem Strep-tag_v1
tggtcacacccacagtttgaaaaaggtagcggttcctggtcacacccacagtttgaaaaa

SEQ ID NO: 19 - protein, 19 aa, Artificial Sequence
  Mouse IgG signal peptide
MGWSCIILFLVATATGVHS

SEQ ID NO: 20 - DNA, 57 bp, Artificial Sequence
  Mouse IgG signal peptide nt seq
atgggttggtcctgtatcatcctgttcctggtcgctaccgctactggggtgcatagt

SEQ ID NO: 21 - protein, 688 aa, Artificial Sequence
  Amino acid sequence of gH_v1
GADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFPQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKTQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQSKCELTRNMHTTHSITAALNISLENCAFCQSALLEYDDTQGVINIMYMHDSDDVLFALDPYNEVVVSSPRTHYLMLLKNGTVLEVTDVVVD

SEQ ID NO: 22 - DNA, 2064 bp, Artificial Sequence
  Nucleotide Sequence encoding gH_v1
ggagcagacgcagcatcagaggccctggaccctcacgcttttcatctgctgctgaacacctacggccgaccaatccgtttcctgagggagaataccacacagtgcacatataactccagcctgagaaatagcactgtggtccgcgaaaacgccattagttttaatttctttcagtcatacaaccagtactatgtgttccacatgccaagatgtctgtttgctggacccctggcagagcagttcctgaaccaggtggatctgacagagactctggaaaggtaccagcagcggctgaatacctatgctctggtgtctaaggacctggcaagttatcgctcatttccccagcagctgaaagctcaggacagtctgggacagcagcctactaccgtcccacctccaatcgatctgtcaattcctcatgtgtggatgccccctcagacaactccacacgactggaaggggtcacataccacatccggtctgcacagaccacatttcaaccagacatgcatcctgtttgatggccacgacctgctgttctccaccgtgacaccctgtctgcatcagggattctacctgatggatgagctgagatatgtgaagattactctgaccgaagacttctttgtggtcaccgtgagcatcgacgatgacacaccaatgctgctgatttttggacacctgcctcgcgtgctgttcaaggccccataccagcgagataactttatcctgcgtcagactgagaaacacgaactgctggtcctggtgaagaaaacccagctgaatcgccattcatatctgaaagattccgactttctggatgccgctctggacttcaactacctggacctgtccgctctgctgcgaaatagcttccatcgttatgcagtcgatgtgctgaagagcggcaggtgccagatgctggacaggcggacagtggagatggcctttgcttacgcactggcactgttcgcagccgctcgacaggaggaagcaggaactgagatctctattcctagagctctggatcgccaggcagccctgctgcagatccaggagttcatgattacttgtctgtcccagaccccaccacgaactaccctgctgctgtacccaaccgccgtggacctggccaagagagctctgtggacacccgatcagatcactgacattaccagcctggtccgcctggtgtatatcctgtctaaacagaaccagcagcacctgatcccacagtgggccctgcgacagattgcagacttcgccctgcagctgcacaagacccatctggcttccttcctgagcgcttttgcacggcaggagctgtacctgatggggagcctggtccactctatgctggtgcatacaactgagagacgcgaaatctttattgtggagacaggtctgtgcagcctggccgaactgtctcacttcactcagctgctggctcatccacaccatgagtacctgtccgatctgtatactccctgttctagttcagggcgacgtgaccacagcctggaacggctgaccagactgtttcccgatgccaccgtcccagctacagtgcccgctgcactgtctatcctgagtaccatgcagccttctacactggagactttccccgatctgttttgcctgcctctgggcgagtcattctccgccctgaccgtcagtgaacatgtgtcatacgtggtcacaaaccagtatctgatcaaagggatttcctaccccgtgagcaccacagtggtcggccagtccctgatcattacacagactgacagccagtctaagtgtgagctgactaggaatatgcacactacccattcaatcaccgccgctctgaacatttccctggagaattgcgctttttgtcagagtgcactgctggaatacgatgacacccagggcgtgatcaacattatgtatatgcacgatagcgatgacgtgctgttcgctctggacccatacaacgaggtggtcgtgtccagcccacgaacccattatctgatgctgctgaagaatggtacagtcctggaagtgactgatgtcgtggtcgac

SEQ ID NO: 23 - protein, 19 aa, Artificial Sequence
  P2A
ATNFSLLKQAGDVEENPGP

SEQ ID NO: 24 - DNA, 57 bp, Artificial Sequence
  P2A nt seq
gcaacaaacttttcactgctgaaacaggccggggatgtggaggaaaatcctggtcca

SEQ ID NO: 25 - protein, 247 aa, Artificial Sequence
  Amino acid sequence of gL_v1
AVSVAPTAAEKVPAECPELTRRCLLGEVFQGDKYESWLRPLVNVTGRNGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 26 - DNA, 741 bp, Artificial Sequence
  Nucleotide sequence encoding gL_v1
gcagtctctgtggcaccaaccgcagcagagaaggtgcctgcagagtgcccagaactgacaaggcggtgtctgctgggcgaggtgttccagggagacaaatacgaatcctggctgagacccctggtcaacgtgacagggcgcaatggtcctctgtctcagctgatcagatatcgccccgtcactcctgaggctgcaaacagtgtgctgctggatgacgcatttctggataccctggccctgctgtacaacaatcctgaccagctgagggcactgctgaccctgctgtctagtgacacagctccaaggtggatgactgtgatgcggggctactctgaatgcggcgatggaagtcctgccgtctatacctgcgtggatgacctgtgtcgaggctacgacctgacaaggctgtcttatggacggagtatctttactgagcacgtcctgggattcgaactggtgcctccaagcctgttcaatgtggtcgtggccattcggaacgaggctacccgcacaaatcgagccgtccgtctgcccgtgtctactgccgctgcacctgagggcatcaccctgttttacggactgtataacgctgtgaaggagttctgtctgcgacaccagctggatccccctctgctgcgtcatctggacaagtactatgccggcctgccacccgagctgaaacagacaagagtcaacctgcccgcacacagccgctatggaccccaggccgtggatgctcgt

SEQ ID NO: 27 - protein, 22 aa, Artificial Sequence
  Amino acid sequence of P2A_v2
GSGATNFSLLKQAGDVEENPGP

SEQ ID NO: 28 - DNA, 66 bp, Artificial Sequence
  Nucleotide sequence encoding P2A_v2
ggaagcggagcaacaaacttttcactgctgaaacaggccggggatgtggaggaaaatcctggtcca

SEQ ID NO: 29 - protein, 26 aa, Artificial Sequence
  Amino acid sequence of P2A_v3
RKRRGSGATNFSLLKQAGDVEENPGP

SEQ ID NO: 30 - DNA, 78 bp, Artificial Sequence
  Nucleotide sequence encoding P2A_v3
cgcaaacgccgcggaagcggagcaacaaacttttcactgctgaaacaggccggggatgtggaggaaaatcctggtcca

SEQ ID NO: 31 - protein, 189 aa, Artificial Sequence
  Amino acid sequence encoding UL130_v2
SSWSTLTANQNPSPPWSKLTYSKPHDAATFYCPFLYPSPPRSPSQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 32 - DNA, 567 bp, Artificial Sequence
  Nucleotide sequence encoding UL130_v2
tcttcgtggagcacactgactgctaaccagaatccctccccaccctggagcaagctgacctactctaaacctcatgacgcagccacattctactgcccttttctgtatccaagtcctccaaggtctccaagtcagttcagcggatttcagcgagtgtctactgggcccgagtgtcgtaatgaaaccctgtacctgctgtataacagggagggacagaccctggtggaacggtccagcacatgggtgaagaaagtcatctggtatctgtctgggcggaatcagacaattctgcagaggatgccccggactgccagtaagccttcagacggaaacgtgcagatcagtgtcgaggatgctaaaattttcggggcacatatggtgcccaagcagacaaaactgctgagatttgtggtcaatgacggcactcgctaccagatgtgcgtgatgaagctggagagctgggcccacgtctttcgagattattcagtgtccttccaggtccgtctgacctttacagaagctaacaatcagacttacaccttctgtactcatcctaatctgatcgtt

SEQ ID NO: 33 - protein, 110 aa, Artificial Sequence
  Amino acid sequence of UL131_v2
CQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 34 - DNA, 330 bp, Artificial Sequence
  Nucleotide sequence encoding UL131_v2
tgccagcgagagaccgcagaaaagaacgactactatcgggtgcctcactattgggacgcatgttctcgagccctgccagatcagacccgttacaaatatgtggagcagctggtcgacctgacactgaactaccactatgatgcctcccatggcctggacaatttcgatgtgctgaagagaatcaacgtgactgaagtctctctgctgattagtgatttcaggcggcagaacagacgcggcggaaccaataagcgcactacctttaacgctgcaggctccctggccccacatgctaggtcactggagttctccgtgcggctgtttgctaac

SEQ ID NO: 35 - protein, 690 aa, Artificial Sequence
  gHv2
RYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFPQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKTQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQSKCELTRNMHTTHSITAALNISLENCAFCQSALLEYDDTQGVINIMYMHDSDDVLFALDPYNEVVVSSPRTHYLMLLKNGTVLEVTDVVVD

SEQ ID NO: 36 - DNA, 2070 bp, Artificial Sequence
  gHv2
cgctatggagcagacgcagcatcagaggccctggaccctcacgcttttcatctgctgctgaacacctacggccgaccaatccgtttcctgagggagaataccacacagtgcacatataactccagcctgagaaatagcactgtggtccgcgaaaacgccattagttttaatttctttcagtcatacaaccagtactatgtgttccacatgccaagatgtctgtttgctggacccctggcagagcagttcctgaaccaggtggatctgacagagactctggaaaggtaccagcagcggctgaatacctatgctctggtgtctaaggacctggcaagttatcgctcatttccccagcagctgaaagctcaggacagtctgggacagcagcctactaccgtcccacctccaatcgatctgtcaattcctcatgtgtggatgccccctcagacaactccacacgactggaaggggtcacataccacatccggtctgcacagaccacatttcaaccagacatgcatcctgtttgatggccacgacctgctgttctccaccgtgacaccctgtctgcatcagggattctacctgatggatgagctgagatatgtgaagattactctgaccgaagacttctttgtggtcaccgtgagcatcgacgatgacacaccaatgctgctgatttttggacacctgcctcgcgtgctgttcaaggccccataccagcgagataactttatcctgcgtcagactgagaaacacgaactgctggtcctggtgaagaaaacccagctgaatcgccattcatatctgaaagattccgactttctggatgccgctctggacttcaactacctggacctgtccgctctgctgcgaaatagcttccatcgttatgcagtcgatgtgctgaagagcggcaggtgccagatgctggacaggcggacagtggagatggcctttgcttacgcactggcactgttcgcagccgctcgacaggaggaagcaggaactgagatctctattcctagagctctggatcgccaggcagccctgctgcagatccaggagttcatgattacttgtctgtcccagaccccaccacgaactaccctgctgctgtacccaaccgccgtggacctggccaagagagctctgtggacacccgatcagatcactgacattaccagcctggtccgcctggtgtatatcctgtctaaacagaaccagcagcacctgatcccacagtgggccctgcgacagattgcagacttcgccctgcagctgcacaagacccatctggcttccttcctgagcgcttttgcacggcaggagctgtacctgatggggagcctggtccactctatgctggtgcatacaactgagagacgcgaaatctttattgtggagacaggtctgtgcagcctggccgaactgtctcacttcactcagctgctggctcatccacaccatgagtacctgtccgatctgtatactccctgttctagttcagggcgacgtgaccacagcctggaacggctgaccagactgtttcccgatgccaccgtcccagctacagtgcccgctgcactgtctatcctgagtaccatgcagccttctacactggagactttccccgatctgttttgcctgcctctgggcgagtcattctccgccctgaccgtcagtgaacatgtgtcatacgtggtcacaaaccagtatctgatcaaagggatttcctaccccgtgagcaccacagtggtcggccagtccctgatcattacacagactgacagccagtctaagtgtgagctgactaggaatatgcacactacccattcaatcaccgccgctctgaacatttccctggagaattgcgctttttgtcagagtgcactgctggaatacgatgacacccagggcgtgatcaacattatgtatatgcacgatagcgatgacgtgctgttcgctctggacccatacaacgaggtggtcgtgtccagcccacgaacccattatctgatgctgctgaagaatggtacagtcctggaagtgactgatgtcgtggtcgac

SEQ ID NO: 37 - protein, 248 aa, Artificial Sequence
  gLv2
VAVSVAPTAAEKVPAECPELTRRCLLGEVFQGDKYESWLRPLVNVTGRNGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 38 - DNA, 744 bp, Artificial Sequence
  gLv2 nt seq
gtggcagtctctgtggcaccaaccgcagcagagaaggtgcctgcagagtgcccagaactgacaaggcggtgtctgctgggcgaggtgttccagggagacaaatacgaatcctggctgagacccctggtcaacgtgacagggcgcaatggtcctctgtctcagctgatcagatatcgccccgtcactcctgaggctgcaaacagtgtgctgctggatgacgcatttctggataccctggccctgctgtacaacaatcctgaccagctgagggcactgctgaccctgctgtctagtgacacagctccaaggtggatgactgtgatgcggggctactctgaatgcggcgatggaagtcctgccgtctatacctgcgtggatgacctgtgtcgaggctacgacctgacaaggctgtcttatggacggagtatctttactgagcacgtcctgggattcgaactggtgcctccaagcctgttcaatgtggtcgtggccattcggaacgaggctacccgcacaaatcgagccgtccgtctgcccgtgtctactgccgctgcacctgagggcatcaccctgttttacggactgtataacgctgtgaaggagttctgtctgcgacaccagctggatccccctctgctgcgtcatctggacaagtactatgccggcctgccacccgagctgaaacagacaagagtcaacctgcccgcacacagccgctatggaccccaggccgtggatgctcgt

SEQ ID NO: 39 - protein, 31 aa, Artificial Sequence
  tandem strep-tag_v2
SAWSHPQFEKGGGSGGGSGGSSAWSHPQFEK

SEQ ID NO: 40 - DNA, 93 bp, Artificial Sequence
  tandem strep-tag_v2 nt seq
agcgcatggtcacacccacagtttgaaaaaggtggtggtagcggtggtggttccggtggtagcagcgcatggtcacacccacagtttgaaaaa

SEQ ID NO: 41 - protein, 6 aa, Artificial Sequence
  6xHis tag
HHHHHH

SEQ ID NO: 42 - DNA, 18 bp, Artificial Sequence
  6xHis tag nt seq
catcatcaccatcaccac

SEQ ID NO: 43 - protein, 523 aa, Artificial Sequence
  Amino acid sequence of pentamer_UL128-130-131A_v1
MSPKDLTPFLTALWLLLGHSRVPRVRAEECCEFINVNHPPERCYDFKMCNRFTVAYVLRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQWSTLTANQNPSPPWSKLTYSKPHDAATFYCPFLYPSPPRSPSQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIVKQTLNFDLLKLAGDVESNPGPQCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFANENLYFQGSGSGWSHPQFEKGSGSWSHPQFEK

SEQ ID NO: 44 - DNA, 1623 bp, Artificial Sequence
  Nucleotide sequence encoding pentamer_UL128-130-131A_v1
atgtctcctaaagacctgactccattcctgaccgccctgtggctgctgctggggcattcccgtgtccctcgtgtccgtgctgaagagtgctgtgagttcatcaacgtgaatcacccccctgaaaggtgctacgacttcaagatgtgtaataggttcaccgtggcttacgtcctgcgctgcccagatggcgaagtgtgttattcccccgagaagactgcagaaatcaggggcattgtcaccacaatgacacacagtctgactcggcaggtggtccataacaaactgacctcatgcaactacaatcctctgtatctggaagccgacggtcgaatccgttgtggcaaggtcaatgataaagctcagtacctgctgggagccgctggctcagtcccatacagatggattaacctggagtatgacaagatcacccgcattgtgggactggatcagtacctggaaagcgtgaagaaacacaagagactggacgtctgccgcgcaaaaatgggatatatgctgcaggaggggagagggtccctgctgacatgtggcgacgtggaggaaaaccccggcccttggagcacactgactgctaaccagaatccctccccaccctggagcaagctgacctactctaaacctcatgacgcagccacattctactgcccttttctgtatccaagtcctccaaggtctccaagtcagttcagcggatttcagcgagtgtctactgggcccgagtgtcgtaatgaaaccctgtacctgctgtataacagggagggacagaccctggtggaacggtccagcacatgggtgaagaaagtcatctggtatctgtctgggcggaatcagacaattctgcagaggatgccccggactgccagtaagccttcagacggaaacgtgcagatcagtgtcgaggatgctaaaattttcggggcacatatggtgcccaagcagacaaaactgctgagatttgtggtcaatgacggcactcgctaccagatgtgcgtgatgaagctggagagctgggcccacgtctttcgagattattcagtgtccttccaggtccgtctgacctttacagaagctaacaatcagacttacaccttctgtactcatcctaatctgatcgtgaagcagaccctgaactttgacctgctgaaactggcaggcgacgtggagagcaatccaggaccacagtgccagcgagagaccgcagaaaagaacgactactatcgggtgcctcactattgggacgcatgttctcgagccctgccagatcagacccgttacaaatatgtggagcagctggtcgacctgacactgaactaccactatgatgcctcccatggcctggacaatttcgatgtgctgaagagaatcaacgtgactgaagtctctctgctgattagtgatttcaggcggcagaacagacgcggcggaaccaataagcgcactacctttaacgctgcaggctccctggccccacatgctaggtcactggagttctccgtgcggctgtttgctaacgaaaatctgtactttcaggggagcggttctggctggtcacacccacagtttgaaaaaggtagcggttcctggtcacacccacagtttgaaaaa

SEQ ID NO: 45 - protein, 973 aa, Artificial Sequence
  Amino acid sequence of pentamer_gH-gL_v1
MGWSCIILFLVATATGVHSGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFPQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKTQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQSKCELTRNMHTTHSITAALNISLENCAFCQSALLEYDDTQGVINIMYMHDSDDVLFALDPYNEVVVSSPRTHYLMLLKNGTVLEVTDVVVDATNFSLLKQAGDVEENPGPAVSVAPTAAEKVPAECPELTRRCLLGEVFQGDKYESWLRPLVNVTGRNGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 46 - DNA, 2919 bp, Artificial Sequence
  Nucleotide sequence encoding pentamer_gH-gL_v1
atgggttggtcctgtatcatcctgttcctggtcgctaccgctactggggtgcatagtggagcagacgcagcatcagaggccctggaccctcacgcttttcatctgctgctgaacacctacggccgaccaatccgtttcctgagggagaataccacacagtgcacatataactccagcctgagaaatagcactgtggtccgcgaaaacgccattagttttaatttctttcagtcatacaaccagtactatgtgttccacatgccaagatgtctgtttgctggacccctggcagagcagttcctgaaccaggtggatctgacagagactctggaaaggtaccagcagcggctgaatacctatgctctggtgtctaaggacctggcaagttatcgctcatttccccagcagctgaaagctcaggacagtctgggacagcagcctactaccgtcccacctccaatcgatctgtcaattcctcatgtgtggatgccccctcagacaactccacacgactggaaggggtcacataccacatccggtctgcacagaccacatttcaaccagacatgcatcctgtttgatggccacgacctgctgttctccaccgtgacaccctgtctgcatcagggattctacctgatggatgagctgagatatgtgaagattactctgaccgaagacttctttgtggtcaccgtgagcatcgacgatgacacaccaatgctgctgatttttggacacctgcctcgcgtgctgttcaaggccccataccagcgagataactttatcctgcgtcagactgagaaacacgaactgctggtcctggtgaagaaaacccagctgaatcgccattcatatctgaaagattccgactttctggatgccgctctggacttcaactacctggacctgtccgctctgctgcgaaatagcttccatcgttatgcagtcgatgtgctgaagagcggcaggtgccagatgctggacaggcggacagtggagatggcctttgcttacgcactggcactgttcgcagccgctcgacaggaggaagcaggaactgagatctctattcctagagctctggatcgccaggcagccctgctgcagatccaggagttcatgattacttgtctgtcccagaccccaccacgaactaccctgctgctgtacccaaccgccgtggacctggccaagagagctctgtggacacccgatcagatcactgacattaccagcctggtccgcctggtgtatatcctgtctaaacagaaccagcagcacctgatcccacagtgggccctgcgacagattgcagacttcgccctgcagctgcacaagacccatctggcttccttcctgagcgcttttgcacggcaggagctgtacctgatggggagcctggtccactctatgctggtgcatacaactgagagacgcgaaatctttattgtggagacaggtctgtgcagcctggccgaactgtctcacttcactcagctgctggctcatccacaccatgagtacctgtccgatctgtatactccctgttctagttcagggcgacgtgaccacagcctggaacggctgaccagactgtttcccgatgccaccgtcccagctacagtgcccgctgcactgtctatcctgagtaccatgcagccttctacactggagactttccccgatctgttttgcctgcctctgggcgagtcattctccgccctgaccgtcagtgaacatgtgtcatacgtggtcacaaaccagtatctgatcaaagggatttcctaccccgtgagcaccacagtggtcggccagtccctgatcattacacagactgacagccagtctaagtgtgagctgactaggaatatgcacactacccattcaatcaccgccgctctgaacatttccctggagaattgcgctttttgtcagagtgcactgctggaatacgatgacacccagggcgtgatcaacattatgtatatgcacgatagcgatgacgtgctgttcgctctggacccatacaacgaggtggtcgtgtccagcccacgaacccattatctgatgctgctgaagaatggtacagtcctggaagtgactgatgtcgtggtcgacgcaacaaacttttcactgctgaaacaggccggggatgtggaggaaaatcctggtccagcagtctctgtggcaccaaccgcagcagagaaggtgcctgcagagtgcccagaactgacaaggcggtgtctgctgggcgaggtgttccagggagacaaatacgaatcctggctgagacccctggtcaacgtgacagggcgcaatggtcctctgtctcagctgatcagatatcgccccgtcactcctgaggctgcaaacagtgtgctgctggatgacgcatttctggataccctggccctgctgtacaacaatcctgaccagctgagggcactgctgaccctgctgtctagtgacacagctccaaggtggatgactgtgatgcggggctactctgaatgcggcgatggaagtcctgccgtctatacctgcgtggatgacctgtgtcgaggctacgacctgacaaggctgtcttatggacggagtatctttactgagcacgtcctgggattcgaactggtgcctccaagcctgttcaatgtggtcgtggccattcggaacgaggctacccgcacaaatcgagccgtccgtctgcccgtgtctactgccgctgcacctgagggcatcaccctgttttacggactgtataacgctgtgaaggagttctgtctgcgacaccagctggatccccctctgctgcgtcatctggacaagtactatgccggcctgccacccgagctgaaacagacaagagtcaacctgcccgcacacagccgctatggaccccaggccgtggatgctcgt

SEQ ID NO: 47 - protein, 521 aa, Artificial Sequence
  Amino acid sequence of Pentamer_UL128-130-131A_v3
MGWSCIILFLVATATGVHSPRVRAEECCEFINVNHPPERCYDFKMCNRFTVAYVLRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQRKRRGSGATNFSLLKQAGDVEENPGPSSWSTLTANQNPSPPWSKLTYSKPHDAATFYCPFLYPSPPRSPSQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIVRKRRGSGATNFSLLKQAGDVEENPGPCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 48 - DNA, 1563 bp, Artificial Sequence
  Nucleotide sequence encoding Pentamer_UL128-130-131A_v3
atgggttggtcctgtatcatcctgttcctggtcgctaccgctactggggtgcatagtcctcgtgtccgtgctgaagagtgctgtgagttcatcaacgtgaatcacccccctgaaaggtgctacgacttcaagatgtgtaataggttcaccgtggcttacgtcctgcgctgcccagatggcgaagtgtgttattcccccgagaagactgcagaaatcaggggcattgtcaccacaatgacacacagtctgactcggcaggtggtccataacaaactgacctcatgcaactacaatcctctgtatctggaagccgacggtcgaatccgttgtggcaaggtcaatgataaagctcagtacctgctgggagccgctggctcagtcccatacagatggattaacctggagtatgacaagatcacccgcattgtgggactggatcagtacctggaaagcgtgaagaaacacaagagactggacgtctgccgcgcaaaaatgggatatatgctgcagcgcaaacgccgcggaagcggagcaacaaacttttcactgctgaaacaggccggggatgtggaggaaaatcctggtccatcttcgtggagcacactgactgctaaccagaatccctccccaccctggagcaagctgacctactctaaacctcatgacgcagccacattctactgcccttttctgtatccaagtcctccaaggtctccaagtcagttcagcggatttcagcgagtgtctactgggcccgagtgtcgtaatgaaaccctgtacctgctgtataacagggagggacagaccctggtggaacggtccagcacatgggtgaagaaagtcatctggtatctgtctgggcggaatcagacaattctgcagaggatgccccggactgccagtaagccttcagacggaaacgtgcagatcagtgtcgaggatgctaaaattttcggggcacatatggtgcccaagcagacaaaactgctgagatttgtggtcaatgacggcactcgctaccagatgtgcgtgatgaagctggagagctgggcccacgtctttcgagattattcagtgtccttccaggtccgtctgacctttacagaagctaacaatcagacttacaccttctgtactcatcctaatctgatcgttcgcaaacgccgcggaagcggagcaacaaacttttcactgctgaaacaggccggggatgtggaggaaaatcctggtccatgccagcgagagaccgcagaaaagaacgactactatcgggtgcctcactattgggacgcatgttctcgagccctgccagatcagacccgttacaaatatgtggagcagctggtcgacctgacactgaactaccactatgatgcctcccatggcctggacaatttcgatgtgctgaagagaatcaacgtgactgaagtctctctgctgattagtgatttcaggcggcagaacagacgcggcggaaccaataagcgcactacctttaacgctgcaggctccctggccccacatgctaggtcactggagttctccgtgcggctgtttgctaac

SEQ ID NO: 49 - protein, 983 aa, Artificial Sequence
  Amino acid sequence of pentamer_gH-gL_v3
MGWSCIILFLVATATGVHSRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFPQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKTQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQSKCELTRNMHTTHSITAALNISLENCAFCQSALLEYDDTQGVINIMYMHDSDDVLFALDPYNEVVVSSPRTHYLMLLKNGTVLEVTDVVVDRKRRGSGATNFSLLKQAGDVEENPGPVAVSVAPTAAEKVPAECPELTRRCLLGEVFQGDKYESWLRPLVNVTGRNGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 50 - DNA, 2949 bp, Artificial Sequence
  Nucleotide sequence encoding pentamer_gH-gL_v3
atgggttggtcctgtatcatcctgttcctggtcgctaccgctactggggtgcatagtcgctatggagcagacgcagcatcagaggccctggaccctcacgcttttcatctgctgctgaacacctacggccgaccaatccgtttcctgagggagaataccacacagtgcacatataactccagcctgagaaatagcactgtggtccgcgaaaacgccattagttttaatttctttcagtcatacaaccagtactatgtgttccacatgccaagatgtctgtttgctggacccctggcagagcagttcctgaaccaggtggatctgacagagactctggaaaggtaccagcagcggctgaatacctatgctctggtgtctaaggacctggcaagttatcgctcatttccccagcagctgaaagctcaggacagtctgggacagcagcctactaccgtcccacctccaatcgatctgtcaattcctcatgtgtggatgccccctcagacaactccacacgactggaaggggtcacataccacatccggtctgcacagaccacatttcaaccagacatgcatcctgtttgatggccacgacctgctgttctccaccgtgacaccctgtctgcatcagggattctacctgatggatgagctgagatatgtgaagattactctgaccgaagacttctttgtggtcaccgtgagcatcgacgatgacacaccaatgctgctgatttttggacacctgcctcgcgtgctgttcaaggccccataccagcgagataactttatcctgcgtcagactgagaaacacgaactgctggtcctggtgaagaaaacccagctgaatcgccattcatatctgaaagattccgactttctggatgccgctctggacttcaactacctggacctgtccgctctgctgcgaaatagcttccatcgttatgcagtcgatgtgctgaagagcggcaggtgccagatgctggacaggcggacagtggagatggcctttgcttacgcactggcactgttcgcagccgctcgacaggaggaagcaggaactgagatctctattcctagagctctggatcgccaggcagccctgctgcagatccaggagttcatgattacttgtctgtcccagaccccaccacgaactaccctgctgctgtacccaaccgccgtggacctggccaagagagctctgtggacacccgatcagatcactgacattaccagcctggtccgcctggtgtatatcctgtctaaacagaaccagcagcacctgatcccacagtgggccctgcgacagattgcagacttcgccctgcagctgcacaagacccatctggcttccttcctgagcgcttttgcacggcaggagctgtacctgatggggagcctggtccactctatgctggtgcatacaactgagagacgcgaaatctttattgtggagacaggtctgtgcagcctggccgaactgtctcacttcactcagctgctggctcatccacaccatgagtacctgtccgatctgtatactccctgttctagttcagggcgacgtgaccacagcctggaacggctgaccagactgtttcccgatgccaccgtcccagctacagtgcccgctgcactgtctatcctgagtaccatgcagccttctacactggagactttccccgatctgttttgcctgcctctgggcgagtcattctccgccctgaccgtcagtgaacatgtgtcatacgtggtcacaaaccagtatctgatcaaagggatttcctaccccgtgagcaccacagtggtcggccagtccctgatcattacacagactgacagccagtctaagtgtgagctgactaggaatatgcacactacccattcaatcaccgccgctctgaacatttccctggagaattgcgctttttgtcagagtgcactgctggaatacgatgacacccagggcgtgatcaacattatgtatatgcacgatagcgatgacgtgctgttcgctctggacccatacaacgaggtggtcgtgtccagcccacgaacccattatctgatgctgctgaagaatggtacagtcctggaagtgactgatgtcgtggtcgaccgcaaacgccgcggaagcggagcaacaaacttttcactgctgaaacaggccggggatgtggaggaaaatcctggtccagtggcagtctctgtggcaccaaccgcagcagagaaggtgcctgcagagtgcccagaactgacaaggcggtgtctgctgggcgaggtgttccagggagacaaatacgaatcctggctgagacccctggtcaacgtgacagggcgcaatggtcctctgtctcagctgatcagatatcgccccgtcactcctgaggctgcaaacagtgtgctgctggatgacgcatttctggataccctggccctgctgtacaacaatcctgaccagctgagggcactgctgaccctgctgtctagtgacacagctccaaggtggatgactgtgatgcggggctactctgaatgcggcgatggaagtcctgccgtctatacctgcgtggatgacctgtgtcgaggctacgacctgacaaggctgtcttatggacggagtatctttactgagcacgtcctgggattcgaactggtgcctccaagcctgttcaatgtggtcgtggccattcggaacgaggctacccgcacaaatcgagccgtccgtctgcccgtgtctactgccgctgcacctgagggcatcaccctgttttacggactgtataacgctgtgaaggagttctgtctgcgacaccagctggatccccctctgctgcgtcatctggacaagtactatgccggcctgccacccgagctgaaacagacaagagtcaacctgcccgcacacagccgctatggaccccaggccgtggatgctcgt

SEQ ID NO: 51 - protein, 18 aa, Artificial Sequence
  peptide linker sequence
GSTSGSGXPGSGEGSTKG

SEQ ID NO: 52 - protein, 12 aa, Artificial Sequence
  Peptide linker sequence
GGGGSGGGGSGG

SEQ ID NO: 53 - protein, 20 aa, Artificial Sequence
  peptide linker sequence
GGGGSGGGGSGGGGSGGGGS

SEQ ID NO: 54 - protein, 15 aa, Artificial Sequence
  peptide linker sequence
GGGGSGGGGSGGGGS

SEQ ID NO: 55 - protein, 15 aa, Artificial Sequence
  peptide linker sequence
GVGGSGGGGSGGGGS

SEQ ID NO: 56 - protein, 8 aa, Artificial Sequence
  Amino acid sequence motif of ribosomal skipping site
DXEXNPGP

SEQ ID NO: 57 - protein, 8 aa, Artificial Sequence
  Amino acid sequence motif of ribosomal skipping site
DXEXNPGX

SEQ ID NO: 58 - protein, 1443 aa, Artificial Sequence
  Soluble Protein Complex with linkers
GADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFPQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKTQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQSKCELTRNMHTTHSITAALNISLENCAFCQSALLEYDDTQGVINIMYMHDSDDVLFALDPYNEVVVSSPRTHYLMLLKNGTVLEVTDVVVDGGGGSGGGGSGGGGSAVSVAPTAAEKVPAECPELTRRCLLGEVFQGDKYESWLRPLVNVTGRNGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDARGGGGSGGGGSGGGGSPRVRAEECCEFINVNHPPERCYDFKMCNRFTVAYVLRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQGGGGSGGGGSGGGGSWSTLTANQNPSPPWSKLTYSKPHDAATFYCPFLYPSPPRSPSQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIGGGGSGGGGSGGGGSQCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 59 - protein, 13 aa, Artificial Sequence
  His tag variant (for use at C-terminal end)
ENLYFQGHHHHHH